SEQUENCE LISTING (191 sequences)

SEQ ID NO: 1 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 432 aa, Artificial Sequence
  Description of Artificial Sequence Synthetic polypeptide
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAENLYFQGHHHHHH

SEQ ID NO: 3 - protein, 246 aa, Artificial Sequence
  Description of Artificial Sequence Synthetic polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDAHHHHHHHHHH

SEQ ID NO: 4 - protein, 125 aa, Mus musculus
QVTLKESGPGILQPSQTLSLTCSFDGFSLSTFGMGVGWIRQPSGRGLEWLAHIWWDDDKNYKPALKSRLTISKDTSKNQVFLKIANVDTADTATYYCTRMREDSSDYVYYIMDYWGQGTSVIVAS

SEQ ID NO: 5 - protein, 116 aa, Mus musculus
QMQLKQSGPGLVQPSQSLSITCTVSGFSLTSYGVHWVRQSPEKGLEWLGVIWSGGGTDYNAAFISRLSISKDNSKSQVFLKMSSLQTDDTAIYYCARGAYYSLDYWGQGTTLTVSS

SEQ ID NO: 6 - protein, 117 aa, Mus musculus
QVQLKQSGAELVRPGASVKLSCKASGYTFTDYYINWVKQRPGQGLEWIARIYPGNNYAYYDEKFKGKATLTAEKSSSTAYMQLSSLTSEDSAVYFCAKDDGYRFAYWGQGTLVTVSA

SEQ ID NO: 7 - protein, 119 aa, Mus musculus
EVQLQQSGPELVKPGASVKMSCKASGYSFTSYVMHWVKQKPGQGLEWIGYINPYNDGTKYNEKFKGKATLTSDKSSSTAFMDLSSLTSDDSAVYYCSRDDYSNPPFAYWGQGTLVTVSA

SEQ ID NO: 8 - protein, 113 aa, Mus musculus
EVQLQQSGAELVKPGASVKLSCTTSGFNIKDYYFHWVKQRTEQGLEWIGRIDPEDGETEYAPKFRDKATITADTSSNTAYLQLSSLTSEDTAIYYCARSLYYWGQGTTLTVSS

SEQ ID NO: 9 - protein, 122 aa, Mus musculus
QVTLKESGPGILQPSQTLSLTCSFSGFSLSTYGMGVGWIRQPSGKGLEWLANIWWNDDKNYNSALKSRLTISKDTSNNQLFLRISSVDTADTATYFCAQIPAYYSNSWFAYWGHGTLVTVSA

SEQ ID NO: 10 - protein, 118 aa, Mus musculus
EVQLQQSGPELVKPGDSVKMSCKASGYTFTDYYMDWVKQSHGKSLEWIGYIYTNNGDTNYNQKFKGKATLTVDKSSSTAYMELHSLTSEDSAVYYCASLYYSNFFDSWGQGTTLTVSS

SEQ ID NO: 11 - protein, 122 aa, Mus musculus
QVQLHQSGAELVKPGASVKLSCKASGYTFTEYSIHWVKQRSGQDLEWIGWFNPGTGSIKYNEKFKDKATLTADKSSSTVYMELSRLTSEDSAVYFCARHGGRWVIQWNFDVWGTGTTVTVSS

SEQ ID NO: 12 - protein, 122 aa, Mus musculus
QVQLQQPGAELVKPGTSVRLSCQASGYTFTSSWIHWVKQRTGQGLEWIGEISLTNGDINYNEKFTSKATLTVDKSSSTAYMQLSSLTSEDSAVYYCTIIPDGYSLYWYFDVWGAGTTVTVSS

SEQ ID NO: 13 - protein, 121 aa, Mus musculus
QVQLQQSGPELVKPGASVKISCKASGYAFSTSWMNWMKQRPGKGLEWIGRIYPGDGETNYNGKFKGKATLTADKSSSTAYMQLSSLTSDDSAVYFCARGANGDYLFYLDYWGQGTTLTVSS

SEQ ID NO: 14 - protein, 118 aa, Mus musculus
EVQLQQSGPELVKPGASVKMSCKASGYTFTSYVIHWVKQKPGQGLEWIGYINPYNDGTEYNEKFKGKATLTSDKSSSTAYMELSSLTSEDSAVYYCSRDDYNNRFAYWGPGTLVTVSA

SEQ ID NO: 15 - protein, 120 aa, Mus musculus
QVQLQQSGAELARPGASVKLSCKASGYSFTSYGISWVKQRTGQGLEWIGDIYPRSGNTDYNEKFKGKATLTADKSSSTANMQLSSLTSEDSAIYFCARSGDGYLYAMDYWGQGTSVTVSS

SEQ ID NO: 16 - protein, 116 aa, Mus musculus
QVQLKQSGPGLVQPSQSLSITCTVSGFSLSSYAVHWVRQSPGKGLEWLGVIWSGGTTDYNAAFISRLTISKDNSESQIFFKMNSLQPDDTAIYYCARGAYYSLDHWGQGTSLTVSS

SEQ ID NO: 17 - protein, 119 aa, Mus musculus
EVQLQQSGAELVSPGASVKLSCTASGFNIKDDYMHWVKQRPEQGLEWIGRIDPANGNTKYAPKFQDKATITADTSSNTAYLQLSSLTSEDTAVYYCFYSFYIPYTMDFWGQGTSVTVSS

SEQ ID NO: 18 - protein, 116 aa, Mus musculus
QVQVKQSGPGLVQPSQSLSITCTVSGFSSTSYAIHWVRQSPGKGLEWLGVIWSGGTTDYNAAFISRLSFSKDNSKSQLFFKMNSLQADDTAMYYCARGNYYGMDYWGQGTSVTVSS

SEQ ID NO: 19 - protein, 119 aa, Mus musculus
QVQLQQPGTELVKPGASVKLSCKASGYTFTSYWMHWVKQRPGQGLEWIGSVNPSNGGANYNEKFKNKATLTVDKSSSTAYMQLSSLTSEDSAVYYCARRNGFPYYFDYWGQGTTLTVSS

SEQ ID NO: 20 - protein, 119 aa, Mus musculus
QVQLQQPGAELVKPGASVKLSCKASGYAFTSSWINWVKRRPGQGLEWIGNVYPRSSSPNYNEKFKGKATLTVDTSSSTAYMQLSSLTSDDSAVYFCARGYGSYWYFDVWGAGTSVTVSS

SEQ ID NO: 21 - protein, 123 aa, Mus musculus
QVQLQQPGAELVKPGASVKLSCKASGYTFTSYWMQWVKQRPGQGLEWIGEIAPSDSYTNYNQKFKGKATLTVDTSSSTAYMQLSSLTSEDSAVYYCAGGDYYGYDVKTWFAYWGQGTLVTVSA

SEQ ID NO: 22 - protein, 118 aa, Mus musculus
QVQLQQPGAELVRPGSSVKLSCKASGYTFTNYWMLWVKQRPMQGLEWIGSIDPSDSETHYNQKFKDKATLTVDKSSSTAYMRLGSLTSEDSAVYYCARDSYGWYFDVWGTGTTVTVSS

SEQ ID NO: 23 - protein, 118 aa, Mus musculus
QVQLQQPGAELVKPGASVKLSCKASGYTFTDYWINWVKQRPGQGLEWIGNIYPGSSDTNYNEKFKRKATLTVDTSSSTAYMQLSSLTSDDSAVYYCARRPLFHAMDYWGQGTSVTVSS

SEQ ID NO: 24 - protein, 108 aa, Mus musculus
QIVLTQSPSIMSASPGEKVTISCSASSSVSYMYWYQQKPGSSPRPWIYRTSNLASGVPARFSGSGSGTSYSLTITSMEAEDAATYYCQLYHSYPPTWTFGGGTKLEIK

SEQ ID NO: 25 - protein, 111 aa, Mus musculus
DIVLTQSPASLAVSLGQRATISCRASESVDYDGDNYMNWYQQKPGQSPKLLIYATSNLESRIPARFSGSGSGTEFTLNIHPVEEEDAATYYCLQSYDVPYTFGSGTKLEMK

SEQ ID NO: 26 - protein, 107 aa, Mus musculus
DIQMTQSSSYLSVSLGGRVTITCKASDHINNWLAWYQQKPGNAPRLLISGATSLETGVPSRFSGSGSGKDYTLSITSLQTEDVATYYCQQYWSTPPTFGGGTKLEIK

SEQ ID NO: 27 - protein, 107 aa, Mus musculus
SIVMTQTPKFLLVSAGDRITITCKASQSVSNNVAWYQQKPGQSPTLLISYASNRFTGVPDRFTGSGYGTDFTFTISIVQAEDLAVYFCQQDYDSPYTFGGGTKLEIK

SEQ ID NO: 28 - protein, 113 aa, Mus musculus
DIVMTQSPSSLSVWAGEKVTMSCKSSQSLLNSGDQKNYLTWYQQKPGQSPKLLIYETSTRDYGVPDRFTGSGSGADFTLTISSVQAEDLAVYYCLNDLTYPLTFGAGTKLELK

SEQ ID NO: 29 - protein, 108 aa, Mus musculus
ENVLTQSPAIMAASPGEKVTMTCSASSSVSSGNFHWYQQKPGTSPKLWIYRTSNLASGVPARFSGSGSGTSYSLTISSMEAEDAATYYCQQWSGYPYTFGSGTKLKIK

SEQ ID NO: 30 - protein, 106 aa, Mus musculus
ENVLTQSPAIMSATLGEKVTVSCRARSNVKYMYWYQQKSGASPKLLIYYTSNLASGVPARFSGSGSGTSYSLTISSVEAEDAATYYCQQFTSSPFTFGSGTKLEIK

SEQ ID NO: 31 - protein, 113 aa, Mus musculus
DIVMSQSPSSLAVSVGEKVTMSCKSSQNLLYSSNQKNFLAWYQQRPGQSPKLLIYWASTRESGVPDRFTGSGTGTDFTLTISSVKAEDLAVYYCQQYYRYPFTFGSGTKLEIK

SEQ ID NO: 32 - protein, 106 aa, Mus musculus
QIVLSQSPPILSASPGARVTMTCRASSSVNYMHWYQQKPGSSPKPWIYATSNLASGVPVRFSGSGSGTSYSLTISRVEAEDAATYYCQQWSSNPPTFGGGTKLEIK

SEQ ID NO: 33 - protein, 107 aa, Mus musculus
DIQMTQTTSSLSASLGDRVTISCSASQDISDFLNWYQQKPDGTVKLLIYYTSSLHSGVPSRFSGSGSGTDYSLTINNLEPEDIATYYCQQYSKLPYTFGGGTKLEIR

SEQ ID NO: 34 - protein, 107 aa, Mus musculus
DIQMTQSPASLSASVGETVTITCRPSENIYSYLAWYQQKQGKSPQLLVYNAKTLVEGVPSRFSGSGSGTQFSLKINSVQPEDCGSYYCLHHFGTPWTFGGGTILEIK

SEQ ID NO: 35 - protein, 107 aa, Mus musculus
DIQMTQTTSSLSASLGDRVTISCRASQDISNYLNWYQQKPDGTVKLLIYYTSRLHSGVPSRFSGSGSGTDYSLTISNLEPEDIAPYYCQQYSKLPYTFGSGTKLEIK

SEQ ID NO: 36 - protein, 111 aa, Mus musculus
DVVVTQTPLSLPVSFGDQVSISCRSTQSLANSYGNTYLSWYLHKPGQSPQLLIYGISNRFSGVPDRFSGSGSGTDFTLKISTLKPEDLGMYYYLQGTHQYTFGGGTKLEIK

SEQ ID NO: 37 - protein, 106 aa, Mus musculus
ENVLTQSPAIMSATLGEKVTMSCRASSNVKYMYWYQQKSGASPKLWIYYTSNLTSGVPARFSGSGSGTSYSLTISSVEAEDAATYYCQQFTSSPYTFGSGTKLEIK

SEQ ID NO: 38 - protein, 111 aa, Mus musculus
DIVVTQSPASLAVSLGQRATISCRASESIDYDGDSYVNWYQQKPGQPPNLLIYAASNLESGIPARFSGGGSGTDFTLTIHPVEEEDAATYYCLQSYETPWTFGGGTKLEIK

SEQ ID NO: 39 - protein, 107 aa, Mus musculus
DIVMTQSQKFVSTSVGDRVSVTCKASQNVGTNVVWYQQKPGQSPKPLIYSASYRYSGVPDRFTGSGSGTDFTLTISNVQSEDLADYFCHQYNSSPLTFGVGTKLELK

SEQ ID NO: 40 - protein, 107 aa, Mus musculus
DIQMTQSSSSFSVSLGDRVTITCKASEDIYNRLAWYQQKPGNAPRLLISGATTLETGVPSRFSGSGSGKDYALSITSLQTEDVSTYYCQEYWSLSPTFGGGTKLEIK

SEQ ID NO: 41 - protein, 107 aa, Mus musculus
DIQMTQTTSSLSASLGDRVTISCSASQDISNYLNWYQQKPDGTVKLLIYYTSSLHSGVPSRFSGSGSGTGYSLTISNLEPEDIATYYCQQYSKLPYTFGGGTKLEIK

SEQ ID NO: 42 - protein, 107 aa, Mus musculus
DIVMTQSQKFMSTSVGDRVSVTCKASQNVGSNVAWYQQKSGQSPKALIYSASYRYSGVPDRFTGSGYGTDFTLTISNVQSEDLAEYFCQQYNNYPLTFGAGTKLELK

SEQ ID NO: 43 - protein, 111 aa, Mus musculus
DIVLTQSPASLAVTLGQRATISCKASQSINYNGDNYMHWFQQKPGQPTKLLIYTASNLESGVPDRFSGSGSGTDFTLNIHPVEEEDAATYYCQQSKEFPWTFGGGTKLEIK

SEQ ID NO: 44 - protein, 10 aa, Mus musculus
GFSLSTFGMG

SEQ ID NO: 45 - protein, 6 aa, Mus musculus
IWWDDD

SEQ ID NO: 46 - protein, 17 aa, Mus musculus
TRMREDSSDYVYYIMDY

SEQ ID NO: 47 - protein, 8 aa, Mus musculus
GFSLTSYG

SEQ ID NO: 48 - protein, 6 aa, Mus musculus
IWSGGG

SEQ ID NO: 49 - protein, 10 aa, Mus musculus
ARGAYYSLDY

SEQ ID NO: 50 - protein, 8 aa, Mus musculus
GYTFTDYY

SEQ ID NO: 51 - protein, 7 aa, Mus musculus
IYPGNNY

SEQ ID NO: 52 - protein, 10 aa, Mus musculus
AKDDGYRFAY

SEQ ID NO: 53 - protein, 8 aa, Mus musculus
GYSFTSYV

SEQ ID NO: 54 - protein, 7 aa, Mus musculus
INPYNDG

SEQ ID NO: 55 - protein, 12 aa, Mus musculus
SRDDYSNPPFAY

SEQ ID NO: 56 - protein, 8 aa, Mus musculus
GFNIKDYY

SEQ ID NO: 57 - protein, 7 aa, Mus musculus
IDPEDGE

SEQ ID NO: 58 - protein, 6 aa, Mus musculus
ARSLYY

SEQ ID NO: 59 - protein, 10 aa, Mus musculus
GFSLSTYGMG

SEQ ID NO: 60 - protein, 6 aa, Mus musculus
IWWNDD

SEQ ID NO: 61 - protein, 14 aa, Mus musculus
AQIPAYYSNSWFAY

SEQ ID NO: 62 - protein, 7 aa, Mus musculus
IYTNNGD

SEQ ID NO: 63 - protein, 11 aa, Mus musculus
ASLYYSNFFDS

SEQ ID NO: 64 - protein, 8 aa, Mus musculus
GYTFTEYS

SEQ ID NO: 65 - protein, 7 aa, Mus musculus
FNPGTGS

SEQ ID NO: 66 - protein, 15 aa, Mus musculus
ARHGGRWVIQWNFDV

SEQ ID NO: 67 - protein, 8 aa, Mus musculus
GYTFTSSW

SEQ ID NO: 68 - protein, 7 aa, Mus musculus
ISLTNGD

SEQ ID NO: 69 - protein, 15 aa, Mus musculus
TIIPDGYSLYWYFDV

SEQ ID NO: 70 - protein, 8 aa, Mus musculus
GYAFSTSW

SEQ ID NO: 71 - protein, 7 aa, Mus musculus
IYPGDGE

SEQ ID NO: 72 - protein, 14 aa, Mus musculus
ARGANGDYLFYLDY

SEQ ID NO: 73 - protein, 8 aa, Mus musculus
GYTFTSYV

SEQ ID NO: 74 - protein, 11 aa, Mus musculus
SRDDYNNRFAY

SEQ ID NO: 75 - protein, 8 aa, Mus musculus
GYSFTSYG

SEQ ID NO: 76 - protein, 7 aa, Mus musculus
IYPRSGN

SEQ ID NO: 77 - protein, 13 aa, Mus musculus
ARSGDGYLYAMDY

SEQ ID NO: 78 - protein, 8 aa, Mus musculus
GFSLSSYA

SEQ ID NO: 79 - protein, 6 aa, Mus musculus
IWSGGT

SEQ ID NO: 80 - protein, 10 aa, Mus musculus
ARGAYYSLDH

SEQ ID NO: 81 - protein, 8 aa, Mus musculus
GFNIKDDY

SEQ ID NO: 82 - protein, 7 aa, Mus musculus
IDPANGN

SEQ ID NO: 83 - protein, 12 aa, Mus musculus
FYSFYIPYTMDF

SEQ ID NO: 84 - protein, 8 aa, Mus musculus
GFSSTSYA

SEQ ID NO: 85 - protein, 10 aa, Mus musculus
ARGNYYGMDY

SEQ ID NO: 86 - protein, 8 aa, Mus musculus
GYTFTSYW

SEQ ID NO: 87 - protein, 7 aa, Mus musculus
VNPSNGG

SEQ ID NO: 88 - protein, 12 aa, Mus musculus
ARRNGFPYYFDY

SEQ ID NO: 89 - protein, 8 aa, Mus musculus
GYAFTSSW

SEQ ID NO: 90 - protein, 7 aa, Mus musculus
VYPRSSS

SEQ ID NO: 91 - protein, 12 aa, Mus musculus
ARGYGSYWYFDV

SEQ ID NO: 92 - protein, 7 aa, Mus musculus
IAPSDSY

SEQ ID NO: 93 - protein, 16 aa, Mus musculus
AGGDYYGYDVKTWFAY

SEQ ID NO: 94 - protein, 8 aa, Mus musculus
GYTFTNYW

SEQ ID NO: 95 - protein, 7 aa, Mus musculus
IDPSDSE

SEQ ID NO: 96 - protein, 11 aa, Mus musculus
ARDSYGWYFDV

SEQ ID NO: 97 - protein, 8 aa, Mus musculus
GYTFTDYW

SEQ ID NO: 98 - protein, 7 aa, Mus musculus
IYPGSSD

SEQ ID NO: 99 - protein, 11 aa, Mus musculus
ARRPLFHAMDY

SEQ ID NO: 100 - protein, 5 aa, Mus musculus
SSVSY

SEQ ID NO: 101 - protein, 3 aa, Mus musculus
RTS

SEQ ID NO: 102 - protein, 11 aa, Mus musculus
QLYHSYPPTWT

SEQ ID NO: 103 - protein, 10 aa, Mus musculus
ESVDYDGDNY

SEQ ID NO: 104 - protein, 3 aa, Mus musculus
ATS

SEQ ID NO: 105 - protein, 9 aa, Mus musculus
LQSYDVPYT

SEQ ID NO: 106 - protein, 6 aa, Mus musculus
DHINNW

SEQ ID NO: 107 - protein, 3 aa, Mus musculus
GAT

SEQ ID NO: 108 - protein, 9 aa, Mus musculus
QQYWSTPPT

SEQ ID NO: 109 - protein, 6 aa, Mus musculus
QSVSNN

SEQ ID NO: 110 - protein, 3 aa, Mus musculus
YAS

SEQ ID NO: 111 - protein, 9 aa, Mus musculus
QQDYDSPYT

SEQ ID NO: 112 - protein, 12 aa, Mus musculus
QSLLNSGDQKNY

SEQ ID NO: 113 - protein, 3 aa, Mus musculus
ETS

SEQ ID NO: 114 - protein, 9 aa, Mus musculus
LNDLTYPLT

SEQ ID NO: 115 - protein, 7 aa, Mus musculus
SSVSSGN

SEQ ID NO: 116 - protein, 9 aa, Mus musculus
QQWSGYPYT

SEQ ID NO: 117 - protein, 5 aa, Mus musculus
SNVKY

SEQ ID NO: 118 - protein, 3 aa, Mus musculus
YTS

SEQ ID NO: 119 - protein, 9 aa, Mus musculus
QQFTSSPFT

SEQ ID NO: 120 - protein, 12 aa, Mus musculus
QNLLYSSNQKNF

SEQ ID NO: 121 - protein, 3 aa, Mus musculus
WAS

SEQ ID NO: 122 - protein, 9 aa, Mus musculus
QQYYRYPFT

SEQ ID NO: 123 - protein, 5 aa, Mus musculus
SSVNY

SEQ ID NO: 124 - protein, 9 aa, Mus musculus
QQWSSNPPT

SEQ ID NO: 125 - protein, 6 aa, Mus musculus
QDISDF

SEQ ID NO: 126 - protein, 9 aa, Mus musculus
QQYSKLPYT

SEQ ID NO: 127 - protein, 6 aa, Mus musculus
ENIYSY

SEQ ID NO: 128 - protein, 3 aa, Mus musculus
NAK

SEQ ID NO: 129 - protein, 9 aa, Mus musculus
LHHFGTPWT

SEQ ID NO: 130 - protein, 6 aa, Mus musculus
QDISNY

SEQ ID NO: 131 - protein, 11 aa, Mus musculus
QSLANSYGNTY

SEQ ID NO: 132 - protein, 3 aa, Mus musculus
GIS

SEQ ID NO: 133 - protein, 8 aa, Mus musculus
LQGTHQYT

SEQ ID NO: 134 - protein, 9 aa, Mus musculus
QQFTSSPYT

SEQ ID NO: 135 - protein, 10 aa, Mus musculus
ESIDYDGDSY

SEQ ID NO: 136 - protein, 3 aa, Mus musculus
AAS

SEQ ID NO: 137 - protein, 9 aa, Mus musculus
LQSYETPWT

SEQ ID NO: 138 - protein, 6 aa, Mus musculus
QNVGTN

SEQ ID NO: 139 - protein, 3 aa, Mus musculus
SAS

SEQ ID NO: 140 - protein, 9 aa, Mus musculus
HQYNSSPLT

SEQ ID NO: 141 - protein, 6 aa, Mus musculus
EDIYNR

SEQ ID NO: 142 - protein, 9 aa, Mus musculus
QEYWSLSPT

SEQ ID NO: 143 - protein, 6 aa, Mus musculus
QNVGSN

SEQ ID NO: 144 - protein, 9 aa, Mus musculus
QQYNNYPLT

SEQ ID NO: 145 - protein, 10 aa, Mus musculus
QSINYNGDNY

SEQ ID NO: 146 - protein, 3 aa, Mus musculus
TAS

SEQ ID NO: 147 - protein, 9 aa, Mus musculus
QQSKEFPWT

SEQ ID NO: 148 - DNA, 375 bp, Mus musculus
caggttactctgaaagagtctggccctgggatattgcagccctcccagaccctcagtctgacttgttctttcgatgggttttcactgagcacttttggtatgggtgtcggctggattcgtcagccttcagggaggggtctggagtggctggcacacatttggtgggatgatgataagaactataaaccagccctgaagagccggctcacaatctccaaggatacctccaaaaaccaggtattcctcaagatcgccaatgtggacactgcagatactgccacatactactgtactcgaatgagggaggacagctcggactacgtttattatattatggactattggggtcaaggaacctcagtcatcgtcgcctca

SEQ ID NO: 149 - DNA, 348 bp, Mus musculus
cagatgcagctgaagcagtcaggacctggcctagtgcagccctcacagagcctgtccatcacctgcacagtctctggtttctcattaactagctatggtgttcactgggttcgccagtctccagaaaagggtctggagtggctgggagtgatatggagtggtggaggtacagactataatgcagctttcatatccagactgagcatcagcaaggacaattccaagagccaagttttccttaaaatgagcagtctgcaaactgatgacacagccatatattactgtgccagaggggcctactatagtcttgactactggggccaaggcaccactctcacagtctcctca

SEQ ID NO: 150 - DNA, 351 bp, Mus musculus
caggtccagctgaagcagtctggggctgagctggtgaggcctggggcttcagtgaagctgtcctgcaaggcttctggctacactttcactgactactatataaactgggtgaagcagaggcctggacagggacttgagtggattgcaaggatttatcctggaaataattatgcttactacgatgagaagttcaagggcaaggccacactgactgcagaaaaatcctccagcactgcctacatgcagctcagcagcctgacatctgaggactctgctgtctatttctgtgcaaaggatgatggttaccggtttgcttactggggccaagggactctggtcactgtctctgca

SEQ ID NO: 151 - DNA, 357 bp, Mus musculus
gaggtccagctgcagcagtctggacctgagctggtaaagccaggggcttcagtgaagatgtcctgcaaggcttctggatactcattcactagttatgttatgcactgggtgaagcagaagcctgggcagggccttgaatggattggatatattaatccttacaatgatggtactaagtacaatgagaagttcaaaggcaaggccacactgacttcagacaaatcctccagcacagccttcatggacctcagcagcctgacctctgatgactctgcggtctattactgttcaagagacgactatagtaaccccccttttgcttactggggccaagggactttggtcactgtctctgca

SEQ ID NO: 152 - DNA, 339 bp, Mus musculus
gaggttcagctgcagcagtctggggcagagcttgtgaagccaggggcctcagtcaagttgtcctgcacaacttctggcttcaacattaaagactactattttcactgggtgaaacagaggactgagcagggcctggagtggattggaaggattgatcctgaggatggtgaaactgaatatgccccgaaattccgggacaaggccactataacagcagacacatcctccaacacagcctacctgcagctcagcagcctgacatctgaggacactgccatctattactgtgctagatccctctactactggggccaaggcaccactctcacagtctcctca

SEQ ID NO: 153 - DNA, 366 bp, Mus musculus
caggtcactctgaaagagtctggccctgggatattgcagccctcccagaccctcagtctgacttgttctttctctgggttttcactgagcacttatggtatgggtgtaggttggattcgtcagccttcagggaagggtctggagtggctggccaacatttggtggaatgatgataagaactataattcagccctgaagagccggctcacaatctccaaggatacctccaacaaccaactattcctcaggatctccagtgtggacactgcagatactgccacatacttctgtgctcaaatacctgcctactatagtaactcctggtttgcttactggggccacgggactctggtcactgtctctgca

SEQ ID NO: 154 - DNA, 354 bp, Mus musculus
gaagtccagctacaacagtctggacctgagctggtgaagcctggggattcagtgaagatgtcctgcaaggcttctggctacacattcactgactactacatggactgggtgaagcagagccatggaaagagccttgagtggattggatatatttatactaacaatggtgatactaactacaatcagaagttcaagggcaaggccacattgactgtagacaagtcctccagtacagcctacatggagctccacagcctgacatctgaggactctgcagtctattactgtgcaagtctctactatagtaacttttttgactcctggggccaaggcaccactctcacagtctcctca

SEQ ID NO: 155 - DNA, 366 bp, Mus musculus
caggtccagctgcaccagtctggagctgagctggtgaaacccggggcatcagtgaagctgtcctgcaaggcttctggctacaccttcactgagtatagtattcactgggtaaagcagaggtctggacaggatcttgagtggattgggtggtttaaccctggaacaggtagtataaagtacaatgagaaattcaaggacaaggccacattgactgcggacaaatcctccagcacagtctatatggagcttagtagattgacatctgaagactctgcggtctatttctgtgcaagacacggaggcagatgggtaatacagtggaacttcgatgtctggggcacagggaccacggtcaccgtctcctca

SEQ ID NO: 156 - DNA, 366 bp, Mus musculus
caggtccaattgcagcagcctggggctgagcttgtgaagcctgggacttcagtgaggttgtcctgccaggcttctggctacaccttcaccagctcctggatacactgggtgaagcagaggactggacaaggccttgagtggattggggagattagtcttaccaatggtgatattaactacaatgagaagttcacgagcaaggccacactgactgttgacaaatcctccagcaccgcctacatgcaacttagcagcctgacatctgaggactctgcggtctattactgtacaataatccctgatggttactccctctactggtacttcgatgtctggggcgcagggaccacggtcaccgtctcctca

SEQ ID NO: 157 - DNA, 363 bp, Mus musculus
caggttcaactgcagcagtctggacctgagctggtgaagcctggggcctcagtgaagatttcctgcaaggcttctggctacgcattcagtacctcctggatgaactggatgaagcagaggcctggaaagggtcttgagtggattggacggatttatcctggagatggagaaactaattataatgggaaattcaagggcaaggccacactgactgcagacaaatcctccagcacagcctacatgcaactcagcagcctgacatctgacgactctgcggtctacttctgtgcaagaggggccaatggtgactacttgttctaccttgactactggggccaaggcaccactctcacagtctcctca

SEQ ID NO: 158 - DNA, 354 bp, Mus musculus
gaggtccagctgcagcagtctggacctgagctggtaaagcctggggcttcagtgaagatgtcctgcaaggcttctggatacacattcactagctatgttatacactgggtgaagcagaagcctgggcagggccttgagtggattggatatattaatccttacaatgatggtactgagtacaatgagaagttcaagggcaaggccacactgacttcagacaaatcctccagtacagcctacatggagctcagcagcctgacctctgaggactctgcggtctattactgttcaagagatgactataataaccggtttgcttactggggcccagggactctggtcactgtctctgca

SEQ ID NO: 159 - DNA, 360 bp, Mus musculus
caggttcagctgcagcagtctggagctgagctggcgaggcctggggcttcagtgaagctgtcctgcaaggcttctggctacagtttcacaagctatggtataagttgggtgaagcagagaactggacagggccttgagtggattggagacatttatcctagaagtggtaatactgactacaatgagaagttcaagggcaaggccacactgactgcagacaaatcctccagcacagccaacatgcagctcagcagcctgacatctgaggactctgcaatctatttctgtgcaagatcgggagatggttacctctatgctatggactactggggtcaaggaacctcagtcaccgtctcctca

SEQ ID NO: 160 - DNA, 348 bp, Mus musculus
caggtgcaactgaagcagtcaggacctggcctagtgcagccctcacagagcctgtccatcacctgcacagtctctggtttctcattaagtagctatgctgtacactgggttcgccagtctccaggaaagggtctggagtggctgggagtgatatggagtggtggaaccacagactataatgcagctttcatatccagactgaccatcagcaaggacaattccgagagccaaattttctttaaaatgaacagtcttcaacctgatgacacagccatatattattgtgccagaggggcctactatagtcttgaccactggggccaaggcacctctctcacagtctcctca

SEQ ID NO: 161 - DNA, 357 bp, Mus musculus
gaggttcagctgcagcagtctggggctgagcttgtgagtccaggggcctcagtcaagttgtcctgcacagcttctggctttaacattaaagacgactatatgcactgggtgaagcagaggcctgaacagggcctggagtggattggaaggattgatcctgcgaatggtaatactaaatatgccccgaagttccaggacaaggccactataactgcagacacatcctccaacacagcctacctgcagctcagcagcctgacatctgaggacactgccgtctattactgtttctatagtttctatattccttatactatggacttctggggtcaaggaacctcagtcaccgtctcctca

SEQ ID NO: 162 - DNA, 348 bp, Mus musculus
caggtgcaggtgaagcagtcaggacctggcctggtgcagccctcacagagcctgtccatcacctgcacagtctctggtttctcatcaactagttatgctatacactgggttcgccagtctccaggaaagggtctggagtggctgggagtgatatggagtggtggaaccacagactataatgcagctttcatatccagactgagcttcagcaaggacaattccaagagccaacttttctttaaaatgaacagtctgcaagctgatgacacagccatgtattactgtgccagaggtaattactatggtatggactactggggtcaaggaacctcggtcaccgtctcctca

SEQ ID NO: 163 - DNA, 357 bp, Mus musculus
caggtccaactgcagcagcctgggactgaactggtgaagcctggggcttcagtgaagctgtcctgcaaggcttctggctacaccttcaccagctactggatgcactgggtgaagcagaggcctggacaaggccttgagtggattggaagtgttaatcctagcaatggtggtgctaactacaatgagaagttcaagaacaaggccacactgactgtagacaaatcctccagcacagcctacatgcaactcagcagcctgacatctgaggactctgcggtctattattgtgcaagaaggaatggtttcccgtactactttgactactggggccaaggcaccactctcacagtctcctca

SEQ ID NO: 164 - DNA, 357 bp, Mus musculus
caggtccaactgcagcagcctggtgctgagcttgtgaagcctggggcctcagtgaagctgtcctgcaaggcttctggctacgctttcaccagctcctggataaactgggtgaagcggaggcctggacaaggccttgagtggattggaaatgtttatcctcgtagtagtagtcctaactacaatgagaagttcaagggcaaggccacactgactgttgacacatcctccagcacagcctacatgcagctcagcagcctgacatctgacgactctgcggtctatttttgtgcaagaggatatggtagttactggtacttcgatgtctggggcgcagggacctcggtcaccgtctcctca

SEQ ID NO: 165 - DNA, 369 bp, Mus musculus
caggtccaactgcagcagcctggggctgagcttgtgaagcctggggcttcagtgaagctgtcctgcaaggcttctggctacaccttcaccagctactggatgcagtgggtaaaacagaggcctggacagggccttgagtggatcggagagattgctccttctgatagctatactaactacaatcaaaagttcaagggcaaggccacattgactgtagacacatcctccagcacagcctacatgcagctcagcagcctgacatctgaggactctgcggtctattactgtgcaggaggggactactatggttacgacgtaaagacttggtttgcttactggggccaagggactctggtcactgtctctgca

SEQ ID NO: 166 - DNA, 354 bp, Mus musculus
caggtccaactgcagcagcctggggctgagctggtgaggcctgggtcttcagtgaaactgtcctgcaaggcttctggctacaccttcaccaactactggatgctttgggtgaagcagaggcctatgcaaggccttgaatggattggtagcattgacccttctgatagtgaaactcactacaatcaaaagttcaaggacaaggccacattgactgtagacaaatcctccagcacagcctacatgcggctcggcagcctgacatctgaggactctgcggtctattactgtgcaagagattcctacggttggtacttcgatgtctggggcacagggaccacggtcaccgtctcctca

SEQ ID NO: 167 - DNA, 354 bp, Mus musculus
caggtccaattgcagcagcctggtgctgagcttgtgaagcctggggcctcagtgaagctgtcctgcaaggcttctggctacactttcaccgactactggataaactgggtgaagcagaggcctggacaaggccttgagtggattggaaatatttatcctggtagtagtgatactaactacaatgagaagttcaagcgcaaggccacactgactgtagacacatcctccagcacagcctacatgcagctcagcagcctgacatctgacgactctgcggtctattattgtgcaagacggcctctgttccatgctatggactactggggtcaaggaacctcagtcaccgtctcctca

SEQ ID NO: 168 - DNA, 324 bp, Mus musculus
caaattgttctcacccagtctccatcaatcatgtctgcatctccaggggagaaggtcaccatatcctgcagtgccagctcaagtgtaagttacatgtactggtaccagcagaagccaggatcctcccccagaccctggatttatcgcacatccaacctggcttctggagtccctgctcgcttcagtggcagtgggtctgggacctcttactctctcacaatcaccagcatggaggctgaagatgctgccacttattactgccagctgtatcatagttacccacccacgtggacgttcggtggaggcaccaagctggaaatcaaa

SEQ ID NO: 169 - DNA, 333 bp, Mus musculus
gacattgtgctgacccaatctccagcttcattggctgtgtctctagggcagagggccaccatctcctgcagagccagcgaaagtgttgattatgatggcgataattatatgaactggtaccaacagaaaccaggacagtcaccgaaactcctcatctacgctacatccaatctagaatctaggatccctgccaggtttagtggcagtgggtctgggacagaattcaccctcaacattcatcctgtggaggaggaagatgctgcaacctattactgtctgcaaagttatgacgttccgtatacgttcggatcggggaccaagctggaaatgaaa

SEQ ID NO: 170 - DNA, 321 bp, Mus musculus
gacatccagatgacacaatcttcatcctacttgtctgtatctctaggaggcagagtcaccattacttgcaaggcaagtgaccacattaataattggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgctacttattactgtcaacagtattggagtactcctccgacgttcggtggaggcaccaagctggaaatcaaa

SEQ ID NO: 171 - DNA, 321 bp, Mus musculus
agtattgtgatgacccagactcccaaattcctgcttgtatcagcaggagacaggattaccataacctgcaaggccagtcagagtgtgagtaataatgtagcttggtaccaacagaagccagggcagtctcctacactgctgatatcctatgcatccaatcgcttcactggagtccctgatcgcttcactggcagtggatatgggacggatttcactttcaccatcagcattgtgcaggctgaagacctggcagtttatttctgtcagcaggattatgactctccgtacacgttcggaggggggaccaagctggaaataaaa

SEQ ID NO: 172 - DNA, 339 bp, Mus musculus
gacattgtgatgacacagtctccatcctccctgagtgtgtgggcaggagagaaggtcactatgagctgcaagtccagtcagagtctgttaaacagtggagatcagaagaactacttgacctggtaccagcagaaaccaggacagtctcctaaactgttgatctacgagacatccactagggattatggggtccctgatcgcttcacaggcagtggatctggagccgatttcactcttaccatcagcagtgtgcaggctgaagacctggcagtttattactgtctgaatgatcttacttatccgctcacattcggtgctgggaccaagctggaactgaaa

SEQ ID NO: 173 - DNA, 324 bp, Mus musculus
gaaaatgtgctgacccagtctccagcaatcatggctgcatctccaggggagaaggtcaccatgacctgcagtgccagctcaagtgtaagttctggtaactttcactggtaccagcagaagccaggcacttctcccaaactctggatttataggacatccaacctggcttctggagtccccgctcgcttcagtggcagtgggtctgggacctcttactctcttacaatcagcagcatggaggccgaggatgctgccacttattactgccagcagtggagtggttacccgtatacgttcggatcggggaccaaactgaaaataaaa

SEQ ID NO: 174 - DNA, 318 bp, Mus musculus
gaaaatgtgctcacccagtctccagcaatcatgtctgcaactctaggagagaaagtcaccgtgagctgcagggcccgctcaaatgtaaaatacatgtactggtaccagcagaagtcaggtgcctcccccaaactattgatttattacacatccaacctggcttctggagtcccagctcgcttcagtggcagtgggtctgggacctcttattctctcacaatcagcagcgtggaggctgaagatgctgccacttattactgccagcagtttactagctccccatttacgttcggatcggggaccaagctggaaataaaa

SEQ ID NO: 175 - DNA, 339 bp, Mus musculus
gacattgtgatgtcacagtctccatcctccctagctgtgtcagttggagagaaggttactatgagctgcaagtccagtcagaaccttttatatagtagcaatcaaaagaacttcttggcctggtaccaacagagaccagggcagtctcctaaactgctgatttactgggcatccactagggaatctggggtccctgatcgcttcacaggcagtggaactgggacagatttcactctcaccatcagcagtgtgaaggctgaagacctggcagtttattactgtcagcaatattatagatatccattcacgttcggctcggggacaaagttggaaataaaa

SEQ ID NO: 176 - DNA, 318 bp, Mus musculus
caaattgttctctcccagtctccaccaatcctgtctgcatctccaggggcgagggtcacaatgacttgcagggccagctcaagtgtgaattacatgcattggtaccagcagaagccaggatcctcccccaaaccctggatttatgccacatccaacctggcttctggagtccctgttcgcttcagtggcagtgggtctgggacctcttactctctcacaatcagcagagtggaggctgaagatgctgccacttattactgccagcagtggagtagtaacccacccacgttcggaggggggaccaagctggaaataaaa

SEQ ID NO: 177 - DNA, 321 bp, Mus musculus
gatatccagatgacacagactacatcctccctgtctgcctctctgggagacagagtcaccatcagttgcagtgcaagtcaggacattagcgattttttaaactggtatcagcagaaaccagatggaactgttaaactcctgatctattacacatcaagtttacactcaggagtcccatcaaggttcagtggcagtgggtctgggacagattattctctcaccatcaacaacctggaacctgaagatattgccacttattattgtcagcagtatagtaagcttccgtacacgttcggaggggggaccaagctggaaataaga

SEQ ID NO: 178 - DNA, 321 bp, Mus musculus
gacatccagatgactcagtctccagcctccctatctgcatctgtgggagaaactgtcaccatcacatgtcgaccaagtgagaatatttacagttatttagcatggtatcagcagaaacagggaaaatctcctcagctcctggtctataatgcaaaaaccttagtagaaggtgtgccatccaggttcagtggcagtggatcaggcacacagttttctctgaagatcaacagtgtgcagcctgaagattgtgggagttattactgtctacatcattttggtactccctggacgttcggcggaggcaccatcctggaaatcaaa

SEQ ID NO: 179 - DNA, 321 bp, Mus musculus
gatatccagatgacacagactacatcctccctgtctgcctctctgggagacagagtcaccatcagttgcagggcaagtcaggatattagcaattatttaaactggtatcagcagaaaccagatggaactgttaaactcctgatctactacacatcaagattacactcaggagtcccatcaaggttcagtggcagtgggtctgggacagattattctctcaccatcagcaacctggaacctgaagatattgccccttactattgtcagcagtatagtaagcttccgtatacgttcggatcggggaccaagctggaaataaaa

SEQ ID NO: 180 - DNA, 333 bp, Mus musculus
gatgttgtggtgactcaaactccactctccctgcctgtcagctttggagatcaagtttctatctcttgcaggtctactcagagtcttgcaaacagttatgggaacacctatttgtcttggtacctacacaagcctggccagtctccacagctcctcatctatgggatttccaacagattttctggggtgccagacaggttcagtggcagtggttcagggacagatttcacactcaagatcagcacattaaagcctgaggacttgggaatgtattactacttacaaggtacacatcagtacacattcggaggggggaccaagctggaaataaaa

SEQ ID NO: 181 - DNA, 318 bp, Mus musculus
gaaaatgtgctcacccagtctccagcaatcatgtctgcaactcttggggagaaggtcaccatgagttgcagggccagctcaaatgtaaagtacatgtactggtaccaacagaagtcaggtgcctcccccaaactctggatttattacacatccaacctgacttctggagtcccagctcgcttcagtggcagtgggtctgggacctcttattctctcacaatcagcagcgtggaggctgaggatgctgccacttattactgccagcagtttactagttccccatatacgttcggatcggggaccaaactggaaataaaa

SEQ ID NO: 182 - DNA, 333 bp, Mus musculus
gacattgtggtgacccaatctccagcttctttggctgtgtctctagggcagagggccaccatctcctgcagagccagcgaaagtattgactatgatggcgatagttatgtgaactggtaccaacagaaaccaggacagccaccgaatctcctcatctatgctgcatccaatctagaatctggcatccctgccaggtttagtggcggtgggtcggggacagacttcaccctcaccattcatcctgtggaggaggaggatgctgcaacctattactgtctgcaaagttatgagactccgtggacgttcggtggaggcaccaagctggaaatcaaa

SEQ ID NO: 183 - DNA, 321 bp, Mus musculus
gacattgtgatgacccagtctcaaaaattcgtgtccacatcagtaggagacagggtcagcgtcacctgcaaggccagtcagaatgtgggtactaatgtagtctggtatcaacagaaaccagggcaatctcctaaaccactgatttactcggcatcctaccggtacagtggagtccctgatcgcttcacaggcagtggatctgggacagatttcactctcaccatcagcaatgtgcagtctgaagacttggcagactatttctgtcaccaatataacagttctcctctcacgttcggtgttgggaccaagctggagctgaaa

SEQ ID NO: 184 - DNA, 321 bp, Mus musculus
gacatccagatgacacaatcttcatcctccttttctgtatctctaggagacagagtcaccattacttgcaaggcaagtgaggacatatataatcggttagcctggtatcagcagaaaccaggaaatgctcctaggctcttaatatctggtgcaaccactttggaaactggggttccttcaagattcagtggcagtggctctggaaaggattacgctctcagcattacaagtcttcagactgaagatgtttctacttattactgtcaagagtattggagtctttctccgacgttcggtggaggcaccaagctggaaatcaaa

SEQ ID NO: 185 - DNA, 321 bp, Mus musculus
gatatccagatgacacagactacgtcctccctgtctgcctctctgggagacagagtcaccatcagttgcagtgcaagtcaggacattagcaattatttaaactggtatcagcagaaaccagatggaactgttaaactcctgatctattacacatcaagtttacactcaggagtcccatcaaggttcagtggcagtgggtctgggacaggttattctctcaccatcagcaacctggaacctgaagatattgccacttactattgtcagcagtatagtaagcttccgtacacgttcggaggggggaccaagctggaaataaaa

SEQ ID NO: 186 - DNA, 321 bp, Mus musculus
gacattgtgatgacccagtctcaaaaattcatgtccacatcagtaggagacagggtcagcgtcacctgcaaggccagtcagaatgtgggttctaatgtagcctggtatcaacagaaatcagggcaatctcctaaagcactgatttactcggcatcctaccggtacagtggagtccctgatcgcttcacaggcagtggatatgggacagatttcactctcaccatcagcaatgtgcagtctgaagatttggcagagtatttctgtcagcaatataacaactatccgctcacgttcggtgctgggaccaagctggagctgaaa

SEQ ID NO: 187 - DNA, 333 bp, Mus musculus
gacattgtgctgacccaatctccagcttctttggctgtgactctaggacagagggccaccatatcctgcaaggccagccagagtatcaattataacggtgataattatatgcactggttccaacagaaaccaggacagccaaccaaactccttatctatactgcatccaacctagaatctggagttcctgacaggttcagtggcagtgggtctgggacagacttcaccctcaacatccatcctgtggaggaggaggatgctgcaacctattactgtcagcaaagcaaggagtttccgtggacgttcggtggaggcaccaagttggaaatcaaa

SEQ ID NO: 188 - protein, 324 aa, Mus musculus
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 189 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 190 - protein, 330 aa, Homo sapiens [sequence omitted]

SEQ ID NO: 191 - protein, 107 aa, Homo sapiens [sequence omitted]

(19) United States
(12) Patent Application Publication
(10) Pub. No.: US 2024/0059762 A1
(43) Pub. Date: Feb. 22, 2024

(54) ANTI-SARS CORONAVIRUS-2 NUCLEOCAPSID PROTEIN ANTIBODIES

(71) Applicant: Single Cell Technology, Inc., San Jose, CA (US)

(72) Inventors: Leyan Tang, San Jose, CA (US); Allison Schulkins, San Jose, CA (US); Kimberly Than, San Jose, CA (US); Chun-Nan Chen, San Jose, CA (US)

(21) Appl. No.: 18/268,192

(22) PCT Filed: Dec. 17, 2021
(86) PCT No.: PCT/US2021/064214

§ 371 Date: Jun. 16, 2023

Related U.S. Application Data
(60) Provisional application No. 63/127,902, filed on Dec. 18, 2020.

(51) Int. Cl. C07K 16/10; A61P 31/14; C12N 15/63; G01N 33/569

(52) U.S. Cl. CPC C07K 16/1003; A61P 31/14; C12N 15/63; G01N 33/56983; C07K 2317/14; C07K 2317/24; C07K 2317/40; C07K 2317/52; C07K 2317/565

(57) ABSTRACT

Provided are antibodies that specifically recognize SARS-CoV-2 N protein. Also provided are compositions comprising the antibodies and uses thereof.

### CROSS-REFERENCE TO RELATED APPLICATIONS

This application claims priority under 35 U.S.C. § 119(e) of U.S. Provisional Application No. 63/127,902, filed Dec. 18, 2020, the contents of which are incorporated by reference in its entirety into the present application.

### SEQUENCE LISTING

The instant application contains a Sequence Listing which has been submitted electronically in ASCII format and is hereby incorporated by reference in its entirety. Said ASCII copy, created on Dec. 15, 2021, is named SCT0018-101-US_Sequence_Listing_ST25.txt.

### BACKGROUND

Coronaviruses (CoV) are frequent causes of the common cold, causing upper respiratory tract infection throughout the world in all age groups (Greenberg, 2011, Crit. Care Med. 32, 433-446). In contrast, the severe acute respiratory syndrome coronavirus (SARS-CoV) and the Middle East respiratory syndrome coronavirus (MERS-CoV) are zoonotic and cause severe respiratory diseases in afflicted individuals, SARS and MERS, respectively (Fehr et al., 2017, Annu. Rev. Med. 68, 387-399). SARS emerged in 2002 in Guangdong province, China, and its subsequent global spread was associated with 8,096 cases and 774 deaths (de Wit et al., 2016, Nat. Rev. Microbiol. 14, 523-534). MERS emerged in 2012 in Jeddah, Saudi Arabia. As of Aug. 11, 2016, MERS-CoV infection has been identified in 1,791 patients, with 640 deaths (www.who.int/emergencies/mers-cov/en/). At present, no specific antivirals or approved vaccines are available to combat SARS nor MERS, and the SARS pandemic in 2002 and 2003 was finally stopped by conventional control measures and so was the MERS outbreaks.

In December 2019, a new infectious respiratory disease emerged in Wuhan, Hubei province, China (Huang et al., 2020, The Lancet 395, 497-506; Wang et al., 2020, The Lancet 395, 470-473; and Zhu et al., 2020, N. Engl. J. Med). An initial cluster of infections was linked to Huanan seafood market, potentially due to animal contact. Subsequently, human-to-human transmission occurred (Chan et al., 2020, The Lancet 395, 514-523) and the disease, now termed coronavirus disease 19 (COVID-19) rapidly spread within China. A novel coronavirus, SARS-coronavirus 2 (SARS-CoV-2), which is closely related to SARS-CoV, was detected in patients and is believed to be the etiologic agent of COVID-19 (Zhu et al., 2020). Several factors, including symptoms similar to common cold, government and public not taking the spread seriously, and presymptomatic transmission (Wei, 2020, Presymptomatic Transmission of SARS-CoV-2—Singapore, Jan. 23-Mar. 16, 2020. MMWR Morb. Mortal. Wkly. Rep. 69), among others, contributed to the rapid transmission of COVID-19 throughout the world. As of Dec. 10, 2020, there are 68,165,877 confirmed cases and 1,557,385 associated deaths worldwide (covid19.who.int).

To implement an effective epidemiological control of the transmission, there is an urgent need for a fast and sensitive detection of SARS-CoV-2 virus especially in presymptomatic or asymptomatic patients. Having available high affinity, high specificity anti-SARS-CoV-2 protein (such as anti-SARS-CoV-2 N protein) antibodies will enable such a goal. This disclosure satisfies these needs and provides related advantages as well.

### SUMMARY

Provided are antibodies and fragments thereof that recognize and specifically bind to SARS-CoV-2 protein such as the N protein. In some embodiments, the antibodies or fragments thereof as disclosed herein neutralize a SARS-CoV-2, and accordingly, are useful to diagnose or treat a subject having or suspected of having coronavirus disease 2019 (COVID-19) infection. In other embodiments, the antibodies or fragments thereof can also be used to detect a SARS-CoV-2 and thus, are used as a diagnostic tool. Other suitable uses of the antibodies are also disclosed herein, such as for in vitro or in vivo screening another clinical or diagnostic candidate that in one aspect, may competitively bind to the SARS-CoV-2. Yet further, the antibodies and fragments thereof can be combined with other suitable therapies to treat and prevent infection. In one embodiment, the disclosure provides an antibody and fragments thereof that bind to SEQ ID NO:1.

In one aspect, provided is an antibody or antigen binding fragment thereof. The antibody or antigen binding fragment thereof comprises, or consists essentially of, or yet further consists of one or more of:
(SCT-Pa01) a heavy chain (HC) complementarity determining region (CDR) 1 (HCDR1) that comprises, or consists essentially of, or yet further consists of the amino acid sequence of SEQ ID NO: 44, an HC CDR 2 (HCDR2) that comprises, or consists essentially of, or yet further consists of the amino acid sequence of SEQ ID NO: 45, an HC CDR 3 (HCDR3) that comprises, or consists essentially of, or yet further consists of the amino acid sequence of SEQ ID NO: 46, a light chain (LC) complementarity determining region (CDR) 1 (LCDR1) that comprises, or consists essentially of, or yet further consists of the amino acid sequence of SEQ ID NO: 100, an LC CDR 2 (LCDR2) that comprises, or consists essentially of, or yet further consists of the amino acid sequence of SEQ ID NO: 101, and an LC CDR 3 (LCDR3) that comprises, or consists essentially of, or yet further consists of the amino acid sequence of SEQ ID NO: 102;
(SCT-Pa02) an HCDR1 that comprises, or consists essentially of, or yet further consists of the amino acid sequence of SEQ ID NO: 47, an HCDR2 that comprises, or consists essentially of, or yet further consists of the amino acid sequence of SEQ ID NO: 48, an HCDR3 that comprises, or consists essentially of, or yet further consists of the amino acid sequence of SEQ ID NO: 49, an LCDR1 that comprises, or consists essentially of, or yet further consists of the amino acid sequence of SEQ ID NO: 103, an LCDR2 that comprises, or consists essentially of, or yet further consists of the amino acid sequence of SEQ ID NO: 104, and an LCDR3 that comprises, or consists essentially of, or yet further consists of the amino acid sequence of SEQ ID NO: 105;
(SCT-Pa03) an HCDR1 that comprises, or consists essentially of, or yet further consists of the amino acid sequence of SEQ ID NO: 50, an HCDR2 that comprises, or consists essentially of, or yet further consists of the amino acid sequence of SEQ ID NO: 51, an HCDR3 that comprises, or consists essentially of, or yet further consists of the amino acid sequence of SEQ ID NO: 52, an LCDR1 that comprises, or consists essentially of, or yet further consists of the amino acid sequence of SEQ ID NO: 106, an LCDR2 that comprises, or consists essentially of, or yet further consists of the amino acid sequence of SEQ ID NO: 107, and an LCDR3 that comprises, or consists essentially of, or yet further consists of the amino acid sequence of SEQ ID NO: 108;
(SCT-Pa04) an HCDR1 that comprises, or consists essentially of, or yet further consists of the amino acid sequence of SEQ ID NO: 53, an HCDR2 that comprises, or consists essentially of, or yet further consists of the amino acid sequence of SEQ ID NO: 54, an HCDR3 that comprises, or consists essentially of, or yet further consists of the amino acid sequence of SEQ ID NO: 55, an LCDR1 that comprises, or consists essentially of, or yet further consists of the amino acid sequence of SEQ ID NO: 109, an LCDR2 that comprises, or consists essentially of, or yet further consists of the amino acid sequence of SEQ ID NO: 110, and an LCDR3 that comprises, or consists essentially of, or yet further consists of the amino acid sequence of SEQ ID NO: 111;
(SCT-Pa05) an HCDR1 that comprises, or consists essentially of, or yet further consists of the amino acid sequence of SEQ ID NO: 56, an HCDR2 that comprises, or consists essentially of, or yet further consists of the amino acid sequence of SEQ ID NO: 57, an HCDR3 that comprises, or consists essentially of, or yet further consists of the amino acid sequence of SEQ ID NO: 58, an LCDR1 that comprises, or consists essentially of, or yet further consists of the amino acid sequence of SEQ ID NO: 112, an LCDR2 that comprises, or consists essentially of, or yet further consists of the amino acid sequence of SEQ ID NO: 113, and an LCDR3 that comprises, or consists essentially of, or yet further consists of the amino acid sequence of SEQ ID NO: 114;
(SCT-Pa06) an HCDR1 that comprises, or consists essentially of, or yet further consists of the amino acid sequence of SEQ ID NO: 59, an HCDR2 that comprises, or consists essentially of, or yet further consists of the amino acid sequence of SEQ ID NO: 60, an HCDR3 that comprises, or consists essentially of, or yet further consists of the amino acid sequence of SEQ ID NO: 61, an LCDR1 that comprises, or consists essentially of, or yet further consists of the amino acid sequence of SEQ ID NO: 115, an LCDR2 that comprises, or consists essentially of, or yet further consists of the amino acid sequence of SEQ ID NO: 101, and an LCDR3 that comprises, or consists essentially of, or yet further consists of the amino acid sequence of SEQ ID NO: 116;
(SCT-Pa07) an HCDR1 that comprises, or consists essentially of, or yet further consists of the amino acid sequence of SEQ ID NO: 50, an HCDR2 that comprises, or consists essentially of, or yet further consists of the amino acid sequence of SEQ ID NO: 62, an HCDR3 that comprises, or consists essentially of, or yet further consists of the amino acid sequence of SEQ ID NO: 63, an LCDR1 that comprises, or consists essentially of, or yet further consists of the amino acid sequence of SEQ ID NO: 117, an LCDR2 that comprises, or consists essentially of, or yet further consists of the amino acid sequence of SEQ ID NO: 118, and an LCDR3 that comprises, or consists essentially of, or yet further consists of the amino acid sequence of SEQ ID NO: 119;
(SCT-Pa08) an HCDR1 that comprises, or consists essentially of, or yet further consists of the amino acid sequence of SEQ ID NO: 64, an HCDR2 that comprises, or consists essentially of, or yet further consists of the amino acid sequence of SEQ ID NO: 65, an HCDR3 that comprises, or consists essentially of, or yet further consists of the amino acid sequence of SEQ ID NO: 66, an LCDR1 that comprises, or consists essentially of, or yet further consists of the amino acid sequence of SEQ ID NO: 120, an LCDR2 that comprises, or consists essentially of, or yet further consists of the amino acid sequence of SEQ ID NO: 121, and an LCDR3 that comprises, or consists essentially of, or yet further consists of the amino acid sequence of SEQ ID NO: 122;
(SCT-Pa09) an HCDR1 that comprises, or consists essentially of, or yet further consists of the amino acid sequence of SEQ ID NO: 67, an HCDR2 that comprises, or consists essentially of, or yet further consists of the amino acid sequence of SEQ ID NO: 68, an HCDR3 that comprises, or consists essentially of, or yet further consists of the amino acid sequence of SEQ ID NO: 69, an LCDR1 that comprises, or consists essentially of, or yet further consists of the amino acid sequence of SEQ ID NO: 123, an LCDR2 that comprises, or consists essentially of, or yet further consists of the amino acid sequence of SEQ ID NO: 104, and an LCDR3 that comprises, or consists essentially of, or yet further consists of the amino acid sequence of SEQ ID NO: 124;
(SCT-Pa10) an HCDR1 that comprises, or consists essentially of, or yet further consists of the amino acid sequence of SEQ ID NO: 70, an HCDR2 that comprises, or consists essentially of, or yet further consists of the amino acid sequence of SEQ ID NO: 71, an HCDR3 that comprises, or consists essentially of, or yet further consists of the amino acid sequence of SEQ ID NO: 72, an LCDR1 that comprises, or consists essentially of, or yet further consists of the amino acid sequence of SEQ ID NO: 125, an LCDR2 that comprises, or consists essentially of, or yet further consists of the amino acid sequence of SEQ ID NO: 118, and an LCDR3 that comprises, or consists essentially of, or yet further consists of the amino acid sequence of SEQ ID NO: 126;
(SCT-Pa11) an HCDR1 that comprises, or consists essentially of, or yet further consists of the amino acid sequence of SEQ ID NO: 73, an HCDR2 that comprises, or consists essentially of, or yet further consists of the amino acid sequence of SEQ ID NO: 54, an HCDR3 that comprises, or consists essentially of, or yet further consists of the amino acid sequence of SEQ ID NO: 74, an LCDR1 that comprises, or consists essentially of, or yet further consists of the amino acid sequence of SEQ ID NO: 127, an LCDR2 that comprises, or consists essentially of, or yet further consists of the amino acid sequence of SEQ ID NO: 128, and an LCDR3 that comprises, or consists essentially of, or yet further consists of the amino acid sequence of SEQ ID NO: 129;
(SCT-Pa12) an HCDR1 that comprises, or consists essentially of, or yet further consists of the amino acid sequence of SEQ ID NO: 75, an HCDR2 that comprises, or consists essentially of, or yet further consists of the amino acid sequence of SEQ ID NO: 76, an HCDR3 that comprises, or consists essentially of, or yet further consists of the amino acid sequence of SEQ ID NO: 77, an LCDR1 that comprises, or consists essentially of, or yet further consists of the amino acid sequence of SEQ ID NO: 130, an LCDR2 that comprises, or consists essentially of, or yet further consists of the amino acid sequence of SEQ ID NO: 118, and an LCDR3 that comprises, or consists essentially of, or yet further consists of the amino acid sequence of SEQ ID NO: 126;
(SCT-Pa13) an HCDR1 that comprises, or consists essentially of, or yet further consists of the amino acid sequence of SEQ ID NO: 78, an HCDR2 that comprises, or consists essentially of, or yet further consists of the amino acid sequence of SEQ ID NO: 79, an HCDR3 that comprises, or consists essentially of, or yet further consists of the amino acid sequence of SEQ ID NO: 80, an LCDR1 that comprises, or consists essentially of, or yet further consists of the amino acid sequence of SEQ ID NO: 131, an LCDR2 that comprises, or consists essentially of, or yet further consists of the amino acid sequence of SEQ ID NO: 132, and an LCDR3 that comprises, or consists essentially of, or yet further consists of the amino acid sequence of SEQ ID NO: 133;
(SCT-Pa14) an HCDR1 that comprises, or consists essentially of, or yet further consists of the amino acid sequence of SEQ ID NO: 81, an HCDR2 that comprises, or consists essentially of, or yet further consists of the amino acid sequence of SEQ ID NO: 82, an HCDR3 that comprises, or consists essentially of, or yet further consists of the amino acid sequence of SEQ ID NO: 83, an LCDR1 that comprises, or consists essentially of, or yet further consists of the amino acid sequence of SEQ ID NO: 117, an LCDR2 that comprises, or consists essentially of, or yet further consists of the amino acid sequence of SEQ ID NO: 118, and an LCDR3 that comprises, or consists essentially of, or yet further consists of the amino acid sequence of SEQ ID NO: 134;
(SCT-Pa15) an HCDR1 that comprises, or consists essentially of, or yet further consists of the amino acid sequence of SEQ ID NO: 84, an HCDR2 that comprises, or consists essentially of, or yet further consists of the amino acid sequence of SEQ ID NO: 79, an HCDR3 that comprises, or consists essentially of, or yet further consists of the amino acid sequence of SEQ ID NO: 85, an LCDR1 that comprises, or consists essentially of, or yet further consists of the amino acid sequence of SEQ ID NO: 135, an LCDR2 that comprises, or consists essentially of, or yet further consists of the amino acid sequence of SEQ ID NO: 136, and an LCDR3 that comprises, or consists essentially of, or yet further consists of the amino acid sequence of SEQ ID NO: 137;
(SCT-Pa16) an HCDR1 that comprises, or consists essentially of, or yet further consists of the amino acid sequence of SEQ ID NO: 86, an HCDR2 that comprises, or consists essentially of, or yet further consists of the amino acid sequence of SEQ ID NO: 87, an HCDR3 that comprises, or consists essentially of, or yet further consists of the amino acid sequence of SEQ ID NO: 88, an LCDR1 that comprises, or consists essentially of, or yet further consists of the amino acid sequence of SEQ ID NO: 138, an LCDR2 that comprises, or consists essentially of, or yet further consists of the amino acid sequence of SEQ ID NO: 139, and an LCDR3 that comprises, or consists essentially of, or yet further consists of the amino acid sequence of SEQ ID NO: 140;
(SCT-Pa17) an HCDR1 that comprises, or consists essentially of, or yet further consists of the amino acid sequence of SEQ ID NO: 89, an HCDR2 that comprises, or consists essentially of, or yet further consists of the amino acid sequence of SEQ ID NO: 90, an HCDR3 that comprises, or consists essentially of, or yet further consists of the amino acid sequence of SEQ ID NO: 91, an LCDR1 that comprises, or consists essentially of, or yet further consists of the amino acid sequence of SEQ ID NO: 141, an LCDR2 that comprises, or consists essentially of, or yet further consists of the amino acid sequence of SEQ ID NO: 107, and an LCDR3 that comprises, or consists essentially of, or yet further consists of the amino acid sequence of SEQ ID NO: 142;
(SCT-Pa18) an HCDR1 that comprises, or consists essentially of, or yet further consists of the amino acid sequence of SEQ ID NO: 86, an HCDR2 that comprises, or consists essentially of, or yet further consists of the amino acid sequence of SEQ ID NO: 92, an HCDR3 that comprises, or consists essentially of, or yet further consists of the amino acid sequence of SEQ ID NO: 93, an LCDR1 that comprises, or consists essentially of, or yet further consists of the amino acid sequence of SEQ ID NO: 130, an LCDR2 that comprises, or consists essentially of, or yet further consists of the amino acid sequence of SEQ ID NO: 118, and an LCDR3 that comprises, or consists essentially of, or yet further consists of the amino acid sequence of SEQ ID NO: 126;
(SCT-Pa19) an HCDR1 that comprises, or consists essentially of, or yet further consists of the amino acid sequence of SEQ ID NO: 94, an HCDR2 that comprises, or consists essentially of, or yet further consists of the amino acid sequence of SEQ ID NO: 95, an HCDR3 that comprises, or consists essentially of, or yet further consists of the amino acid sequence of SEQ ID NO: 96, an LCDR1 that comprises, or consists essentially of, or yet further consists of the amino acid sequence of SEQ ID NO: 143, an LCDR2 that comprises, or consists essentially of, or yet further consists of the amino acid sequence of SEQ ID NO: 139, and an LCDR3 that comprises, or consists essentially of, or yet further consists of the amino acid sequence of SEQ ID NO: 144; or
(SCT-Pa20) an HCDR1 that comprises, or consists essentially of, or yet further consists of the amino acid sequence of SEQ ID NO: 97, an HCDR2 that comprises, or consists essentially of, or yet further consists of the amino acid sequence of SEQ ID NO: 98, an HCDR3 that comprises, or consists essentially of, or yet further consists of the amino acid sequence of SEQ ID NO: 99, an LCDR1 that comprises, or consists essentially of, or yet further consists of the amino acid sequence of SEQ ID NO: 145, an LCDR2 that comprises, or consists essentially of, or yet further consists of the amino acid sequence of SEQ ID NO: 146, and an LCDR3 that comprises, or consists essentially of, or yet further consists of the amino acid sequence of SEQ ID NO: 147.

Additionally or alternatively, the antibody or antigen binding fragment comprises, or consists essentially of, or yet further consists of:
(SCT-Pa01) a heavy chain variable domain that comprises, or consists essentially of, or yet further consists of SEQ ID NO: 4 and a light chain variable domain that comprises, or consists essentially of, or yet further consists of SEQ ID NO: 24, or an equivalent of each thereof comprising CDRs of the variable domain;
(SCT-Pa02) a heavy chain variable domain that comprises, or consists essentially of, or yet further consists of SEQ ID NO: 5 and a light chain variable domain that comprises, or consists essentially of, or yet further consists of SEQ ID NO: 25, or an equivalent of each thereof comprising CDRs of the variable domain;
(SCT-Pa03) a heavy chain variable domain that comprises, or consists essentially of, or yet further consists of SEQ ID NO: 6 and a light chain variable domain that comprises, or consists essentially of, or yet further consists of SEQ ID NO: 26, or an equivalent of each thereof comprising CDRs of the variable domain;
(SCT-Pa04) a heavy chain variable domain that comprises, or consists essentially of, or yet further consists of SEQ ID NO: 7 and a light chain variable domain that comprises, or consists essentially of, or yet further consists of SEQ ID NO: 27, or an equivalent of each thereof comprising CDRs of the variable domain;
(SCT-Pa05) a heavy chain variable domain that comprises, or consists essentially of, or yet further consists of SEQ ID NO: 8 and a light chain variable domain that comprises, or consists essentially of, or yet further consists of SEQ ID NO: 28, or an equivalent of each thereof comprising CDRs of the variable domain;
(SCT-Pa06) a heavy chain variable domain that comprises, or consists essentially of, or yet further consists of SEQ ID NO: 9 and a light chain variable domain that comprises, or consists essentially of, or yet further consists of SEQ ID NO: 29, or an equivalent of each thereof comprising CDRs of the variable domain;
(SCT-Pa07) a heavy chain variable domain that comprises, or consists essentially of, or yet further consists of SEQ ID NO: 10 and a light chain variable domain that comprises, or consists essentially of, or yet further consists of SEQ ID NO: 30, or an equivalent of each thereof comprising CDRs of the variable domain;
(SCT-Pa08) a heavy chain variable domain that comprises, or consists essentially of, or yet further consists of SEQ ID NO: 11 and a light chain variable domain that comprises, or consists essentially of, or yet further consists of SEQ ID NO: 31, or an equivalent of each thereof comprising CDRs of the variable domain;
(SCT-Pa09) a heavy chain variable domain that comprises, or consists essentially of, or yet further consists of SEQ ID NO: 12 and a light chain variable domain that comprises, or consists essentially of, or yet further consists of SEQ ID NO: 32, or an equivalent of each thereof comprising CDRs of the variable domain;
(SCT-Pa10) a heavy chain variable domain that comprises, or consists essentially of, or yet further consists of SEQ ID NO: 13 and a light chain variable domain that comprises, or consists essentially of, or yet further consists of SEQ ID NO: 33, or an equivalent of each thereof comprising CDRs of the variable domain;
(SCT-Pa11) a heavy chain variable domain that comprises, or consists essentially of, or yet further consists of SEQ ID NO: 14 and a light chain variable domain that comprises, or consists essentially of, or yet further consists of SEQ ID NO: 34, or an equivalent of each thereof comprising CDRs of the variable domain;
(SCT-Pa12) a heavy chain variable domain that comprises, or consists essentially of, or yet further consists of SEQ ID NO: 15 and a light chain variable domain that comprises, or consists essentially of, or yet further consists of SEQ ID NO: 35, or an equivalent of each thereof comprising CDRs of the variable domain;
(SCT-Pa13) a heavy chain variable domain that comprises, or consists essentially of, or yet further consists of SEQ ID NO: 16 and a light chain variable domain that comprises, or consists essentially of, or yet further consists of SEQ ID NO: 36, or an equivalent of each thereof comprising CDRs of the variable domain;
(SCT-Pa14) a heavy chain variable domain that comprises, or consists essentially of, or yet further consists of SEQ ID NO: 17 and a light chain variable domain that comprises, or consists essentially of, or yet further consists of SEQ ID NO: 37, or an equivalent of each thereof comprising CDRs of the variable domain;
(SCT-Pa15) a heavy chain variable domain that comprises, or consists essentially of, or yet further consists of SEQ ID NO: 18 and a light chain variable domain that comprises, or consists essentially of, or yet further consists of SEQ ID NO: 38, or an equivalent of each thereof comprising CDRs of the variable domain;
(SCT-Pa16) a heavy chain variable domain that comprises, or consists essentially of, or yet further consists of SEQ ID NO: 19 and a light chain variable domain that comprises, or consists essentially of, or yet further consists of SEQ ID NO: 39, or an equivalent of each thereof comprising CDRs of the variable domain;
(SCT-Pa17) a heavy chain variable domain that comprises, or consists essentially of, or yet further consists of SEQ ID NO: 20 and a light chain variable domain that comprises, or consists essentially of, or yet further consists of SEQ ID NO: 40, or an equivalent of each thereof comprising CDRs of the variable domain;
(SCT-Pa18) a heavy chain variable domain that comprises, or consists essentially of, or yet further consists of SEQ ID NO: 21 and a light chain variable domain that comprises, or consists essentially of, or yet further consists of SEQ ID NO: 41, or an equivalent of each thereof comprising CDRs of the variable domain;
(SCT-Pa19) a heavy chain variable domain that comprises, or consists essentially of, or yet further consists of SEQ ID NO: 22 and a light chain variable domain that comprises, or consists essentially of, or yet further consists of SEQ ID NO: 42, or an equivalent of each thereof comprising CDRs of the variable domain; or
(SCT-Pa20) a heavy chain variable domain that comprises, or consists essentially of, or yet further consists of SEQ ID NO: 23 and a light chain variable domain that comprises, or consists essentially of, or yet further consists of SEQ ID NO: 43, or an equivalent of each thereof comprising CDRs of the variable domain.

In some embodiments, the antibody or antigen binding fragment as disclosed herein specifically recognizes and binds to a nucleocapsid (N) protein of severe acute respiratory syndrome coronavirus 2 (SARS-CoV-2) or an immunogenic fragment thereof. In further embodiments, the N protein comprises, or consists essentially of, or yet further consists of the amino acid sequence of SEQ ID NO: 1.

In a further aspect, provided is a polypeptide comprising, or consisting essentially of, or yet further consisting of an antibody or antigen binding fragment as disclosed herein. In yet a further aspect, provided is a polypeptide comprising, or consisting essentially of, or yet further consisting of any one or more of the variable domain as disclosed herein.

In one aspect, provided is a polynucleotide encoding the antibody or antigen binding fragment or polypeptide as disclosed herein, or a polynucleotide complementary thereto. In another aspect, provided is a vector comprising, or consisting essentially of, or yet further consisting of the polynucleotide as disclosed herein. In yet another aspect, provided is a cell comprising one or more of: an antibody or antigen binding fragment as disclosed herein, a polypeptide as disclosed herein, a polynucleotide as disclosed herein, or a vector as disclosed herein. Additionally provided is a hybridoma expressing an antibody or antigen binding fragment as disclosed herein.

In one aspect, provided is a method of producing one or more of an antibody or antigen binding fragment, polypeptides comprising the antibody or antigen binding fragment, CDRs, heavy chain, or light chain, as disclosed herein. In some embodiments, the method comprises, or consists essentially of, or yet further consists of culturing a cell comprising a polynucleotide encoding the antibody or the antigen binding fragment, polypeptides comprising the antibody or antigen binding fragment, CDRs, heavy chain, or light chain, under conditions suitable for expression of the antibody or antigen binding fragment, polypeptides comprising the antibody or antigen binding fragment, CDRs, heavy chain, or light chain as disclosed herein. Additionally or alternatively, the method comprises, or consists essentially of, or yet further consists of culturing a hybridoma as disclosed herein under conditions suitable for expression of the antibody or antigen binding fragment. In some embodiments, the method comprises, or consists essentially of, or yet further consists of contacting a polynucleotide as disclosed herein or a vector as disclosed herein with an RNA polymerase, adenosine triphosphate (ATP), cytidine triphosphate (CTP), guanosine-5′-triphosphate (GTP), and uridine triphosphate (UTP) under conditions suitable for transcription to messenger RNA, and contacting the transcribed messenger RNA with a ribosome, tRNAs, an aminoacyl-tRNA synthetase, and initiation, elongation and termination factors under conditions suitable for translation to the antibody or antigen binding fragment.

In another aspect, provided is a composition comprising, or consisting essentially of, or yet further consisting of a carrier (such as a pharmaceutically acceptable carrier) and one or more of: an antibody or antigen binding fragment as disclosed herein, a polypeptide as disclosed herein, a polynucleotide as disclosed herein, a vector as disclosed herein, a cell as disclosed herein, or a hybridoma as disclosed herein.

In yet another aspect, provided is a method of one or more of: (a) treating a subject having or suspect of having or at risk of a SARS-CoV-2 infection, (b) conferring anti-SARS-CoV-2 passive immunity to a subject in need thereof, (c) conferring or inducing an immune response to SARS-CoV-2 in a subject in need thereof, (d) neutralizing SARS-CoV-2 in a subject in need thereof, or (e) decreasing viral load of SARS-CoV-2 in a subject. The method comprises, or consists essentially of, or yet further consists of administering to the subject, optionally an effective amount of, one or more of: an antibody or antigen binding fragment as disclosed herein, a polypeptide as disclosed herein, a polynucleotide as disclosed herein, a vector as disclosed herein, or a cell as disclosed herein.

In one aspect, provided is a detection system comprising, or consisting essentially of, or yet further consisting of an antibody or antigen binding fragment as disclosed herein. In one aspect the system also contains a detectable marker that produces a detectable signal upon binding of the antibody or antigen binding fragment thereof with a SARS-CoV-2 N protein or an immunogenic fragment thereof. In some embodiments, the system is an enzyme-linked immunosorbent assay (ELISA) or a lateral flow immunoassay.

In a further aspect, provided is a method comprising, or consisting essentially of, or yet further consisting of contacting the antibody or antigen binding fragment of the detection system with a biological sample isolated from a subject. In some embodiments, the binding of the antibody or antigen binding fragment thereof with a component of the biological sample indicates the subject has or had a SARS-CoV-2 infection.

Additionally provided is a kit for use in a method as disclosed herein.

### BRIEF DESCRIPTION OF THE DRAWINGS

FIG. 1 Epitope bins of anti-SARS-CoV-2 N protein mAbs by BLI assays. Epitope binning of 17 recombinant anti-SARS-CoV-2 N protein mAbs. Recombinant mAbs blocking each other's binding to SARS-CoV-2 N protein are grouped by solid lines into seven distinct epitope bins.

### BRIEF DESCRIPTION OF EXEMPLARY SEQUENCES

TABLE 1
Table of Exemplary Sequences.

| SEQ | |
|---|---|
| ID | |
| NO. | Description of sequence |
| 1 | SARS-CoV-2 N protein sequence |
| 2 | SARS-CoV-2 N protein His tagged protein sequence |
| 3 | Human TNFR2 protein His tagged protein sequence |
| 4 | SCT-Pa01 mature heavy chain variable domain protein sequence |
| 5 | SCT-Pa02 mature heavy chain variable domain protein sequence |
| 6 | SCT-Pa03 mature heavy chain variable domain protein sequence |
| 7 | SCT-Pa04 mature heavy chain variable domain protein sequence |
| 8 | SCT-Pa05 mature heavy chain variable domain protein sequence |
| 9 | SCT-Pa06 mature heavy chain variable domain protein sequence |
| 10 | SCT-Pa07 mature heavy chain variable domain protein sequence |
| 11 | SCT-Pa08 mature heavy chain variable domain protein sequence |
| 12 | SCT-Pa09 mature heavy chain variable domain protein sequence |
| 13 | SCT-Pa10 mature heavy chain variable domain protein sequence |
| 14 | SCT-Pa11 mature heavy chain variable domain protein sequence |
| 15 | SCT-Pa12 mature heavy chain variable domain protein sequence |
| 16 | SCT-Pa13 mature heavy chain variable domain protein sequence |
| 17 | SCT-Pa14 mature heavy chain variable domain protein sequence |
| 18 | SCT-Pa15 mature heavy chain variable domain protein sequence |
| 19 | SCT-Pa16 mature heavy chain variable domain protein sequence |
| 20 | SCT-Pa17 mature heavy chain variable domain protein sequence |
| 21 | SCT-Pa18 mature heavy chain variable domain protein sequence |
| 22 | SCT-Pa19 mature heavy chain variable domain protein sequence |
| 23 | SCT-Pa20 mature heavy chain variable domain protein sequence |
| 24 | SCT-Pa01 mature light chain variable domain protein sequence |
| 25 | SCT-Pa02 mature light chain variable domain protein sequence |
| 26 | SCT-Pa03 mature light chain variable domain protein sequence |
| 27 | SCT-Pa04 mature light chain variable domain protein sequence |
| 28 | SCT-Pa05 mature light chain variable domain protein sequence |
| 29 | SCT-Pa06 mature light chain variable domain protein sequence |
| 30 | SCT-Pa07 mature light chain variable domain protein sequence |
| 31 | SCT-Pa08 mature light chain variable domain protein sequence |
| 32 | SCT-Pa09 mature light chain variable domain protein sequence |
| 33 | SCT-Pa10 mature light chain variable domain protein sequence |
| 34 | SCT-Pa11 mature light chain variable domain protein sequence |
| 35 | SCT-Pa12 mature light chain variable domain protein sequence |
| 36 | SCT-Pa13 mature light chain variable domain protein sequence |
| 37 | SCT-Pa14 mature light chain variable domain protein sequence |
| 38 | SCT-Pa15 mature light chain variable domain protein sequence |
| 39 | SCT-Pa16 mature light chain variable domain protein sequence |
| 40 | SCT-Pa17 mature light chain variable domain protein sequence |
| 41 | SCT-Pa18 mature light chain variable domain protein sequence |
| 42 | SCT-Pa19 mature light chain variable domain protein sequence |
| 43 | SCT-Pa20 mature light chain variable domain protein sequence |
| 188 | Mouse IgG1 heavy chain constant region |
| 189 | Mouse kappa light chain constant region |
| 148 | SCT-Pa01 mature heavy chain variable domain encoding |
| | DNA sequence |
| 149 | SCT-Pa02 mature heavy chain variable domain encoding |
| | DNA sequence |
| 150 | SCT-Pa03 mature heavy chain variable domain encoding |
| | DNA sequence |
| 151 | SCT-Pa04 mature heavy chain variable domain encoding |
| | DNA sequence |
| 152 | SCT-Pa05 mature heavy chain variable domain encoding |
| | DNA sequence |
| 153 | SCT-Pa06 mature heavy chain variable domain encoding |
| | DNA sequence |
| 154 | SCT-Pa07 mature heavy chain variable domain encoding |
| | DNA sequence |
| 155 | SCT-Pa08 mature heavy chain variable domain encoding |
| | DNA sequence |
| 156 | SCT-Pa09 mature heavy chain variable domain encoding |
| | DNA sequence |
| 157 | SCT-Pa10 mature heavy chain variable domain encoding |
| | DNA sequence |
| 158 | SCT-Pa11 mature heavy chain variable domain encoding |
| | DNA sequence |
| 159 | SCT-Pa12 mature heavy chain variable domain encoding |
| | DNA sequence |
| 160 | SCT-Pa13 mature heavy chain variable domain encoding |
| | DNA sequence |
| 161 | SCT-Pa14 mature heavy chain variable domain encoding |
| | DNA sequence |
| 162 | SCT-Pa15 mature heavy chain variable domain encoding |
| | DNA sequence |
| 163 | SCT-Pa16 mature heavy chain variable domain encoding |
| | DNA sequence |
| 164 | SCT-Pa17 mature heavy chain variable domain encoding |
| | DNA sequence |
| 165 | SCT-Pa18 mature heavy chain variable domain encoding |
| | DNA sequence |
| 166 | SCT-Pa19 mature heavy chain variable domain encoding |
| | DNA sequence |
| 167 | SCT-Pa20 mature heavy chain variable domain encoding |
| | DNA sequence |
| 168 | SCT-Pa01 mature light chain variable domain encoding |
| | DNA sequence |
| 169 | SCT-Pa02 mature light chain variable domain encoding |
| | DNA sequence |
| 170 | SCT-Pa03 mature light chain variable domain encoding |
| | DNA sequence |
| 171 | SCT-Pa04 mature light chain variable domain encoding |
| | DNA sequence |
| 172 | SCT-Pa05 mature light chain variable domain encoding |
| | DNA sequence |
| 173 | SCT-Pa06 mature light chain variable domain encoding |
| | DNA sequence |
| 174 | SCT-Pa07 mature light chain variable domain encoding |
| | DNA sequence |
| 175 | SCT-Pa08 mature light chain variable domain encoding |
| | DNA sequence |
| 176 | SCT-Pa09 mature light chain variable domain encoding |
| | DNA sequence |
| 177 | SCT-Pa10 mature light chain variable domain encoding |
| | DNA sequence |
| 178 | SCT-Pa11 mature light chain variable domain encoding |
| | DNA sequence |
| 179 | SCT-Pa12 mature light chain variable domain encoding |
| | DNA sequence |
| 180 | SCT-Pa13 mature light chain variable domain encoding |
| | DNA sequence |
| 181 | SCT-Pa14 mature light chain variable domain encoding |
| | DNA sequence |
| 182 | SCT-Pa15 mature light chain variable domain encoding |
| | DNA sequence |
| 183 | SCT-Pa16 mature light chain variable domain encoding |
| | DNA sequence |
| 184 | SCT-Pa17 mature light chain variable domain encoding |
| | DNA sequence |
| 185 | SCT-Pa18 mature light chain variable domain encoding |
| | DNA sequence |
| 186 | SCT-Pa19 mature light chain variable domain encoding |
| | DNA sequence |
| 187 | SCT-Pa20 mature light chain variable domain encoding |
| | DNA sequence |
| 190 | Human IgG1 heavy chain constant region |
| 191 | Human kappa light chain constant region |

### DETAILED DESCRIPTION

### Definitions

As it would be understood, the section or subsection headings as used herein is for organizational purposes only and are not to be construed as limiting and/or separating the subject matter described.

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Techique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

As will be understood by one skilled in the art, for any and all purposes, all ranges disclosed herein also encompass any and all possible subranges and combinations of subranges thereof. Furthermore, as will be understood by one skilled in the art, a range includes each individual member.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

The phrase “therapeutically effective” is intended to qualify the amount of active ingredients used in the treatment of a disease or disorder or on the effecting of a clinical endpoint. In some embodiments, an antibody or antigen binding fragment thereof is administered to a subject in a therapeutically effective amount.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use. In some embodiments, an antibody or antigen binding fragment thereof is administered to a subject in a therapeutically acceptable amount.

In some embodiments, the terms “first” “second” “third” “fourth” or similar in a component name are used to distinguish and identify more than one components sharing certain identity in their names. For example, “first antibody” and “second antibody” are used to distinguishing two antibodies.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention. As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

As used herein, “complementary” sequences refer to two nucleotide sequences which, when aligned anti-parallel to each other, contain multiple individual nucleotide bases which pair with each other. Paring of nucleotide bases forms hydrogen bonds and thus stabilizes the double strand structure formed by the complementary sequences. It is not necessary for every nucleotide base in two sequences to pair with each other for sequences to be considered “complementary”. Sequences may be considered complementary, for example, if at least 30%, 40%, 50%, 55%, 60%, 65%, 70%, 75%, 80%, 85%, 90%, 95%, 98%, 99%, or 100% of the nucleotide bases in two sequences pair with each other. In some embodiments, the term complementary refers to 100% of the nucleotide bases in two sequences pair with each other. In addition, sequences may still be considered “complementary” when the total lengths of the two sequences are significantly different from each other. For example, a primer of 15 nucleotides may be considered “complementary” to a longer polynucleotide containing hundreds of nucleotides if multiple individual nucleotide bases of the primer pair with nucleotide bases in the longer polynucleotide when the primer is aligned anti-parallel to a particular region of the longer polynucleotide. Nucleotide bases paring is known in the field, such as in DNA, the purine adenine (A) pairs with the pyrimidine thymine (T) and the pyrimidine cytosine (C) always pairs with the purine guanine (G); while in RNA, adenine (A) pairs with uracil (U) and guanine (G) pairs with cytosine (C). Further, the nucleotide bases aligned anti-parallel to each other in two complementary sequences, but not a pair, are referred to herein as a mismatch.

A “gene” refers to a polynucleotide containing at least one open reading frame (ORF) that is capable of encoding a particular polypeptide or protein after being transcribed and translated.

The term “express” refers to the production of a gene product, such as mRNA, peptides, polypeptides or proteins. As used herein, “expression” refers to the process by which polynucleotides are transcribed into mRNA or the process by which the transcribed mRNA is subsequently being translated into peptides, polypeptides, or proteins. If the polynucleotide is derived from genomic DNA, expression may include splicing of the mRNA in a eukaryotic cell.

A “gene product” or alternatively a “gene expression product” refers to the amino acid (e.g., peptide or polypeptide) generated when a gene is transcribed and translated. In some embodiments, the gene product may refer to an mRNA or other RNA, such as an interfering RNA, generated when a gene is transcribed.

The term “encode” as it is applied to polynucleotides refers to a polynucleotide which is said to “encode” a polypeptide if, in its native state or when manipulated by methods well known to those skilled in the art, it can be transcribed to produce the mRNA for the polypeptide or a fragment thereof, and optionally translated to produce the polypeptide or a fragment thereof. The antisense strand is the complement of such a nucleic acid, and the encoding sequence can be deduced therefrom. Further, as used herein an amino acid sequence coding sequence refers to a nucleotide sequence encoding the amino acid sequence.

“Under transcriptional control”, which is also used herein as “directing expression of” or any grammatical variation thereof, is a term well understood in the art and indicates that transcription and optionally translation of a polynucleotide sequence, usually a DNA sequence, depends on its being operatively linked to an element which contributes to the initiation of, or promotes, transcription.

“Operatively linked” intends the polynucleotides are arranged in a manner that allows them to function in a cell.

In some embodiments, “directing the replication of” or any grammatical variation thereof is a term well understood in the art and indicates that replication of a polynucleotide sequence, usually a DNA sequence, depends on its being operatively linked to a regulatory sequence, such as an origin of replication or a primer.

The term “a regulatory sequence”, “an expression control element” or “promoter” as used herein, intends a polynucleotide that is operatively linked to a target polynucleotide to be transcribed or replicated, and facilitates the expression or replication of the target polynucleotide.

A promoter is an example of an expression control element or a regulatory sequence. Promoters can be located 5′ or upstream of a gene or other polynucleotide, that provides a control point for regulated gene transcription. Polymerase II and III are examples of promoters. In some embodiments, a regulatory sequence is bidirectional, i.e., acting as a regulatory sequence for the coding sequences on both sides of the regulatory sequence. Such bidirectional regulatory sequence may comprises, or consists essentially of, or consists of a bidirectional promoter (see for example Trinklein N D, et al. Genome Res. 2004 January; 14(1):62-6).

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting examples of promoters include the EF1alpha promoter and the CMV promoter. The EF1alpha sequence is known in the art (see, e.g., addgene.org/11154/sequences/; ncbi.nlm.nih.gov/nuccore/J04617, each last accessed on Mar. 13, 2019, and Zheng and Baum (2014) Int'l. J. Med. Sci. 11(5):404-408). The CMV promoter sequence is known in the art (see, e.g., snapgene.com/resources/plasmid-files/?set=basic_cloning_vectors&plasmid=CMV_promoter, last accessed on Mar. 13, 2019 and Zheng and Baum (2014), supra.).

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

As used herein, the term “enhancer”, as used herein, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70/a, or at least 80%, or at least 90%, or at least 95%, or at least 96%, or at least 97%, or at least 98%, or at least 99% or higher of wild-type activity, that is, activity of a full-length sequence), any truncated, mutated or otherwise modified variants of a wild-type enhancer sequence are also within the above definition.

In some embodiments, the term “vector” intends a recombinant vector that retains the ability to infect and transduce non-dividing and/or slowly-dividing cells and optionally integrate into the target cell's genome. Non-limiting examples of vectors include a plasmid, a nanoparticle, a liposome, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. In one embodiment, the viral vector is a retroviral vector.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “viral vector” is defined as a recombinantly produced virus or viral particle that comprises a polynucleotide to be delivered into a host cell, either in vivo, ex vivo or in vitro. As is known to those of skill in the art, there are 6 classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which integrates into the genomic DNA of the infected cell. The integrated DNA form is called a provirus. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827. As used herein, Multiplicity of infection (MOI) refers to the number of viral particles that are added per cell during infection.

A retrovirus such as a gammaretrovirus and/or a lentivirus comprises (a) envelope comprising lipids and glycoprotein, (b) a vector genome, which is a RNA (usually a dimer RNA comprising a cap at the 5′ end and a polyA tail at the 3′ end flanked by LTRs) derived to the target cell, (c) a capsid, and (d) proteins, such as a protease. U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. For the viral genome. and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 sequentially numbered, AAV serotypes are known in the art. Non-limiting exemplary serotypes useful in the methods disclosed herein include any of the 11 serotypes, e.g., AAV2, AAV8, AAV9, or variant or synthetic serotypes, e.g., AAV-DJ and AAV PHP.B. The AAV particle comprises, alternatively consists essentially of, or yet further consists of three major viral proteins: VP1, VP2 and VP3. In one embodiment, the AAV refers to of the serotype AAV1, AAV2, AAV4, AAV5, AAV6, AAV7, AAV8, AAV9, AAV10, AAV11, AAV12, AAV13, AAV PHP.B, or AAV rh74. These vectors are commercially available or have been described in the patent or technical literature.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

Examples of stringent hybridization conditions include: incubation temperatures of about 25° C. to about 37° C.; hybridization buffer concentrations of about 6×SSC to about 10× SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure. In some embodiments, the identity is calculated between two peptides or polynucleotides over their full-length, or over the shorter sequence of the two, or over the longer sequence of the two.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example, those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: www.ncbi.nlm.nih.gov/cgi-bin/BLAST.

In some embodiments, the polynucleotide as disclosed herein is a RNA or an analog thereof. In some embodiments, the polynucleotide as disclosed herein is a DNA or an analog thereof. In some embodiments, the polynucleotide as disclosed herein is a hybrid of DNA and RNA or an analog thereof.

In some embodiments, an equivalent to a reference nucleic acid, polynucleotide or oligonucleotide encodes the same sequence encoded by the reference. In some embodiments, an equivalent to a reference nucleic acid, polynucleotide or oligonucleotide hybridizes to the reference, a complement reference, a reverse reference, or a reverse-complement reference, optionally under conditions of high stringency.

Additionally or alternatively, an equivalent nucleic acid, polynucleotide or oligonucleotide is one having at least 70% sequence identity, or at least 75% sequence identity, or at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence, or alternatively at least 99% sequence identity to the reference nucleic acid, polynucleotide, or oligonucleotide, or alternatively an equivalent nucleic acid hybridizes under conditions of high stringency to a reference polynucleotide or its complementary. In one aspect, the equivalent must encode the same protein or a functional equivalent of the protein that optionally can be identified through one or more assays described herein. In addition or alternatively, the equivalent of a polynucleotide would encode a protein or polypeptide of the same or similar function as the reference or parent polynucleotide.

The term “transduce” or “transduction” refers to the process whereby a foreign nucleotide sequence is introduced into a cell. In some embodiments, this transduction is done via a vector, viral or non-viral.

The term “protein”, “peptide” and “polypeptide” are used interchangeably and in their broadest sense to refer to a compound of two or more subunit amino acids, amino acid analogs or peptidomimetics. The subunits (which are also referred to as residues) may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, an amino acid (aa) or nucleotide (nt) residue position in a sequence of interest “corresponding to” an identified position in a reference sequence refers to that the residue position is aligned to the identified position in a sequence alignment between the sequence of interest and the reference sequence. Various programs are available for performing such sequence alignments, such as Clustal Omega and BLAST.

Non-limiting suitable alignment programs include Clustal Omega (accessible at www.ebi.ac.uk/Tools/msa/clustalo/), Needle (EMBOSS, accessible at /www.ebi.ac.uk/Tools/psa/emboss_needle/), Stretcher (EMBOSS, accessible at www.ebi.ac.uk/Tools/psa/emboss_stretcher/), Water (EMBOSS, accessible at www.ebi.ac.uk/Tools/psa/emboss_water), Matcher (EMBOSS, accessible at www.ebi.ac.uk/Tools/psa/emboss_matcher), LALIGN (accessible at www.ebi.ac.uk/Tools/psa/lalign) and BLAST (accessible at blast.ncbi.nlm.nih.gov/Blast.cgi?PAGE_TYPE=BlastSearch&PROG_DEF=blastn&BLAST_PROG_DEF=blastn&BLAST_SPEC=GlobalAln&LINK_LOC=BlastHomeLink and www.ncbi.nlm.nih.gov/tools/cobalt/cobalt.cgi?LINK_LOC=BlastHomeLink). In aspects of this disclosure, the Clustal Omega alignment program is used to determine sequence identity.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits, llama and mice, as well as non-mammalian species, such as shark immunoglobulins.

Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, murine or humanized non-primate antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7th Ed., W.H. Freeman & Co., 2013; Murphy, Janeway's Immunobiology, 8th Ed., Garland Science, 2014; Male et al., Immunology (Roitt), 8th Ed., Saunders, 2012; Parham, The Immune System, 4th Ed., Garland Science, 2014. In some embodiments, the term “antibody” refers to a single-chain variable fragment (scFv, or ScFV). In some embodiments, the term “antibody” refers to more than one single-chain variable fragments (scFv, or ScFV) linked with each other, optionally via a peptide linker or another suitable component as disclosed herein. In some embodiments, an antibody is a monoclonal antibody. In some embodiments, an antibody is a monospecific antibody or a multispecific antibody, such as a bispecific antibody or a trispecific antibody. The species of the antibody can be a human or non-human, e.g., mammalian.

In certain embodiments, an antigen binding fragment of an antibody contains at least one variable domain optionally covalently linked to at least one constant domain. Non-limiting, exemplary configurations of variable and constant domains that are found within an antigen-binding fragment of an antibody of the present invention include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains are either directly linked to one another or are linked by a full or partial hinge or linker region. A hinge region can consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen binding fragment of an antibody of the present disclosure can comprise a homo-dimer or hetero-dimer (or other multimer) of any of the variable and constant domain configurations listed herein in non-covalent association with one another and/or with one or more monomeric VH or VL domain (e.g., by disulfide bond(s)).

As used herein, an epitope refers to contiguous or non-contiguous amino acid residues in an antigen, such as those adjacent to each other in a three-dimensional structure of the antigen, wherein those residues are recognized and bound by an antibody or another component of the immune system.

As used herein, the term “multispecific” refers to capability of binding to more than one epitopes or antigens which are different from each other. In some embodiments, the term “multispecific” refers to comprising, or consisting essentially of, or consisting of more than one antigen binding sequences or antigen ligands, optionally linked together by a peptide linker or another component as disclosed herein. In further embodiments, the term “multispecific” refers to comprising, or consisting essentially of, or consisting of more than one antigen binding sequences (such as scFv), optionally linked together by a peptide linker or another component as disclosed herein. In some embodiments, the more than one (such as two) epitopes are located in the same antigen. Alternatively, the more than one (such as two) epitopes are from at least two antigens. In some embodiments, the ligand refers a ligand of the antigen. In some embodiments, a multispecific antibody comprises, or consists essentially of, or consists of at least two antigen binding sequences. In some embodiments, a multispecific antibody comprises, or consists essentially of, or consists of at least one antigen binding sequence and at least one ligand (such as a polypeptide comprising or consisting of a binding domain of the antigen's receptor).

Accordingly, a bispecific antibody (abbreviated as BsAb) refers to an antibody capable of binding to two epitopes or antigens which are different from each other. In some embodiments, a bispecific antibody comprises, or consists essentially of, or consists of two antigen binding sequences or antigen ligands, optionally linked together by a peptide linker or another component as disclosed herein. In further embodiments, a bispecific antibody comprises, or consists essentially of, or consists of two antigen binding sequences (such as scFv), optionally linked together by a peptide linker or another component as disclosed herein. In some embodiments, a bispecific antibody comprises, or consists essentially of, or consists of one antigen binding sequence recognizing and binding the first epitope and one ligand recognizing and binding the antigen comprising the second epitope. In some embodiments, the two epitopes are located in the same antigen. Alternatively, the two epitopes are from two antigens which are different from each other. In some embodiments, the ligand refers to a ligand of the antigen, such as a polypeptide comprising or consisting of a binding domain of the antigen's receptor. In some embodiments, a bispecific antibody comprises, or consists essentially of, or consists of at least two antigen binding sequences. In some embodiments, a bispecific antibody comprises, or consists essentially of, or consists of at least one antigen binding sequence and at least one ligand.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

In terms of antibody structure, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains,” such as constant domain and variable domain, respectively). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located (heavy chain regions labeled CDRH or HCDR and light chain regions labeled CDRL or LCDR). Thus, a HCDR3 is the CDR3 from the variable domain of the heavy chain of the antibody in which it is found, whereas a LCDR1 is the CDR1 from the variable domain of the light chain of the antibody in which it is found.

As used herein, a single-chain variable fragment (scFv or ScFV), also referred to herein as a fragment or an antigen binding fragment of an antibody, and is a fusion protein of the variable regions of the heavy (V_{H}) and light chains (V_{L}) of immunoglobulins, optionally connected with a short linker peptide of about 10 to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker.

As used herein, a fragment crystallizable (Fc) region refers to the tail region of an antibody that stabilizes the antibody, such as a bispecific antibody, and optionally interacts with (such as binds) an Fc receptor on an immune cell or on a platelet or that binds a complement protein.

The polypeptide or an equivalent thereof, can be followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus (C-terminus). Additionally or alternatively, the polypeptide or an equivalent thereof can further comprises an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the amine-terminus (N-terminus).

An equivalent of a reference polypeptide comprises, consists essentially of, or alternatively consists of an polypeptide having at least 80%, or at least 85%, or at least 90%, or at least 95%, or at least about 96%, or at least 97%, or at least 98%, or at least 99% amino acid identity to the reference polypeptide (as determined, in one aspect using the Clustal Omega alignment program), such as the antibody or antigen binding fragment thereof as disclosed herein, or a polypeptide that is encoded by a polynucleotide that hybridizes under conditions of high stringency to the complementary sequence of a polynucleotide encoding the reference polypeptide, such as an antibody or antigen binding fragment thereof as disclosed herein, optionally wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

Alternative embodiments include one or more of the CDRs (e.g., CDR1, CDR2, CDR3) from the LC variable region substituted with appropriate CDRs from other antibody CDRs, or an equivalent of each thereof. Accordingly, and as an example, the CDR1 and CDR2 from the LC variable region can be combined with the CDR3 of another antibody's LC variable region, and in some aspects, can include an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the term “equivalent” or “biological equivalent” of an antibody means the ability of the antibody to selectively bind its epitope protein or a fragment thereof as measured by ELISA or other suitable methods is substantively maintained, for example, at a level of at least 50%, or at least 55%, or at least 60%, or at least 65%, or at least 70%, or at least 75%, or at least 80%, or at least 85%, or at least 90%, or at least 95%, or at least 99%, or more. Biologically equivalent antibodies include, but are not limited to, those antibodies, peptides, antibody fragments, antibody variant, antibody derivative and antibody mimetics that bind to the same epitope as the reference antibody. Additionally or alternatively, the equivalent and the reference antibody shares the same set of CDRs but other amino acids are modified.

In some embodiments, a first sequence (nucleic acid sequence or amino acid) is compared to a second sequence, and the identity percentage or edit distance between the two sequences can be calculated. In further embodiments, the first sequence can be referred to herein as an equivalent and the second sequence can be referred to herein as a reference sequence. In yet further embodiments, the identity percentage is calculated based on the full-length sequence of the first sequence. In other embodiments, the identity percentage is calculated based on the full-length sequence of the second sequence.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity, or at least about 85% homology or identity, or alternatively at least about 90% homology or identity, or alternatively at least about 95% homology or identity, or alternatively 98% or 99% homology or identity (in one aspect, as determined using the Clustal Omega alignment program) and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complementary sequence.

In some embodiments, an antibody as disclosed herein comprises, or consists essentially of, or yet further consists of an anybody variant. The term “antibody variant” intends to include antibodies produced in a species other than a mouse. It also includes antibodies containing post-translational modifications to the linear polypeptide sequence of the antibody or a fragment thereof. It further encompasses fully human antibodies.

In some embodiments, an antibody as disclosed herein comprises, or consists essentially of, or yet further consists of an antibody derivative. The term “antibody derivative” is intended to encompass molecules that bind an epitope as defined above and which are modifications or derivatives of a native monoclonal antibody of this disclosure. Derivatives include, but are not limited to, for example, bispecific, multispecific, heterospecific, trispecific, tetraspecific, multispecific antibodies, diabodies, chimeric, recombinant and humanized.

As used herein, the term “specific binding” or “binding” means the contact between an antibody and an antigen with a binding affinity of at least 10⁻⁶ M. In certain embodiments, antibodies bind with affinities of at least about 10⁻⁷ M, and preferably at least about 10⁻⁸ M, at least about 10⁻⁹ M, at least about 10⁻¹⁰ M, at least about 10⁻¹¹ M, or at least about 10⁻¹² M.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens. In some embodiments, the antigen as referred to herein is a SARS-CoV-2 or a pseudovirus thereof. In some embodiments, the antigen as referred to herein is an N protein of a SARS-CoV-2, or a fragment thereof. In further embodiments, the fragment is an immunogenic fragment. In yet further embodiments, the antigen comprises, or consists essentially of, or yet further consists of SEQ ID NO: 1.

In some embodiments, antigen of a binding moiety, such as an antibody, an antigen binding fragment thereof, can be provided herein in a format of “antigen” followed by the binding moiety (such as an anti-S antibody), or having “anti” or “anti-” before the antigen and the binding moiety after the antigen (such as an anti-N antibody), or the binding moiety followed by “to” or “directed to” and then the antigen (such as an antibody to N protein).

In some embodiments, a fragment of a protein can be an immunogenic fragment. As used herein, the term “immunogenic fragment” refers to such a polypeptide fragment, which at least partially retains the immunogenicity of the protein from which it is derived. In some embodiments, the immunogenic fragment is at least about 3 amino acid (aa) long, or at least about 4 aa long, or at least about 5 aa long, or at least about 6 aa long, or at least about 7 aa long, or at least about 8 aa long, or at least about 9 aa long, or at least about 10, aa long, or at least about 15, aa long, or at least about 20 aa long, or at least about 25 aa long, or at least about 30 aa long, or at least about 35 aa long, or at least about 40 aa long, or at least about 50 aa long, or at least about 60 aa long, or at least about 70 aa long, or at least about 80 aa long, or at least about 90 aa long, or at least about 100 aa long, or at least about 120 aa long, or at least about 150 aa long, or at least about 200, or longer.

As used herein, the terms “antigen binding fragment,” “fragment,” and “antibody fragment” are used interchangeably to refer to any fragment that comprises a portion of a full-length antibody, generally at least the antigen binding portion or the variable region thereof. Examples of antibody fragments include, but are not limited to, diabodies, single-chain antibody molecules, multi-specific antibodies, Fab, Fab′, F(ab′)₂, Fv or scFv. In some embodiments, the term “antigen binding domain” refers to any protein or polypeptide domain that can specifically bind to an antigen target.

The term “culturing” refers to the in vitro or ex vivo propagation of cells or organisms on or in media of various kinds. It is understood that the descendants of a cell grown in culture may not be completely identical (i.e., morphologically, genetically, or phenotypically) to the parent cell.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, canine, bovine, porcine, murine, rat, avian, reptilian and human. These cells are commercially available, for example, from the American Type Culture Collection (ATCC).

“Prokaryotic cells” that usually lack a nucleus or any other membrane-bound organelles and are divided into two domains, bacteria and archaea. Additionally, instead of having chromosomal DNA, these cells' genetic information is in a circular loop called a plasmid. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to bacillus bacteria, E. coli bacterium, and Salmonella bacterium. These cells are commercially available, for example, from the American Type Culture Collection (ATCC).

As used herein, a “hybridoma” refers to the product of a cell-fusion between a cultured neoplastic lymphocyte and a primed B- or T-lymphocyte which expresses the specific immune potential of the parent cell, such as an antibody.

In one embodiment, the term “disease” or “disorder” as used herein refers to a SARS-CoV-2 infection, being exposed to SARS-CoV-2 viruses, a status of being diagnosed with a SARS-CoV-2 infection, a status of being suspect of having a SARS-CoV-2 infection (such as having a symptom of a SARS-CoV-2 infection), or a status of at high risk of having a SARS-CoV-2 infection (such as being suspect of being exposed to SARS-CoV-2 viruses). In one embodiment, the term “disease” or “disorder” as used herein refers to COVID-19, a status of being diagnosed with COVID-19, a status of being suspect of having COVID-19, or a status of at high risk of having COVID-19. In one embodiment, the term “disease” or “disorder” as used herein refers to moderate severity COVID-19, a status of being diagnosed with moderate severity COVID-19, a status of being suspect of having moderate severity COVID-19, or a status of at high risk of having moderate severity COVID-19. Additionally or alternatively, the term “disease” or “disorder” as used herein refers to acute respiratory distress syndrome, a status of being diagnosed with acute respiratory distress syndrome, a status of being suspect of having acute respiratory distress syndrome, or a status of at high risk of having acute respiratory distress syndrome. In further embodiments, the disease or disorder is directly or indirectly caused by a SARS-CoV-2 infection. In some embodiments, the composition or methods as disclosed herein is suitable for treating a disease.

As used herein, “moderate severity COVID-19” refers to individuals who have evidence of lower respiratory disease by clinical assessment or imaging and a saturation of oxygen (SpO₂) ≥94% on room air at sea level. While the diagnosis can be made on clinical grounds; chest imaging (radiograph, CT scan, ultrasound) may assist in diagnosis and identify or exclude pulmonary complications.

As used herein, “acute respiratory distress syndrome” or “ARDS” is a type of respiratory failure characterized by rapid onset of widespread inflammation in the lungs. Symptoms include shortness of breath, rapid breathing, and bluish skin coloration.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a disease.

In some embodiments, a subject as referred to herein has been treated with a standard care for the disease. In some embodiments, a subject as referred to herein is concurrently treated with a standard care of the disease. In some embodiments, a subject as referred to herein will be treated with a standard care of the disease. As used herein, “standard of care” or “SOC” refers to the diagnostic and treatment process that a clinician should follow for a certain type of patient, illness, or clinical circumstance. SOC may include administration of drugs that are being used in clinical practice for the treatment of COVID-19 (e.g. lopinavir/ritonavir; darunavir/cobicistat; hydroxy/chloroquine, tocilizumab, etc.), other than those used as part of another clinical trial.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

In some embodiments, the terms “treating,” “treatment,” and the like, as used herein, mean ameliorating a disease, so as to reduce, ameliorate, or eliminate its cause, its progression, its severity, or one or more of its symptoms, or otherwise beneficially alter the disease in a subject. Reference to “treating,” or “treatment” of a patient is intended to include prophylaxis. Treatment may also be preemptive in nature, i.e., it may include prevention of disease in a subject exposed to or at risk for the disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “passive immunity” refers to the transfer of immunity from one subject to another through the transfer of antibodies. Passive immunity may occur naturally, as when maternal antibodies are transferred to a fetus. Passive immunity may also occur artificially as when antibody compositions are administered to non-immune subjects. Antibody donors and recipients may be human or non-human subjects. Antibodies may be polyclonal or monoclonal, may be generated in vitro or in vivo, and may be purified, partially purified, or unpurified depending on the embodiment. In some embodiments described herein, passive immunity is conferred on a subject in need thereof through the administration of antibodies or antigen-binding fragments that specifically recognize or bind to a particular antigen, such as an N protein. In some embodiments, passive immunity is conferred through the administration of an isolated or recombinant polynucleotide encoding an antibody or antigen-binding fragment that specifically recognizes or binds to a particular antigen, such as an N protein.

“Immune response” broadly refers to the antigen-specific responses of lymphocytes to foreign substances. The terms “immunogen” and “immunogenic” refer to molecules with the capacity to elicit an immune response. All immunogens are antigens, however, not all antigens are immunogenic. An immune response disclosed herein can be humoral (via antibody activity) or cell-mediated (via T cell activation). The response may occur in vivo or in vitro. The skilled artisan will understand that a variety of macromolecules, including proteins, nucleic acids, fatty acids, lipids, lipopolysaccharides and polysaccharides have the potential to be immunogenic. The skilled artisan will further understand that nucleic acids encoding a molecule capable of eliciting an immune response necessarily encode an immunogen. The artisan will further understand that immunogens are not limited to full-length molecules, but may include partial molecules.

As used herein, the term “neutralization” refers to a process of neutralizing a pathogen by antibody acting of a receptor or an antigen of the pathogen. In some embodiments, the term “neutralization” refers to the process by which antibody alone or antibody plus complement neutralizes the infectivity of a virus, such as a SARS-CoV-2. Accordingly, a “neutralizing antibody” or “nAb” refers to an antibody that blocks viral infection of a cell.

In some embodiments, an antibody or antigen binding fragment thereof as disclosed herein mediates an ADCC in a subject in need thereof, and treating the subject. “Antibody-dependent cell-mediated cytotoxicity” or “ADCC” refers to a form of cytotoxicity in which secreted antibodies bound onto Fc receptors (FcRs) present on certain cytotoxic cells (for example NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, for example, in an animal model such as that disclosed in Clynes et al. Proc. Natl. Acad. Sci. (USA) 95:652-656 (1998). Additional polypeptide variants with altered Fc region amino acid sequences (polypeptides with a variant Fc region) and increased or decreased ADCC activity are described, for example, in U.S. Pat. Nos. 7,923,538, and 7,994,290.

“Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, antibody or composition described herein.

As used herein, the term “label” or a detectable label intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected, or it may be quantified. A response that is simply detected generally comprises a response whose existence merely is confirmed, whereas a response that is quantified generally comprises a response having a quantifiable (e.g., numerically reportable) value such as an intensity, polarization, or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In some embodiments, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, a purification label or maker refers to a label that may be used in purifying the molecule or component that the label is conjugated to, such as an epitope tag (including but not limited to a Myc tag, a human influenza hemagglutinin (HA) tag, a FLAG tag), an affinity tag (including but not limited to a glutathione-S transferase (GST), a poly-Histidine (His) tag, Calmodulin Binding Protein (CBP), or Maltose-binding protein (MBP)), or a fluorescent tag.

In some embodiments, a detectable marker can be used to produce a detectable signal upon binding of two moieties, such as an antibody and its antigen. In some embodiments, one of the two moieties is immobilized, the mobilized moiety is the provided for binding, and unbound mobilized moiety is removed by washing with a suitable solution. Accordingly, any detectable marker can be directly or indirectly conjugated to the mobilized moiety, and the detectable signal obtained after the washing step indicates binding between the two moieties. In other embodiments, a detectable signal can be generated if two moieties are in the proximity with each other. For example, one part of a detectable marker, such as a fluorescent protein, can be directly or indirectly conjugated to the first moiety while the other part of the detectable marker is directly or indirectly conjugated to the second moiety, and two parts of the detectable markers, when in the proximity with each other, generate a detectable signal. Alternatively, fluorescence resonance energy transfer (FRET) can also be used.

As used herein, the term “ELISA” refers to enzyme-linked immunosorbant assay. Numerous methods and applications for carrying out an ELISA are well known in the art, and provided in many sources (See, e.g., Crowther, “Enzyme-Linked immunosorbant Assay (ELISA),” in Molecular Biomethods Handbook, Rapley et al. (eds.], pp. 595-617, Humana Press, Inc., Totowa, N.J., 1998). In some embodiments, an ELISA is a “direct ELISA”, where a target-binding molecule, such as a cell, cell lysate, or isolated protein, is first bound and immobilized to a microtiter plate well. In an alternative embodiment, an ELISA is a “sandwich ELISA”, where a target-binding molecule is attached to the substrate by capturing it with an antibody that has been previously bound to the microtiter plate well. The ELISA method detects an immobilized ligand-receptor complex (binding) by use of fluorescent detection of fluorescently labeled ligands or an antibody-enzyme conjugate, where the antibody is specific for the antigen of interest, such as a phage virion, while the enzyme portion allows visualization and quantitation by the generation of a colored or fluorescent reaction product. The conjugated enzymes commonly used in the ELISA include horseradish peroxidase, urease, alkaline phosphatase, glucoamylase or O-galactosidase. The intensity of color development is proportional to the amount of antigen present in the reaction well.

A lateral flow immunoassay refers to an assay format in which a sample is applied to a lateral flow matrix. The sample flows along the lateral flow matrix, and one or more analyte components to be detected in the sample react with at least one reagent which is provided in or added to the lateral flow matrix. At least one reagent is typically immobilized in the device for reaction with the analyte component to be detected or a reagent thereof, and labels are typically employed to measure the extent of reaction with an immobilized reagent. See, e.g., U.S. patents and patent application publications: U.S. Pat. Nos. 5,602,040; 5,622,871; 5,656,503; 6,187,598; 6,228,660; 6,818,455; 2001/0008774; 2005/0244986; 6,352,862; 2003/0207465; 2003/0143755; 2003/0219908; U.S. Pat. Nos. 5,714,389; 5,989,921; 6,485,982; 11/035,047; 5,656,448; 5,559,041; 5,252,496; 5,728,587; 6,027,943; 6,506,612; 6,541,277; 6,737,277 B1; U.S. Pat. Nos. 5,073,484; 5,654,162; 6,020,147; 4,956,302; 5,120,643; 6,534,320; 4,942,522; 4,703,017; 4,743,560; 5,591,645; and RE 38,430.

As used herein, a biological sample, or a sample, is obtained from a subject. Exemplary samples include, but are not limited to, cell sample, tissue sample, biopsy, liquid samples such as blood and other liquid samples of biological origin, including, but not limited to, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood.

In some embodiments, the sample may be an upper respiratory specimen, such as a nasopharyngeal (NP) specimen, an oropharyngeal (OP) specimen, a nasal mid-turbinate swab, an anterior nares (nasal swab) specimen, or nasopharyngeal wash/aspirate or nasal wash/aspirate (NW) specimen.

In some embodiments, the samples include fluid from a subject, including, without limitation, blood or a blood product (e.g., serum, plasma, or the like), umbilical cord blood, amniotic fluid, cerebrospinal fluid, spinal fluid, lavage fluid (e.g., bronchoalveolar, gastric, peritoneal, ductal, ear, arthroscopic), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a liquid biological sample is a blood plasma or serum sample. The term “blood” as used herein refers to a blood sample or preparation from a subject. The term encompasses whole blood, blood product or any fraction of blood, such as serum, plasma, buffy coat, or the like as conventionally defined. In some embodiments, the term “blood” refers to peripheral blood. Blood plasma refers to the fraction of whole blood resulting from centrifugation of blood treated with anticoagulants. Blood serum refers to the watery portion of fluid remaining after a blood sample has coagulated. Fluid samples often are collected in accordance with standard protocols hospitals or clinics generally follow. For blood, an appropriate amount of peripheral blood (e.g., between 3-40 milliliters) often is collected and can be stored according to standard procedures prior to or after preparation.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” or “delivery” of a cell or vector or other agent and compositions containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

The term administration shall include without limitation, administration by oral, parenteral (e.g., intramuscular, intraperitoneal, intravenous, intracerebroventricular (ICV), intrathecal, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “excipient” refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

A combination as used herein intends that the individual active ingredients of the compositions are separately formulated for use in combination, and can be separately packaged with or without specific dosages. The active ingredients of the combination can be administered concurrently or sequentially.

In some embodiments, an antibody or antigen binding fragment thereof is administered in an effective amount. An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific agent employed, bioavailability of the agent, the route of administration, the age of the animal and its body weight, general health, sex, the diet of the animal, the time of administration, the rate of excretion, the drug combination, and the severity of the particular disorder being treated and form of administration. In general, one will desire to administer an amount of the agent that is effective to achieve a serum level commensurate with the concentrations found to be effective in vivo. These considerations, as well as effective formulations and administration procedures are well known in the art and are described in standard textbooks.

“Therapeutically effective amount” of an agent refers to an amount of the agent that is an amount sufficient to obtain a pharmacological response; or alternatively, is an amount of the agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

The SARS-CoV-2 genome is composed of approximately 30,000 nucleotides, which encodes four structural proteins include spike (S) protein, envelope (E) protein, membrane (M) protein, and nucleocapsid (N) protein (Lu et al., 2020, The Lancet 395, 565-574). Among them, sera from convalescent SARS patients cross-neutralized SARS-2-S-driven entry (Hoffmann et al., 2020, Cell. 2020 Apr. 16; 181(2):271-280.e8); and N protein is a highly immunogenic and abundantly expressed protein during infection (Shang et al., 2005, Biochem. Biophys. Res. Commun. 336, 110-117; and Liu et al., 2006, Vaccine 24, 3100-3108).

Furthermore, N protein is frequently used in vaccine development and serological assays (Ahmed et al., 2020, Preliminary Identification of Potential Vaccine Targets for the COVID-19 Coronavirus (SARS-CoV-2) Based on SARS-CoV Immunological Studies. Viruses 12). Serologically detected specific antibodies against the N protein in the serum of SARS patients have higher sensitivity and longer persistence than those antibodies to other structural proteins of SARS-CoV (Tan et al., 2004, Clin. Diagn. Lab. Immunol. 11, 362-371; and Shi et al., 2003, J. Clin. Microbiol. 41, 5781-5782). Moreover, anti-N antibodies have been detected with high specificity in the early stage of infection (Leung et al., 200, J. Infect. Dis. 190, 379-3864).

As used herein the terms “Nucleoprotein protein,” “Nucleoprotein,” “N-protein” and “N protein” are used interchangeably to refer to a viral protein that is structurally associated with the viral DNA or RNA. In some embodiments, an N protein as used herein refers to a SARS-CoV-2 protein that packages the positive strand viral genome RNA into a helical ribonucleocapsid (RNP) and plays a fundamental role during virion assembly through its interactions with the viral genome and membrane protein M. Non-limiting exemplary sequences of this protein or the underlying gene may be found under UniProtKB: P0DTC9, which is incorporated by reference herein in its entirety. In some embodiments, an N protein as used herein comprises, or consists essentially of, or yet further consists of a polypeptide as set forth in SEQ ID NO: 1 or a natural variant thereof. In some embodiments, the natural variant comprises, or consists essentially of, or yet further consists of SEQ ID NO: 1 comprising leucine (L) as its third amino acid residue instead of aspartic acid (D). Additionally or alternatively, the natural variant comprises, or consists essentially of, or yet further consists of SEQ ID NO: 1 comprising phenylalanine (F) as its 235^{th} amino acid instead of serine (S).

### Modes for Carrying Out the Disclosure

The disclosure provides antibodies and antigen binding fragments thereof that bind to SARS-CoV-2 protein, e.g., the SARS-CoV2 N protein. In one embodiment, the disclosure provides an isolated antibody or antigen binding fragment thereof that binds to SEQ ID NO:1. This disclosure also provides compositions for use of the antibodies and fragments thereof, and compositions for manufacturing the same.

The provided antibodies can be used to diagnose, treat, or monitor infection by SARS-CoV-2 virus. In some embodiments, the antibodies or fragments thereof as described herein can be used for various in vitro molecular biology applications such as, enzyme-linked immunosorbent assays (ELISA), Western blots, immunohistochemistry, immunocytochemistry, flow cytometry and fluorescence-activated cell sorting (FACS), immunoprecipitation, or enzyme-linked immunospot assays. In some embodiments, the antibodies or fragments thereof can be packaged in kits with or without additional reagents known to those of skill in the art for practicing any of the molecular biology techniques as disclosed herein.

In another aspect, the present disclosure provides a method of preventing or treating a disease, such as a SARS-CoV-2 infection, in a subject in need thereof, comprising, or consisting essentially of, or yet further consisting of administering to the subject, optionally a therapeutically or prophylactically effective amount of, a pharmaceutical composition comprising, or consisting essentially of, or yet further consisting of one or more of the antibodies or antigen binding fragments as described herein. Such a method can comprise, or consists essentially of, or yet further consists of administration of any dose of the antibodies described herein effective for ameliorating or treating symptoms of SARS-CoV-2 infection. Methods for determining if the disease has been treated or prevented are known in the art and include a reduction in symptoms, or severity of symptoms or the presence of neutralizing antibodies in the subject being treated.

### Antibodies

The disclosure provides antibodies and antigen binding fragments thereof that bind specifically to SEQ ID NO:1. The term “antibody” as used herein, includes both full-length immunoglobulins and antibody fragments that bind to the same antigens. The antibodies can be, e.g., a monoclonal, polyclonal, chimeric, humanized, or single chain antibody. In one aspect, the antibodies and fragments thereof are described by the CDRs. In another aspect they are described by the heavy and light chains. Polynucleotides encoding such antibodies and fragments thereof are further provided herein.

The disclosure provides the antibodies SCT-Pa01, SCT-Pa02, SCT-Pa03, SCT-Pa04, SCT-Pa05, SCT-Pa06, SCT-Pa07, SCT-Pa08, SCT-Pa09, SCT-Pa10, SCT-Pa11, SCT-Pa12, SCT-Pa13, SCT-Pa14, SCT-Pa15, SCT-Pa16, SCT-Pa17, SCT-Pa18, SCT-Pa19, and SCT-Pa20. Each of these is a murine monoclonal antibody. Tables 2-3 provide additional information relates to the designated antibodies. It would also be understood by one of skill in the art that the disclosure herein numbered “SCT-Pa” followed by a two-digit number describes the antibody with such designation as well as its equivalents.

Additionally, recombinant anti-SARS-CoV-2 N protein antibodies, such as chimeric and humanized monoclonal antibodies, comprising both human and non-human portions, which can be made using standard recombinant DNA techniques, are within the scope of the disclosure. Such chimeric and humanized monoclonal antibodies can be produced by recombinant DNA techniques such as, for example, the methods described in U.S. Pat. No. 7,112,421; Better et al. (1988) Science 240:1041-1043; or Liu et al. (1987) Proc. Natl. Acad. Sci. USA 84:3439-3443.

In one aspect, provided is an antibody or an antigen binding fragment thereof. The antibody or antigen binding fragment thereof comprises, or consists essentially of, or yet further consists of one or more CDRs as listed in Table 2. In some embodiments, the antibody or antigen binding fragment thereof comprises, or consists essentially of, or yet further consists of an HCDR1, an HCDR2 and an HCDR3 as listed in Table 2. In further embodiments, the HCDR1, HCDR2 and HCDR3 are listed in the same row in Table 2. Additionally or alternatively, the antibody or antigen binding fragment thereof comprises, or consists essentially of, or yet further consists of an LCDR1, an LCDR2, and an LCDR3 as listed in Table 2. In further embodiments, the LCDR1, LCDR2 and LCDR3 are listed in the same row in Table 2. In some embodiments, the antibody or antigen binding fragment thereof comprises, or consists essentially of, or yet further consists of an HCDR1, an HCDR2, an HCDR3, an LCDR1, an LCDR2, and an LCDR3 as listed in Table 2. In further embodiments, the HCDR1, HCDR2, HCDR3, LCDR1, LCDR2 and LCDR3 are listed in the same row in Table 2. Polynucleotides encoding such CDRs and combinations thereof are further provided herein.

TABLE 2
(SEQ ID Nos for each listed sequence are provided in parenthesis)
CDR sequences of mAbs generated against SARS-CoV-2 N protein
(SEQ ID Nos for each listed sequence are provided in parenthesis)

| mAb AA ID | HCDR1 | HCDR2 | HCDR3 | LCDR1 | LCDR2 | LCDR3 |
|---|---|---|---|---|---|---|
| SCT-Pa01 | GFSLSTFGMG | IWWDDD | TRMREDSSDYVYYIMDY | SSVSY | RTS | QLYHSYPPTWT |
| | (44) | (45) | (46) | (100) | (101) | (102) |
| SCT-Pa02 | GFSLTSYG | IWSGGG | ARGAYYSLDY | ESVDYDGDNY | ATS | LQSYDVPYT |
| | (47) | (48) | (49) | (103) | (104) | (105) |
| SCT-Pa03 | GYTFTDYY | IYPGNNY | AKDDGYRFAY | DHINNW | GAT | QQYWSTPPT |
| | (50) | (51) | (52) | (106) | (107) | (108) |
| SCT-Pa04 | GYSFTSYV | INPYNDG | SRDDYSNPPFAY | QSVSNN | YAS | QQDYDSPYT |
| | (53) | (54) | (55) | (109) | (110) | (111) |
| SCT-Pa05 | GFNIKDYY | IDPEDGE | ARSLYY | QSLLNSGDQKNY | ETS | LNDLTYPLT |
| | (56) | (57) | (58) | (112) | (113) | (114) |
| SCT-Pa06 | GFSLSTYGMG | IWWNDD | AQIPAYYSNSWFAY | SSVSSGN | RTS | QQWSGYPYT |
| | (59) | (60) | (61) | (115) | (101) | (116) |
| SCT-Pa07 | GYTFTDYY | IYTNNGD | ASLYYSNFFDS | SNVKY | YTS | QQFTSSPFT |
| | (50) | (62) | (63) | (117) | (118) | (119) |
| SCT-Pa08 | GYTFTEYS | FNPGTGS | ARHGGRWVIQWNFDV | QNLLYSSNQKNF | WAS | QQYYRYPFT |
| | (64) | (65) | (66) | (120) | (121) | (122) |
| SCT-Pa09 | GYTFTSSW | ISLTNGD | TIIPDGYSLYWYFDV | SSVNY | ATS | QQWSSNPPT |
| | (67) | (68) | (69) | (123) | (104) | (124) |
| SCT-Pa10 | GYAFSTSW | IYPGDGE | ARGANGDYLFYLDY | QDISDE | YTS | QQYSKLPYT |
| | (70) | (71) | (72) | (125) | (118) | (126) |
| SCT-Pa11 | GYTFTSYV | INPYNDG | SRDDYNNRFAY | ENIYSY | NAK | LHHFGTPWT |
| | (73) | (54) | (74) | (127) | (128) | (129) |
| SCT-Pa12 | GYSFTSYG | IYPRSGN | ARSGDGYLYAMDY | QDISNY | YTS | QQYSKLPYT |
| | (75) | (76) | (77) | (130) | (118) | (126) |
| SCT-Pa13 | GFSLSSYA | IWSGGT | ARGAYYSLDH | QSLANSYGNTY | GIS | LOGTHQYT |
| | (78) | (79) | (80) | (131) | (132) | (133) |
| SCT-Pa14 | GFNIKDDY | IDPANGN | FYSFYIPYTMDF | SNVKY | YTS | QQFTSSPYT |
| | (81) | (82) | (83) | (117) | (118) | (134) |
| SCT-Pa15 | GFSSTSYA | IWSGGT | ARGNYYGMDY | ESIDYDGDSY | AAS | LOSYETPWT |
| | (84) | (79) | (85) | (135) | (136) | (137) |
| SCT-Pa16 | GYTFTSYW | VNPSNGG | ARRNGFPYYFDY | QNVGTN | SAS | HQYNSSPLT |
| | (86) | (87) | (88) | (138) | (139) | (140) |
| SCT-Pa17 | GYAFTSSW | VYPRSSS | ARGYGSYWYFDV | EDIYNR | GAT | QEYWSLSPT |
| | (89) | (90) | (91) | (141) | (107) | (142) |
| SCT-Pa18 | GYTFTSYW | IAPSDSY | AGGDYYGYDVKTWFAY | QDISNY | YTS | QQYSKLPYT |
| | (86) | (92) | (93) | (130) | (118) | (126) |
| SCT-Pa19 | GYTFTNYW | IDPSDSE | ARDSYGWYFDV | QNVGSN | SAS | QQYNNYPLT |
| | (94) | (95) | (96) | (143) | (139) | (144) |
| SCT-Pa20 | GYTFTDYW | IYPGSSD | ARRPLFHAMDY | QSINYNGDNY | TAS | QQSKEFPWT |
| | (97) | (98) | (99) | (145) | (146) | (147) |

In some embodiments, the antibody or antigen binding fragment thereof comprises, or consists essentially of, or yet further consists of:
(SCT-Pa01) a heavy chain (HC) complementarity determining region (CDR) 1 (HCDR1) that comprises, or consists essentially of, or yet further consists of the amino acid sequence of SEQ ID NO: 44, an HC CDR 2 (HCDR2) that comprises, or consists essentially of, or yet further consists of the amino acid sequence of SEQ ID NO: 45, and an HC CDR 3 (HCDR3) that comprises, or consists essentially of, or yet further consists of the amino acid sequence of SEQ ID NO: 46;
(SCT-Pa02) an HCDR1 that comprises, or consists essentially of, or yet further consists of the amino acid sequence of SEQ ID NO: 47, an HCDR2 that comprises, or consists essentially of, or yet further consists of the amino acid sequence of SEQ ID NO: 48, and an HCDR3 that comprises, or consists essentially of, or yet further consists of the amino acid sequence of SEQ ID NO: 49;
(SCT-Pa03) an HCDR1 that comprises, or consists essentially of, or yet further consists of the amino acid sequence of SEQ ID NO: 50, an HCDR2 that comprises, or consists essentially of, or yet further consists of the amino acid sequence of SEQ ID NO: 51, and an HCDR3 that comprises, or consists essentially of, or yet further consists of the amino acid sequence of SEQ ID NO: 52;
(SCT-Pa04) an HCDR1 that comprises, or consists essentially of, or yet further consists of the amino acid sequence of SEQ ID NO: 53, an HCDR2 that comprises, or consists essentially of, or yet further consists of the amino acid sequence of SEQ ID NO: 54, and an HCDR3 that comprises, or consists essentially of, or yet further consists of the amino acid sequence of SEQ ID NO: 55;
(SCT-Pa05) an HCDR1 that comprises, or consists essentially of, or yet further consists of the amino acid sequence of SEQ ID NO: 56, an HCDR2 that comprises, or consists essentially of, or yet further consists of the amino acid sequence of SEQ ID NO: 57, and an HCDR3 that comprises, or consists essentially of, or yet further consists of the amino acid sequence of SEQ ID NO: 58;
(SCT-Pa06) an HCDR1 that comprises, or consists essentially of, or yet further consists of the amino acid sequence of SEQ ID NO: 59, an HCDR2 that comprises, or consists essentially of, or yet further consists of the amino acid sequence of SEQ ID NO: 60, and an HCDR3 that comprises, or consists essentially of, or yet further consists of the amino acid sequence of SEQ ID NO: 61;
(SCT-Pa07) an HCDR1 that comprises, or consists essentially of, or yet further consists of the amino acid sequence of SEQ ID NO: 50, an HCDR2 that comprises, or consists essentially of, or yet further consists of the amino acid sequence of SEQ ID NO: 62, and an HCDR3 that comprises, or consists essentially of, or yet further consists of the amino acid sequence of SEQ ID NO: 63;
(SCT-Pa08) an HCDR1 that comprises, or consists essentially of, or yet further consists of the amino acid sequence of SEQ ID NO: 64, an HCDR2 that comprises, or consists essentially of, or yet further consists of the amino acid sequence of SEQ ID NO: 65, and an HCDR3 that comprises, or consists essentially of, or yet further consists of the amino acid sequence of SEQ ID NO: 66;
(SCT-Pa09) an HCDR1 that comprises, or consists essentially of, or yet further consists of the amino acid sequence of SEQ ID NO: 67, an HCDR2 that comprises, or consists essentially of, or yet further consists of the amino acid sequence of SEQ ID NO: 68, and an HCDR3 that comprises, or consists essentially of, or yet further consists of the amino acid sequence of SEQ ID NO: 69;
(SCT-Pa10) an HCDR1 that comprises, or consists essentially of, or yet further consists of the amino acid sequence of SEQ ID NO: 70, an HCDR2 that comprises, or consists essentially of, or yet further consists of the amino acid sequence of SEQ ID NO: 71, and an HCDR3 that comprises, or consists essentially of, or yet further consists of the amino acid sequence of SEQ ID NO: 72;
(SCT-Pa11) an HCDR1 that comprises, or consists essentially of, or yet further consists of the amino acid sequence of SEQ ID NO: 73, an HCDR2 that comprises, or consists essentially of, or yet further consists of the amino acid sequence of SEQ ID NO: 54, and an HCDR3 that comprises, or consists essentially of, or yet further consists of the amino acid sequence of SEQ ID NO: 74;
(SCT-Pa12) an HCDR1 that comprises, or consists essentially of, or yet further consists of the amino acid sequence of SEQ ID NO: 75, an HCDR2 that comprises, or consists essentially of, or yet further consists of the amino acid sequence of SEQ ID NO: 76, and an HCDR3 that comprises, or consists essentially of, or yet further consists of the amino acid sequence of SEQ ID NO: 77;
(SCT-Pa13) an HCDR1 that comprises, or consists essentially of, or yet further consists of the amino acid sequence of SEQ ID NO: 78, an HCDR2 that comprises, or consists essentially of, or yet further consists of the amino acid sequence of SEQ ID NO: 79, and an HCDR3 that comprises, or consists essentially of, or yet further consists of the amino acid sequence of SEQ ID NO: 80;
(SCT-Pa14) an HCDR1 that comprises, or consists essentially of, or yet further consists of the amino acid sequence of SEQ ID NO: 81, an HCDR2 that comprises, or consists essentially of, or yet further consists of the amino acid sequence of SEQ ID NO: 82, and an HCDR3 that comprises, or consists essentially of, or yet further consists of the amino acid sequence of SEQ ID NO: 83;
(SCT-Pa15) an HCDR1 that comprises, or consists essentially of, or yet further consists of the amino acid sequence of SEQ ID NO: 84, an HCDR2 that comprises, or consists essentially of, or yet further consists of the amino acid sequence of SEQ ID NO: 79, and an HCDR3 that comprises, or consists essentially of, or yet further consists of the amino acid sequence of SEQ ID NO: 85;
(SCT-Pa16) an HCDR1 that comprises, or consists essentially of, or yet further consists of the amino acid sequence of SEQ ID NO: 86, an HCDR2 that comprises, or consists essentially of, or yet further consists of the amino acid sequence of SEQ ID NO: 87, and an HCDR3 that comprises, or consists essentially of, or yet further consists of the amino acid sequence of SEQ ID NO: 88;
(SCT-Pa17) an HCDR1 that comprises, or consists essentially of, or yet further consists of the amino acid sequence of SEQ ID NO: 89, an HCDR2 that comprises, or consists essentially of, or yet further consists of the amino acid sequence of SEQ ID NO: 90, and an HCDR3 that comprises, or consists essentially of, or yet further consists of the amino acid sequence of SEQ ID NO: 91;
(SCT-Pa18) an HCDR1 that comprises, or consists essentially of, or yet further consists of the amino acid sequence of SEQ ID NO: 86, an HCDR2 that comprises, or consists essentially of, or yet further consists of the amino acid sequence of SEQ ID NO: 92, and an HCDR3 that comprises, or consists essentially of, or yet further consists of the amino acid sequence of SEQ ID NO: 93;
(SCT-Pa19) an HCDR1 that comprises, or consists essentially of, or yet further consists of the amino acid sequence of SEQ ID NO: 94, an HCDR2 that comprises, or consists essentially of, or yet further consists of the amino acid sequence of SEQ ID NO: 95, and an HCDR3 that comprises, or consists essentially of, or yet further consists of the amino acid sequence of SEQ ID NO: 96; or
(SCT-Pa20) an HCDR1 that comprises, or consists essentially of, or yet further consists of the amino acid sequence of SEQ ID NO: 97, an HCDR2 that comprises, or consists essentially of, or yet further consists of the amino acid sequence of SEQ ID NO: 98, and an HCDR3 that comprises, or consists essentially of, or yet further consists of the amino acid sequence of SEQ ID NO: 99.

Additionally or alternatively, the antibody or antigen binding fragment thereof comprises, or consists essentially of, or yet further consists of one or more of:
(SCT-Pa01) a light chain (LC) complementarity determining region (CDR) 1 (LCDR1) that comprises, or consists essentially of, or yet further consists of the amino acid sequence of SEQ ID NO: 100, an LC CDR 2 (LCDR2) that comprises, or consists essentially of, or yet further consists of the amino acid sequence of SEQ ID NO: 101, and an LC CDR 3 (LCDR3) that comprises, or consists essentially of, or yet further consists of the amino acid sequence of SEQ ID NO: 102;
(SCT-Pa02) an LCDR1 that comprises, or consists essentially of, or yet further consists of the amino acid sequence of SEQ ID NO: 103, an LCDR2 that comprises, or consists essentially of, or yet further consists of the amino acid sequence of SEQ ID NO: 104, and an LCDR3 that comprises, or consists essentially of, or yet further consists of the amino acid sequence of SEQ ID NO: 105;
(SCT-Pa03) an LCDR1 that comprises, or consists essentially of, or yet further consists of the amino acid sequence of SEQ ID NO: 106, an LCDR2 that comprises, or consists essentially of, or yet further consists of the amino acid sequence of SEQ ID NO: 107, and an LCDR3 that comprises, or consists essentially of, or yet further consists of the amino acid sequence of SEQ ID NO: 108;
(SCT-Pa04) an LCDR1 that comprises, or consists essentially of, or yet further consists of the amino acid sequence of SEQ ID NO: 109, an LCDR2 that comprises, or consists essentially of, or yet further consists of the amino acid sequence of SEQ ID NO: 110, and an LCDR3 that comprises, or consists essentially of, or yet further consists of the amino acid sequence of SEQ ID NO: 111;
(SCT-Pa05) an LCDR1 that comprises, or consists essentially of, or yet further consists of the amino acid sequence of SEQ ID NO: 112, an LCDR2 that comprises, or consists essentially of, or yet further consists of the amino acid sequence of SEQ ID NO: 113, and an LCDR3 that comprises, or consists essentially of, or yet further consists of the amino acid sequence of SEQ ID NO: 114;
(SCT-Pa06) an LCDR1 that comprises, or consists essentially of, or yet further consists of the amino acid sequence of SEQ ID NO: 115, an LCDR2 that comprises, or consists essentially of, or yet further consists of the amino acid sequence of SEQ ID NO: 101, and an LCDR3 that comprises, or consists essentially of, or yet further consists of the amino acid sequence of SEQ ID NO: 116;
(SCT-Pa07) an LCDR1 that comprises, or consists essentially of, or yet further consists of the amino acid sequence of SEQ ID NO: 117, an LCDR2 that comprises, or consists essentially of, or yet further consists of the amino acid sequence of SEQ ID NO: 118, and an LCDR3 that comprises, or consists essentially of, or yet further consists of the amino acid sequence of SEQ ID NO: 119;
(SCT-Pa08) an LCDR1 that comprises, or consists essentially of, or yet further consists of the amino acid sequence of SEQ ID NO: 120, an LCDR2 that comprises, or consists essentially of, or yet further consists of the amino acid sequence of SEQ ID NO: 121, and an LCDR3 that comprises, or consists essentially of, or yet further consists of the amino acid sequence of SEQ ID NO: 122;
(SCT-Pa09) an LCDR1 that comprises, or consists essentially of, or yet further consists of the amino acid sequence of SEQ ID NO: 123, an LCDR2 that comprises, or consists essentially of, or yet further consists of the amino acid sequence of SEQ ID NO: 104, and an LCDR3 that comprises, or consists essentially of, or yet further consists of the amino acid sequence of SEQ ID NO: 124;
(SCT-Pa10) an LCDR1 that comprises, or consists essentially of, or yet further consists of the amino acid sequence of SEQ ID NO: 125, an LCDR2 that comprises, or consists essentially of, or yet further consists of the amino acid sequence of SEQ ID NO: 118, and an LCDR3 that comprises, or consists essentially of, or yet further consists of the amino acid sequence of SEQ ID NO: 126;
(SCT-Pa11) an LCDR1 that comprises, or consists essentially of, or yet further consists of the amino acid sequence of SEQ ID NO: 127, an LCDR2 that comprises, or consists essentially of, or yet further consists of the amino acid sequence of SEQ ID NO: 128, and an LCDR3 that comprises, or consists essentially of, or yet further consists of the amino acid sequence of SEQ ID NO: 129;
(SCT-Pa12) an LCDR1 that comprises, or consists essentially of, or yet further consists of the amino acid sequence of SEQ ID NO: 130, an LCDR2 that comprises, or consists essentially of, or yet further consists of the amino acid sequence of SEQ ID NO: 118, and an LCDR3 that comprises, or consists essentially of, or yet further consists of the amino acid sequence of SEQ ID NO: 126;
(SCT-Pa13) an LCDR1 that comprises, or consists essentially of, or yet further consists of the amino acid sequence of SEQ ID NO: 131, an LCDR2 that comprises, or consists essentially of, or yet further consists of the amino acid sequence of SEQ ID NO: 132, and an LCDR3 that comprises, or consists essentially of, or yet further consists of the amino acid sequence of SEQ ID NO: 133;
(SCT-Pa14) an LCDR1 that comprises, or consists essentially of, or yet further consists of the amino acid sequence of SEQ ID NO: 117, an LCDR2 that comprises, or consists essentially of, or yet further consists of the amino acid sequence of SEQ ID NO: 118, and an LCDR3 that comprises, or consists essentially of, or yet further consists of the amino acid sequence of SEQ ID NO: 134;
(SCT-Pa15) an LCDR1 that comprises, or consists essentially of, or yet further consists of the amino acid sequence of SEQ ID NO: 135, an LCDR2 that comprises, or consists essentially of, or yet further consists of the amino acid sequence of SEQ ID NO: 136, and an LCDR3 that comprises, or consists essentially of, or yet further consists of the amino acid sequence of SEQ ID NO: 137;
(SCT-Pa16) an LCDR1 that comprises, or consists essentially of, or yet further consists of the amino acid sequence of SEQ ID NO: 138, an LCDR2 that comprises, or consists essentially of, or yet further consists of the amino acid sequence of SEQ ID NO: 139, and an LCDR3 that comprises, or consists essentially of, or yet further consists of the amino acid sequence of SEQ ID NO: 140;
(SCT-Pa17) an LCDR1 that comprises, or consists essentially of, or yet further consists of the amino acid sequence of SEQ ID NO: 141, an LCDR2 that comprises, or consists essentially of, or yet further consists of the amino acid sequence of SEQ ID NO: 107, and an LCDR3 that comprises, or consists essentially of, or yet further consists of the amino acid sequence of SEQ ID NO: 142;
(SCT-Pa18) an LCDR1 that comprises, or consists essentially of, or yet further consists of the amino acid sequence of SEQ ID NO: 130, an LCDR2 that comprises, or consists essentially of, or yet further consists of the amino acid sequence of SEQ ID NO: 118, and an LCDR3 that comprises, or consists essentially of, or yet further consists of the amino acid sequence of SEQ ID NO: 126;
(SCT-Pa19) an LCDR1 that comprises, or consists essentially of, or yet further consists of the amino acid sequence of SEQ ID NO: 143, an LCDR2 that comprises, or consists essentially of, or yet further consists of the amino acid sequence of SEQ ID NO: 139, and an LCDR3 that comprises, or consists essentially of, or yet further consists of the amino acid sequence of SEQ ID NO: 144; or
(SCT-Pa20) an LCDR1 that comprises, or consists essentially of, or yet further consists of the amino acid sequence of SEQ ID NO: 145, an LCDR2 that comprises, or consists essentially of, or yet further consists of the amino acid sequence of SEQ ID NO: 146, and an LCDR3 that comprises, or consists essentially of, or yet further consists of the amino acid sequence of SEQ ID NO: 147.

In some embodiments, the antibody or antigen binding fragment thereof comprises, or consists essentially of, or yet further consists of one or more of:
(SCT-Pa01) any one or any two or any three or any four or any five or all six of: a heavy chain (HC) complementarity determining region (CDR) 1 (HCDR1) that comprises, or consists essentially of, or yet further consists of the amino acid sequence of SEQ ID NO: 44, an HC CDR 2 (HCDR2) that comprises, or consists essentially of, or yet further consists of the amino acid sequence of SEQ ID NO: 45, an HC CDR 3 (HCDR3) that comprises, or consists essentially of, or yet further consists of the amino acid sequence of SEQ ID NO: 46, a light chain (LC) complementarity determining region (CDR) 1 (LCDR1) that comprises, or consists essentially of, or yet further consists of the amino acid sequence of SEQ ID NO: 100, an LC CDR 2 (LCDR2) that comprises, or consists essentially of, or yet further consists of the amino acid sequence of SEQ ID NO: 101, or an LC CDR 3 (LCDR3) that comprises, or consists essentially of, or yet further consists of the amino acid sequence of SEQ ID NO: 102;
(SCT-Pa02) any one or any two or any three or any four or any five or all six of: an HCDR1 that comprises, or consists essentially of, or yet further consists of the amino acid sequence of SEQ ID NO: 47, an HCDR2 that comprises, or consists essentially of, or yet further consists of the amino acid sequence of SEQ ID NO: 48, an HCDR3 that comprises, or consists essentially of, or yet further consists of the amino acid sequence of SEQ ID NO: 49, an LCDR1 that comprises, or consists essentially of, or yet further consists of the amino acid sequence of SEQ ID NO: 103, an LCDR2 that comprises, or consists essentially of, or yet further consists of the amino acid sequence of SEQ ID NO: 104, or an LCDR3 that comprises, or consists essentially of, or yet further consists of the amino acid sequence of SEQ ID NO: 105;
(SCT-Pa03) any one or any two or any three or any four or any five or all six of: an HCDR1 that comprises, or consists essentially of, or yet further consists of the amino acid sequence of SEQ ID NO: 50, an HCDR2 that comprises, or consists essentially of, or yet further consists of the amino acid sequence of SEQ ID NO: 51, an HCDR3 that comprises, or consists essentially of, or yet further consists of the amino acid sequence of SEQ ID NO: 52, an LCDR1 that comprises, or consists essentially of, or yet further consists of the amino acid sequence of SEQ ID NO: 106, an LCDR2 that comprises, or consists essentially of, or yet further consists of the amino acid sequence of SEQ ID NO: 107, or an LCDR3 that comprises, or consists essentially of, or yet further consists of the amino acid sequence of SEQ ID NO: 108;
(SCT-Pa04) any one or any two or any three or any four or any five or all six of: an HCDR1 that comprises, or consists essentially of, or yet further consists of the amino acid sequence of SEQ ID NO: 53, an HCDR2 that comprises, or consists essentially of, or yet further consists of the amino acid sequence of SEQ ID NO: 54, an HCDR3 that comprises, or consists essentially of, or yet further consists of the amino acid sequence of SEQ ID NO: 55, an LCDR1 that comprises, or consists essentially of, or yet further consists of the amino acid sequence of SEQ ID NO: 109, an LCDR2 that comprises, or consists essentially of, or yet further consists of the amino acid sequence of SEQ ID NO: 110, or an LCDR3 that comprises, or consists essentially of, or yet further consists of the amino acid sequence of SEQ ID NO: 111;
(SCT-Pa05) any one or any two or any three or any four or any five or all six of: an HCDR1 that comprises, or consists essentially of, or yet further consists of the amino acid sequence of SEQ ID NO: 56, an HCDR2 that comprises, or consists essentially of, or yet further consists of the amino acid sequence of SEQ ID NO: 57, an HCDR3 that comprises, or consists essentially of, or yet further consists of the amino acid sequence of SEQ ID NO: 58, an LCDR1 that comprises, or consists essentially of, or yet further consists of the amino acid sequence of SEQ ID NO: 112, an LCDR2 that comprises, or consists essentially of, or yet further consists of the amino acid sequence of SEQ ID NO: 113, or an LCDR3 that comprises, or consists essentially of, or yet further consists of the amino acid sequence of SEQ ID NO: 114;
(SCT-Pa06) any one or any two or any three or any four or any five or all six of: an HCDR1 that comprises, or consists essentially of, or yet further consists of the amino acid sequence of SEQ ID NO: 59, an HCDR2 that comprises, or consists essentially of, or yet further consists of the amino acid sequence of SEQ ID NO: 60, an HCDR3 that comprises, or consists essentially of, or yet further consists of the amino acid sequence of SEQ ID NO: 61, an LCDR1 that comprises, or consists essentially of, or yet further consists of the amino acid sequence of SEQ ID NO: 115, an LCDR2 that comprises, or consists essentially of, or yet further consists of the amino acid sequence of SEQ ID NO: 101, or an LCDR3 that comprises, or consists essentially of, or yet further consists of the amino acid sequence of SEQ ID NO: 116;
(SCT-Pa07) any one or any two or any three or any four or any five or all six of: an HCDR1 that comprises, or consists essentially of, or yet further consists of the amino acid sequence of SEQ ID NO: 50, an HCDR2 that comprises, or consists essentially of, or yet further consists of the amino acid sequence of SEQ ID NO: 62, an HCDR3 that comprises, or consists essentially of, or yet further consists of the amino acid sequence of SEQ ID NO: 63, an LCDR1 that comprises, or consists essentially of, or yet further consists of the amino acid sequence of SEQ ID NO: 117, an LCDR2 that comprises, or consists essentially of, or yet further consists of the amino acid sequence of SEQ ID NO: 118, or an LCDR3 that comprises, or consists essentially of, or yet further consists of the amino acid sequence of SEQ ID NO: 119;
(SCT-Pa08) any one or any two or any three or any four or any five or all six of: an HCDR1 that comprises, or consists essentially of, or yet further consists of the amino acid sequence of SEQ ID NO: 64, an HCDR2 that comprises, or consists essentially of, or yet further consists of the amino acid sequence of SEQ ID NO: 65, an HCDR3 that comprises, or consists essentially of, or yet further consists of the amino acid sequence of SEQ ID NO: 66, an LCDR1 that comprises, or consists essentially of, or yet further consists of the amino acid sequence of SEQ ID NO: 120, an LCDR2 that comprises, or consists essentially of, or yet further consists of the amino acid sequence of SEQ ID NO: 121, or an LCDR3 that comprises, or consists essentially of, or yet further consists of the amino acid sequence of SEQ ID NO: 122;
(SCT-Pa09) any one or any two or any three or any four or any five or all six of: an HCDR1 that comprises, or consists essentially of, or yet further consists of the amino acid sequence of SEQ ID NO: 67, an HCDR2 that comprises, or consists essentially of, or yet further consists of the amino acid sequence of SEQ ID NO: 68, an HCDR3 that comprises, or consists essentially of, or yet further consists of the amino acid sequence of SEQ ID NO: 69, an LCDR1 that comprises, or consists essentially of, or yet further consists of the amino acid sequence of SEQ ID NO: 123, an LCDR2 that comprises, or consists essentially of, or yet further consists of the amino acid sequence of SEQ ID NO: 104, or an LCDR3 that comprises, or consists essentially of, or yet further consists of the amino acid sequence of SEQ ID NO: 124;
(SCT-Pa10) any one or any two or any three or any four or any five or all six of: an HCDR1 that comprises, or consists essentially of, or yet further consists of the amino acid sequence of SEQ ID NO: 70, an HCDR2 that comprises, or consists essentially of, or yet further consists of the amino acid sequence of SEQ ID NO: 71, an HCDR3 that comprises, or consists essentially of, or yet further consists of the amino acid sequence of SEQ ID NO: 72, an LCDR1 that comprises, or consists essentially of, or yet further consists of the amino acid sequence of SEQ ID NO: 125, an LCDR2 that comprises, or consists essentially of, or yet further consists of the amino acid sequence of SEQ ID NO: 118, or an LCDR3 that comprises, or consists essentially of, or yet further consists of the amino acid sequence of SEQ ID NO: 126;
(SCT-Pa11) any one or any two or any three or any four or any five or all six of: an HCDR1 that comprises, or consists essentially of, or yet further consists of the amino acid sequence of SEQ ID NO: 73, an HCDR2 that comprises, or consists essentially of, or yet further consists of the amino acid sequence of SEQ ID NO: 54, an HCDR3 that comprises, or consists essentially of, or yet further consists of the amino acid sequence of SEQ ID NO: 74, an LCDR1 that comprises, or consists essentially of, or yet further consists of the amino acid sequence of SEQ ID NO: 127, an LCDR2 that comprises, or consists essentially of, or yet further consists of the amino acid sequence of SEQ ID NO: 128, or an LCDR3 that comprises, or consists essentially of, or yet further consists of the amino acid sequence of SEQ ID NO: 129;
(SCT-Pa12) any one or any two or any three or any four or any five or all six of: an HCDR1 that comprises, or consists essentially of, or yet further consists of the amino acid sequence of SEQ ID NO: 75, an HCDR2 that comprises, or consists essentially of, or yet further consists of the amino acid sequence of SEQ ID NO: 76, an HCDR3 that comprises, or consists essentially of, or yet further consists of the amino acid sequence of SEQ ID NO: 77, an LCDR1 that comprises, or consists essentially of, or yet further consists of the amino acid sequence of SEQ ID NO: 130, an LCDR2 that comprises, or consists essentially of, or yet further consists of the amino acid sequence of SEQ ID NO: 118, or an LCDR3 that comprises, or consists essentially of, or yet further consists of the amino acid sequence of SEQ ID NO: 126;
(SCT-Pa13) any one or any two or any three or any four or any five or all six of: an HCDR1 that comprises, or consists essentially of, or yet further consists of the amino acid sequence of SEQ ID NO: 78, an HCDR2 that comprises, or consists essentially of, or yet further consists of the amino acid sequence of SEQ ID NO: 79, an HCDR3 that comprises, or consists essentially of, or yet further consists of the amino acid sequence of SEQ ID NO: 80, an LCDR1 that comprises, or consists essentially of, or yet further consists of the amino acid sequence of SEQ ID NO: 131, an LCDR2 that comprises, or consists essentially of, or yet further consists of the amino acid sequence of SEQ ID NO: 132, or an LCDR3 that comprises, or consists essentially of, or yet further consists of the amino acid sequence of SEQ ID NO: 133;
(SCT-Pa14) any one or any two or any three or any four or any five or all six of: an HCDR1 that comprises, or consists essentially of, or yet further consists of the amino acid sequence of SEQ ID NO: 81, an HCDR2 that comprises, or consists essentially of, or yet further consists of the amino acid sequence of SEQ ID NO: 82, an HCDR3 that comprises, or consists essentially of, or yet further consists of the amino acid sequence of SEQ ID NO: 83, an LCDR1 that comprises, or consists essentially of, or yet further consists of the amino acid sequence of SEQ ID NO: 117, an LCDR2 that comprises, or consists essentially of, or yet further consists of the amino acid sequence of SEQ ID NO: 118, or an LCDR3 that comprises, or consists essentially of, or yet further consists of the amino acid sequence of SEQ ID NO: 134;
(SCT-Pa15) any one or any two or any three or any four or any five or all six of: an HCDR1 that comprises, or consists essentially of, or yet further consists of the amino acid sequence of SEQ ID NO: 84, an HCDR2 that comprises, or consists essentially of, or yet further consists of the amino acid sequence of SEQ ID NO: 79, an HCDR3 that comprises, or consists essentially of, or yet further consists of the amino acid sequence of SEQ ID NO: 85, an LCDR1 that comprises, or consists essentially of, or yet further consists of the amino acid sequence of SEQ ID NO: 135, an LCDR2 that comprises, or consists essentially of, or yet further consists of the amino acid sequence of SEQ ID NO: 136, or an LCDR3 that comprises, or consists essentially of, or yet further consists of the amino acid sequence of SEQ ID NO: 137;
(SCT-Pa16) any one or any two or any three or any four or any five or all six of: an HCDR1 that comprises, or consists essentially of, or yet further consists of the amino acid sequence of SEQ ID NO: 86, an HCDR2 that comprises, or consists essentially of, or yet further consists of the amino acid sequence of SEQ ID NO: 87, an HCDR3 that comprises, or consists essentially of, or yet further consists of the amino acid sequence of SEQ ID NO: 88, an LCDR1 that comprises, or consists essentially of, or yet further consists of the amino acid sequence of SEQ ID NO: 138, an LCDR2 that comprises, or consists essentially of, or yet further consists of the amino acid sequence of SEQ ID NO: 139, or an LCDR3 that comprises, or consists essentially of, or yet further consists of the amino acid sequence of SEQ ID NO: 140;
(SCT-Pa17) any one or any two or any three or any four or any five or all six of: an HCDR1 that comprises, or consists essentially of, or yet further consists of the amino acid sequence of SEQ ID NO: 89, an HCDR2 that comprises, or consists essentially of, or yet further consists of the amino acid sequence of SEQ ID NO: 90, an HCDR3 that comprises, or consists essentially of, or yet further consists of the amino acid sequence of SEQ ID NO: 91, an LCDR1 that comprises, or consists essentially of, or yet further consists of the amino acid sequence of SEQ ID NO: 141, an LCDR2 that comprises, or consists essentially of, or yet further consists of the amino acid sequence of SEQ ID NO: 107, or an LCDR3 that comprises, or consists essentially of, or yet further consists of the amino acid sequence of SEQ ID NO: 142;
(SCT-Pa18) any one or any two or any three or any four or any five or all six of: an HCDR1 that comprises, or consists essentially of, or yet further consists of the amino acid sequence of SEQ ID NO: 86, an HCDR2 that comprises, or consists essentially of, or yet further consists of the amino acid sequence of SEQ ID NO: 92, an HCDR3 that comprises, or consists essentially of, or yet further consists of the amino acid sequence of SEQ ID NO: 93, an LCDR1 that comprises, or consists essentially of, or yet further consists of the amino acid sequence of SEQ ID NO: 130, an LCDR2 that comprises, or consists essentially of, or yet further consists of the amino acid sequence of SEQ ID NO: 118, or an LCDR3 that comprises, or consists essentially of, or yet further consists of the amino acid sequence of SEQ ID NO: 126;
(SCT-Pa19) any one or any two or any three or any four or any five or all six of: an HCDR1 that comprises, or consists essentially of, or yet further consists of the amino acid sequence of SEQ ID NO: 94, an HCDR2 that comprises, or consists essentially of, or yet further consists of the amino acid sequence of SEQ ID NO: 95, an HCDR3 that comprises, or consists essentially of, or yet further consists of the amino acid sequence of SEQ ID NO: 96, an LCDR1 that comprises, or consists essentially of, or yet further consists of the amino acid sequence of SEQ ID NO: 143, an LCDR2 that comprises, or consists essentially of, or yet further consists of the amino acid sequence of SEQ ID NO: 139, or an LCDR3 that comprises, or consists essentially of, or yet further consists of the amino acid sequence of SEQ ID NO: 144; or
(SCT-Pa20) any one or any two or any three or any four or any five or all six of: an HCDR1 that comprises, or consists essentially of, or yet further consists of the amino acid sequence of SEQ ID NO: 97, an HCDR2 that comprises, or consists essentially of, or yet further consists of the amino acid sequence of SEQ ID NO: 98, an HCDR3 that comprises, or consists essentially of, or yet further consists of the amino acid sequence of SEQ ID NO: 99, an LCDR1 that comprises, or consists essentially of, or yet further consists of the amino acid sequence of SEQ ID NO: 145, an LCDR2 that comprises, or consists essentially of, or yet further consists of the amino acid sequence of SEQ ID NO: 146, or an LCDR3 that comprises, or consists essentially of, or yet further consists of the amino acid sequence of SEQ ID NO: 147.

In some embodiments, the antibody or an antigen binding fragment thereof comprises, or consists essentially of, or yet further consists of one or more of:
(SCT-Pa01) any one or any two or any three or any four or any five or all six of: complementarity determining regions (CDRs) of a heavy chain variable domain that comprises SEQ ID NO: 4, or CDRs of a light chain variable domain that comprises SEQ ID NO: 24;
(SCT-Pa02) any one or any two or any three or any four or any five or all six of: CDRs of a heavy chain variable domain that comprises SEQ ID NO: 5, or CDRs of a light chain variable domain that comprises SEQ ID NO: 25;
(SCT-Pa03) any one or any two or any three or any four or any five or all six of: CDRs of a heavy chain variable domain that comprises SEQ ID NO: 6, or CDRs of a light chain variable domain that comprises SEQ ID NO: 26;
(SCT-Pa04) any one or any two or any three or any four or any five or all six of: CDRs of a heavy chain variable domain that comprises SEQ ID NO: 7, or CDRs of a light chain variable domain that comprises SEQ ID NO: 27;
(SCT-Pa05) any one or any two or any three or any four or any five or all six of: CDRs of a heavy chain variable domain that comprises SEQ ID NO: 8, or CDRs of a light chain variable domain that comprises SEQ ID NO: 28;
(SCT-Pa06) any one or any two or any three or any four or any five or all six of: CDRs of a heavy chain variable domain that comprises SEQ ID NO: 9, or CDRs of a light chain variable domain that comprises SEQ ID NO: 29;
(SCT-Pa07) any one or any two or any three or any four or any five or all six of: CDRs of a heavy chain variable domain that comprises SEQ ID NO: 10, or CDRs of a light chain variable domain that comprises SEQ ID NO: 30;
(SCT-Pa08) any one or any two or any three or any four or any five or all six of: CDRs of a heavy chain variable domain that comprises SEQ ID NO: 11, or CDRs of a light chain variable domain that comprises SEQ ID NO: 31;
(SCT-Pa09) any one or any two or any three or any four or any five or all six of: CDRs of a heavy chain variable domain that comprises SEQ ID NO: 12, or CDRs of a light chain variable domain that comprises SEQ ID NO: 32;
(SCT-Pa10) any one or any two or any three or any four or any five or all six of: CDRs of a heavy chain variable domain that comprises SEQ ID NO: 13, or CDRs of a light chain variable domain that comprises SEQ ID NO: 33;
(SCT-Pa11) any one or any two or any three or any four or any five or all six of: CDRs of a heavy chain variable domain that comprises SEQ ID NO: 14, or CDRs of a light chain variable domain that comprises SEQ ID NO: 34;
(SCT-Pa12) any one or any two or any three or any four or any five or all six of: CDRs of a heavy chain variable domain that comprises SEQ ID NO: 15, or CDRs of a light chain variable domain that comprises SEQ ID NO: 35;
(SCT-Pa13) any one or any two or any three or any four or any five or all six of: CDRs of a heavy chain variable domain that comprises SEQ ID NO: 16, or CDRs of a light chain variable domain that comprises SEQ ID NO: 36;
(SCT-Pa14) any one or any two or any three or any four or any five or all six of: CDRs of a heavy chain variable domain that comprises SEQ ID NO: 17, or CDRs of a light chain variable domain that comprises SEQ ID NO: 37;
(SCT-Pa15) any one or any two or any three or any four or any five or all six of: CDRs of a heavy chain variable domain that comprises SEQ ID NO: 18, or CDRs of a light chain variable domain that comprises SEQ ID NO: 38;
(SCT-Pa16) any one or any two or any three or any four or any five or all six of: CDRs of a heavy chain variable domain that comprises SEQ ID NO: 19, or CDRs of a light chain variable domain that comprises SEQ ID NO: 39;
(SCT-Pa17) any one or any two or any three or any four or any five or all six of: CDRs of a heavy chain variable domain that comprises SEQ ID NO: 20, or CDRs of a light chain variable domain that comprises SEQ ID NO: 40;
(SCT-Pa18) any one or any two or any three or any four or any five or all six of: CDRs of a heavy chain variable domain that comprises SEQ ID NO: 21, or CDRs of a light chain variable domain that comprises SEQ ID NO: 41;
(SCT-Pa19) any one or any two or any three or any four or any five or all six of: CDRs of a heavy chain variable domain that comprises SEQ ID NO: 22, or CDRs of a light chain variable domain that comprises SEQ ID NO: 42; or
(SCT-Pa20) any one or any two or any three or any four or any five or all six of: CDRs of a heavy chain variable domain that comprises SEQ ID NO: 23, or CDRs of a light chain variable domain that comprises SEQ ID NO: 43.

### Antibody Variable Domain Sequences

The antibodies of the disclosure can comprise, or consist essentially of, or yet further consist of any one or more of the heavy chain variable domain sequences of SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23. Any one of the heavy chain variable domain sequences can comprise, or consist essentially of, or yet further consist of SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

The antibodies of the disclosure can comprise, or consist essentially of, or yet further consist of any one or more of the light chain variable domain sequences of SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, or SEQ ID NO:43. The light chain variable domain sequences can comprise, or consist essentially of, or yet further consist of SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, or SEQ ID NO:43.

The disclosure also provides a variable domain sequence comprising, or consisting essentially of, or yet further consisting of a sequence that is at least 80%, at least 85%, at least 90%, or at least 95% identical to a sequence selected from SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23. The disclosure also provides a variable domain sequence comprising, or consisting essentially of, or yet further consisting of a sequence that is at least 80%, at least 85%, at least 90%, or at least 95% identical to a sequence selected from SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, or SEQ ID NO:43.

The disclosure also provides antibodies comprising, or consisting essentially of, or yet further consisting of a heavy chain variable domain sequence that is at least 80%, at least 85%, at least 90%, or at least 95% identical to SEQ ID NO:4 and a light chain variable domain sequence that is at least 80%, at least 85%, at least 90%, or at least 95% identical to SEQ ID NO:24. The disclosure also provides antibodies comprising, or consisting essentially of, or yet further consisting of a heavy chain variable domain sequence that is at least 80%, at least 85%, at least 90%, or at least 95% identical to SEQ ID NO:5 and a light chain variable domain sequence that is at least 80%, at least 85%, at least 90%, or at least 95% identical to SEQ ID NO:25. The disclosure also provides antibodies comprising, or consisting essentially of, or yet further consisting of a heavy chain variable domain sequence that is at least 80%, at least 85%, at least 90%, or at least 95% identical to SEQ ID NO:6 and a light chain variable domain sequence that is at least 80%, at least 85%, at least 90%, or at least 95% identical to SEQ ID NO:26. The disclosure also provides antibodies comprising, or consisting essentially of, or yet further consisting of a heavy chain variable domain sequence that is at least 80%, at least 85%, at least 90%, or at least 95% identical to SEQ ID NO:7 and a light chain variable domain sequence that is at least 80%, at least 85%, at least 90%, or at least 95% identical to SEQ ID NO:27. The disclosure also provides antibodies comprising, or consisting essentially of, or yet further consisting of a heavy chain variable domain sequence that is at least 80%, at least 85%, at least 90%, or at least 95% identical to SEQ ID NO:8 and a light chain variable domain sequence that is at least 80%, at least 85%, at least 90%, or at least 95% identical to SEQ ID NO:28. The disclosure also provides antibodies comprising, or consisting essentially of, or yet further consisting of a heavy chain variable domain sequence that is at least 80%, at least 85%, at least 90%, or at least 95% identical to SEQ ID NO:9 and a light chain variable domain sequence that is at least 80%, at least 85%, at least 90%, or at least 95% identical to SEQ ID NO:29. The disclosure also provides antibodies comprising, or consisting essentially of, or yet further consisting of a heavy chain variable domain sequence that is at least 80%, at least 85%, at least 90%, or at least 95% identical to SEQ ID NO:10 and a light chain variable domain sequence that is at least 80%, at least 85%, at least 90%, or at least 95% identical to SEQ ID NO:30. The disclosure also provides antibodies comprising, or consisting essentially of, or yet further consisting of a heavy chain variable domain sequence that is at least 80%, at least 85%, at least 90%, or at least 95% identical to SEQ ID NO:11 and a light chain variable domain sequence that is at least 80%, at least 85%, at least 90%, or at least 95% identical to SEQ ID NO:31. The disclosure also provides antibodies comprising, or consisting essentially of, or yet further consisting of a heavy chain variable domain sequence that is at least 80%, at least 85%, at least 90%, or at least 95% identical to SEQ ID NO:12 and a light chain variable domain sequence that is at least 80%, at least 85%, at least 90%, or at least 95% identical to SEQ ID NO:32. The disclosure also provides antibodies comprising, or consisting essentially of, or yet further consisting of a heavy chain variable domain sequence that is at least 80%, at least 85%, at least 90%, or at least 95% identical to SEQ ID NO:13 and a light chain variable domain sequence that is at least 80%, at least 85%, at least 90%, or at least 95% identical to SEQ ID NO:33. The disclosure also provides antibodies comprising, or consisting essentially of, or yet further consisting of a heavy chain variable domain sequence that is at least 80%, at least 85%, at least 90%, or at least 95% identical to SEQ ID NO:14 and a light chain variable domain sequence that is at least 80%, at least 85%, at least 90%, or at least 95% identical to SEQ ID NO:34. The disclosure also provides antibodies comprising, or consisting essentially of, or yet further consisting of a heavy chain variable domain sequence that is at least 80%, at least 85%, at least 90%, or at least 95% identical to SEQ ID NO:15 and a light chain variable domain sequence that is at least 80%, at least 85%, at least 90%, or at least 95% identical to SEQ ID NO:35. The disclosure also provides antibodies comprising, or consisting essentially of, or yet further consisting of a heavy chain variable domain sequence that is at least 80%, at least 85%, at least 90%, or at least 95% identical to SEQ ID NO:16 and a light chain variable domain sequence that is at least 80%, at least 85%, at least 90%, or at least 95% identical to SEQ ID NO:36. The disclosure also provides antibodies comprising, or consisting essentially of, or yet further consisting of a heavy chain variable domain sequence that is at least 80%, at least 85%, at least 90%, or at least 95% identical to SEQ ID NO:17 and a light chain variable domain sequence that is at least 80%, at least 85%, at least 90%, or at least 95% identical to SEQ ID NO:37. The disclosure also provides antibodies comprising, or consisting essentially of, or yet further consisting of a heavy chain variable domain sequence that is at least 80%, at least 85%, at least 90%, or at least 95% identical to SEQ ID NO:18 and a light chain variable domain sequence that is at least 80%, at least 85%, at least 90%, or at least 95% identical to SEQ ID NO:38. The disclosure also provides antibodies comprising, or consisting essentially of, or yet further consisting of a heavy chain variable domain sequence that is at least 80%, at least 85%, at least 90%, or at least 95% identical to SEQ ID NO:19 and a light chain variable domain sequence that is at least 80%, at least 85%, at least 90%, or at least 95% identical to SEQ ID NO:39. The disclosure also provides antibodies comprising, or consisting essentially of, or yet further consisting of a heavy chain variable domain sequence that is at least 80%, at least 85%, at least 90%, or at least 95% identical to SEQ ID NO:20 and a light chain variable domain sequence that is at least 80%, at least 85%, at least 90%, or at least 95% identical to SEQ ID NO:40. The disclosure also provides antibodies comprising, or consisting essentially of, or yet further consisting of a heavy chain variable domain sequence that is at least 80%, at least 85%, at least 90%, or at least 95% identical to SEQ ID NO:21 and a light chain variable domain sequence that is at least 80%, at least 85%, at least 90%, or at least 95% identical to SEQ ID NO:41. The disclosure also provides antibodies comprising, or consisting essentially of, or yet further consisting of a heavy chain variable domain sequence that is at least 80%, at least 85%, at least 90%, or at least 95% identical to SEQ ID NO:22 and a light chain variable domain sequence that is at least 80%, at least 85%, at least 90%, or at least 95% identical to SEQ ID NO:42. The disclosure also provides antibodies comprising, or consisting essentially of, or yet further consisting of a heavy chain variable domain sequence that is at least 80%, at least 85%, at least 90%, or at least 95% identical to SEQ ID NO:23 and a light chain variable domain sequence that is at least 80%, at least 85%, at least 90%, or at least 95% identical to SEQ ID NO:43.

In one aspect, provided is an antibody or an antigen binding fragment thereof. The antibody or antigen binding fragment thereof comprises, or consists essentially of, or yet further consists of one or more variable domains as listed in Table 3. In some embodiments, the antibody or antigen binding fragment thereof comprises, or consists essentially of, or yet further consists of a heavy chain variable domain (V_{H}) as listed in Table 3. Additionally or alternatively, the antibody or antigen binding fragment thereof comprises, or consists essentially of, or yet further consists of a light chain variable domain (V_{L}) as listed in Table 3. In some embodiments, the antibody or antigen binding fragment thereof comprises, or consists essentially of, or yet further consists of a V_{H} and a V_{L} as listed in Table 3. In further embodiments, the V_{H} and V_{L} are listed in the same row in Table 3. Exemplary DNA sequences encoding heavy-chain variable domain (VH) and light-chain variable domain (VL) for the anti-SARS-CoV-2 N protein antibodies described herein are depicted in Table 4.

TABLE 3

| | | SEQ | | SEQ |
|---|---|---|---|---|
| | | ID | | ID |
| mAb AA ID | V_{H} Amino Acid Sequence | NO: | V_{L} Amino Acid Sequence | NO: |
| SCT-Pa01 | QVTLKESGPGILQPSQTLSLTCSFDGFSLSTFGM | 4 | QIVLTQSPSIMSASPGEKVTISCSASSSVSYMYW | 24 |
| | GVGWIRQPSGRGLEWLAHIWWDDDKNYKPALKSR | | YQQKPGSSPRPWIYRTSNLASGVPARFSGSGSGT | |
| | LTISKDTSKNQVFLKIANVDTADTATYYCTRMRE | | SYSLTITSMEAEDAATYYCQLYHSYPPTWTFGGG | |
| | DSSDYVYYIMDYWGQGTSVIVAS | | TKLEIK | |
| SCT-Pa02 | QMQLKQSGPGLVQPSQSLSITCTVSGFSLTSYGV | 5 | DIVLTQSPASLAVSLGQRATISCRASESVDYDGD | 25 |
| | HWVRQSPEKGLEWLGVIWSGGGTDYNAAFISRLS | | NYMNWYQQKPGQSPKLLIYATSNLESRIPARFSG | |
| | ISKDNSKSQVFLKMSSLQTDDTAIYYCARGAYYS | | SGSGTEFTLNIHPVEEEDAATYYCLQSYDVPYTF | |
| | LDYWGQGTTLTVSS | | GSGTKLEMK | |
| SCT-Pa03 | QVQLKQSGAELVRPGASVKLSCKASGYTFTDYYI | 6 | DIQMTQSSSYLSVSLGGRVTITCKASDHINNWLA | 26 |
| | NWVKQRPGQGLEWIARIYPGNNYAYYDEKFKGKA | | WYQQKPGNAPRLLISGATSLETGVPSRFSGSGSG | |
| | TLTAEKSSSTAYMQLSSLTSEDSAVYFCAKDDGY | | KDYTLSITSLQTEDVATYYCQQYWSTPPTFGGGT | |
| | RFAYWGQGTLVTVSA | | KLEIK | |
| SCT-Pa04 | EVQLQQSGPELVKPGASVKMSCKASGYSFTSYVM | 7 | SIVMTQTPKFLLVSAGDRITITCKASQSVSNNVA | 27 |
| | HWVKQKPGQGLEWIGYINPYNDGTKYNEKFKGKA | | WYQQKPGQSPTLLISYASNRFTGVPDRFTGSGYG | |
| | TLTSDKSSSTAFMDLSSLTSDDSAVYYCSRDDYS | | TDFTFTISIVQAEDLAVYFCQQDYDSPYTFGGGT | |
| | NPPFAYWGQGTLVTVSA | | KLEIK | |
| SCT-Pa05 | EVQLQQSGAELVKPGASVKLSCTTSGFNIKDYYF | 8 | DIVMTQSPSSLSVWAGEKVTMSCKSSQSLLNSGD | 28 |
| | HWVKQRTEQGLEWIGRIDPEDGETEYAPKFRDKA | | QKNYLTWYQQKPGQSPKLLIYETSTRDYGVPDRF | |
| | TITADTSSNTAYLQLSSLTSEDTAIYYCARSLYY | | TGSGSGADFTLTISSVQAEDLAVYYCLNDLTYPL | |
| | WGQGTTLTVSS | | TFGAGTKLELK | |
| SCT-Pa06 | QVTLKESGPGILQPSQTLSLTCSFSGFSLSTYGM | 9 | ENVLTQSPAIMAASPGEKVTMTCSASSSVSSGNF | 29 |
| | GVGWIRQPSGKGLEWLANIWWNDDKNYNSALKSR | | HWYQQKPGTSPKLWIYRTSNLASGVPARFSGSGS | |
| | LTISKDTSNNQLFLRISSVDTADTATYFCAQIPA | | GTSYSLTISSMEAEDAATYYCQQWSGYPYTFGSG | |
| | YYSNSWFAYWGHGTLVTVSA | | TKLKIK | |
| SCT-Pa07 | EVQLQQSGPELVKPGDSVKMSCKASGYTFTDYYM | 10 | ENVLTQSPAIMSATLGEKVTVSCRARSNVKYMYW | 30 |
| | DWVKQSHGKSLEWIGYIYTNNGDTNYNQKFKGKA | | YQQKSGASPKLLIYYTSNLASGVPARFSGSGSGT | |
| | TLTVDKSSSTAYMELHSLTSEDSAVYYCASLYYS | | SYSLTISSVEAEDAATYYCQQFTSSPFTFGSGTK | |
| | NFFDSWGQGTTLTVSS | | LEIK | |
| SCT-Pa08 | QVQLHQSGAELVKPGASVKLSCKASGYTFTEYSI | 11 | DIVMSQSPSSLAVSVGEKVTMSCKSSQNLLYSSN | 31 |
| | HWVKQRSGQDLEWIGWENPGTGSIKYNEKFKDKA | | QKNFLAWYQQRPGQSPKLLIYWASTRESGVPDRF | |
| | TLTADKSSSTVYMELSRLTSEDSAVYFCARHGGR | | TGSGTGTDFTLTISSVKAEDLAVYYCQQYYRYPF | |
| | WVIQWNFDVWGTGTTVTVSS | | TFGSGTKLEIK | |
| SCT-Pa09 | QVQLQQPGAELVKPGTSVRLSCQASGYTFTSSWI | 12 | QIVLSQSPPILSASPGARVTMTCRASSSVNYMHW | 32 |
| | HWVKQRTGQGLEWIGEISLINGDINYNEKFTSKA | | YQQKPGSSPKPWIYATSNLASGVPVRFSGSGSGT | |
| | TLTVDKSSSTAYMQLSSLTSEDSAVYYCTIIPDG | | SYSLTISRVEAEDAATYYCQQWSSNPPTFGGGTK | |
| | YSLYWYFDVWGAGTTVTVSS | | LEIK | |
| SCT-Pa10 | QVQLQQSGPELVKPGASVKISCKASGYAFSTSWM | 13 | DIQMTQTTSSLSASLGDRVTISCSASQDISDELN | 33 |
| | NWMKQRPGKGLEWIGRIYPGDGETNYNGKFKGKA | | WYQQKPDGTVKLLIYYTSSLHSGVPSRFSGSGSG | |
| | TLTADKSSSTAYMQLSSLTSDDSAVYFCARGANG | | TDYSLTINNLEPEDIATYYCQQYSKLPYTFGGGT | |
| | DYLFYLDYWGQGTTLTVSS | | KLEIR | |
| SCT-Pa11 | EVQLQQSGPELVKPGASVKMSCKASGYTFTSYVI | 14 | DIQMTQSPASLSASVGETVTITCRPSENIYSYLA | 34 |
| | HWVKQKPGQGLEWIGYINPYNDGTEYNEKFKGKA | | WYQQKQGKSPQLLVYNAKTLVEGVPSRFSGSGSG | |
| | TLTSDKSSSTAYMELSSLTSEDSAVYYCSRDDYN | | TQFSLKINSVQPEDCGSYYCLHHFGTPWTFGGGT | |
| | NRFAYWGPGTLVTVSA | | ILEIK | |
| SCT-Pa12 | QVQLQQSGAELARPGASVKLSCKASGYSFTSYGI | 15 | DIQMTQTTSSLSASLGDRVTISCRASQDISNYLN | 35 |
| | SWVKQRTGQGLEWIGDIYPRSGNTDYNEKFKGKA | | WYQQKPDGTVKLLIYYTSRLHSGVPSRFSGSGSG | |
| | TLTADKSSSTANMQLSSLTSEDSAIYFCARSGDG | | TDYSLTISNLEPEDIAPYYCQQYSKLPYTFGSGT | |
| | YLYAMDYWGQGTSVTVSS | | KLEIK | |
| SCT-Pa13 | QVQLKQSGPGLVQPSQSLSITCTVSGFSLSSYAV | 16 | DVVVTQTPLSLPVSFGDQVSISCRSTQSLANSYG | 36 |
| | HWVRQSPGKGLEWLGVIWSGGTTDYNAAFISRLT | | NTYLSWYLHKPGQSPQLLIYGISNRFSGVPDRES | |
| | ISKDNSESQIFFKMNSLQPDDTAIYYCARGAYYS | | GSGSGTDFTLKISTLKPEDLGMYYYLQGTHQYTF | |
| | LDHWGQGTSLTVSS | | GGGTKLEIK | |
| SCT-Pa14 | EVQLQQSGAELVSPGASVKLSCTASGFNIKDDYM | 17 | ENVLTQSPAIMSATLGEKVTMSCRASSNVKYMYW | 37 |
| | HWVKQRPEQGLEWIGRIDPANGNTKYAPKFQDKA | | YQQKSGASPKLWIYYTSNLTSGVPARFSGSGSGT | |
| | TITADTSSNTAYLQLSSLTSEDTAVYYCFYSFYI | | SYSLTISSVEAEDAATYYCQQFTSSPYTFGSGTK | |
| | PYTMDFWGQGTSVTVSS | | LEIK | |
| SCT-Pa15 | QVQVKQSGPGLVQPSQSLSITCTVSGFSSTSYAI | 18 | DIVVTQSPASLAVSLGQRATISCRASESIDYDGD | 38 |
| | HWVRQSPGKGLEWLGVIWSGGTTDYNAAFISRLS | | SYVNWYQQKPGQPPNLLIYAASNLESGIPARFSG | |
| | FSKDNSKSQLFFKMNSLQADDTAMYYCARGNYYG | | GGSGTDFTLTIHPVEEEDAATYYCLQSYETPWTF | |
| | MDYWGQGTSVTVSS | | GGGTKLEIK | |
| SCT-Pa16 | QVQLQQPGTELVKPGASVKLSCKASGYTFTSYWM | 19 | DIVMTQSQKFVSTSVGDRVSVTCKASQNVGTNVV | 39 |
| | HWVKQRPGQGLEWIGSVNPSNGGANYNEKFKNKA | | WYQQKPGQSPKPLIYSASYRYSGVPDRFTGSGSG | |
| | TLTVDKSSSTAYMQLSSLTSEDSAVYYCARRNGF | | TDFTLTISNVQSEDLADYFCHQYNSSPLTFGVGT | |
| | PYYFDYWGQGTTLTVSS | | KLELK | |
| SCT-Pa17 | QVQLQQPGAELVKPGASVKLSCKASGYAFTSSWI | 20 | DIQMTQSSSSFSVSLGDRVTITCKASEDIYNRLA | 40 |
| | NWVKRRPGQGLEWIGNVYPRSSSPNYNEKFKGKA | | WYQQKPGNAPRLLISGATTLETGVPSRFSGSGSG | |
| | TLTVDTSSSTAYMQLSSLTSDDSAVYFCARGYGS | | KDYALSITSLQTEDVSTYYCQEYWSLSPTFGGGT | |
| | YWYFDVWGAGTSVTVSS | | KLEIK | |
| SCT-Pa18 | QVQLQQPGAELVKPGASVKLSCKASGYTFTSYWM | 21 | DIQMTQTTSSLSASLGDRVTISCSASQDISNYLN | 41 |
| | QWVKQRPGQGLEWIGEIAPSDSYTNYNQKFKGKA | | WYQQKPDGTVKLLIYYTSSLHSGVPSRFSGSGSG | |
| | TLTVDTSSSTAYMQLSSLTSEDSAVYYCAGGDYY | | TGYSLTISNLEPEDIATYYCQQYSKLPYTFGGGT | |
| | GYDVKTWFAYWGQGTLVTVSA | | KLEIK | |
| SCT-Pa19 | QVQLQQPGAELVRPGSSVKLSCKASGYTFTNYWM | 22 | DIVMTQSQKFMSTSVGDRVSVTCKASQNVGSNVA | 42 |
| | LWVKQRPMQGLEWIGSIDPSDSETHYNQKFKDKA | | WYQQKSGQSPKALIYSASYRYSGVPDRFTGSGYG | |
| | TLTVDKSSSTAYMRLGSLTSEDSAVYYCARDSYG | | TDFTLTISNVQSEDLAEYFCQQYNNYPLTFGAGT | |
| | WYFDVWGTGTTVTVSS | | KLELK | |
| SCT-Pa20 | QVQLQQPGAELVKPGASVKLSCKASGYTFTDYWI | 23 | DIVLTQSPASLAVTLGQRATISCKASQSINYNGD | 43 |
| | NWVKQRPGQGLEWIGNIYPGSSDTNYNEKFKRKA | | NYMHWFQQKPGQPTKLLIYTASNLESGVPDRFSG | |
| | TLTVDTSSSTAYMQLSSLTSDDSAVYYCARRPLF | | SGSGTDFTLNIHPVEEEDAATYYCQQSKEFPWTF | |
| | HAMDYWGQGTSVTVSS | | GGGTKLEIK | |

TABLE 4

| | | SEQ | Encoded | | SEQ | Encoded |
|---|---|---|---|---|---|---|
| mAb AA ID | V_{H} Encoding DNA | ID | as (SEQ | V_{L} Encoding DNA | ID | as (SEQ |
| | Sequence | NO: | ID NO:) | Sequence | NO: | ID NO:) |
| SCT-Pa01 | CAGGTTACTCTGAAAGAGTCTG | 148 | 4 | CAAATTGTTCTCACCCAGTC | 168 | 24 |
| | GCCCTGGGATATTGCAGCCCTC | | | TCCATCAATCATGTCTGCAT | | |
| | CCAGACCCTCAGTCTGACTTGT | | | CTCCAGGGGAGAAGGTCACC | | |
| | TCTTTCGATGGGTTTTCACTGA | | | ATATCCTGCAGTGCCAGCTC | | |
| | GCACTTTTGGTATGGGTGTCGG | | | AAGTGTAAGTTACATGTACT | | |
| | CTGGATTCGTCAGCCTTCAGGG | | | GGTACCAGCAGAAGCCAGGA | | |
| | AGGGGTCTGGAGTGGCTGGCAC | | | TCCTCCCCCAGACCCTGGAT | | |
| | ACATTTGGTGGGATGATGATAA | | | TTATCGCACATCCAACCTGG | | |
| | GAACTATAAACCAGCCCTGAAG | | | CTTCTGGAGTCCCTGCTCGC | | |
| | AGCCGGCTCACAATCTCCAAGG | | | TTCAGTGGCAGTGGGTCTGG | | |
| | ATACCTCCAAAAACCAGGTATT | | | GACCTCTTACTCTCTCACAA | | |
| | CCTCAAGATCGCCAATGTGGAC | | | TCACCAGCATGGAGGCTGAA | | |
| | ACTGCAGATACTGCCACATACT | | | GATGCTGCCACTTATTACTG | | |
| | ACTGTACTCGAATGAGGGAGGA | | | CCAGCTGTATCATAGTTACC | | |
| | CAGCTCGGACTACGTTTATTAT | | | CACCCACGTGGACGTTCGGT | | |
| | ATTATGGACTATTGGGGTCAAG | | | GGAGGCACCAAGCTGGAAAT | | |
| | GAACCTCAGTCATCGTCGCCTC | | | CAAA | | |
| | A | | | | | |
| SCT-Pa02 | CAGATGCAGCTGAAGCAGTCAG | 149 | 5 | GACATTGTGCTGACCCAATC | 169 | 25 |
| | GACCTGGCCTAGTGCAGCCCTC | | | TCCAGCTTCATTGGCTGTGT | | |
| | ACAGAGCCTGTCCATCACCTGC | | | CTCTAGGGCAGAGGGCCACC | | |
| | ACAGTCTCTGGTTTCTCATTAA | | | ATCTCCTGCAGAGCCAGCGA | | |
| | CTAGCTATGGTGTTCACTGGGT | | | AAGTGTTGATTATGATGGCG | | |
| | TCGCCAGTCTCCAGAAAAGGGT | | | ATAATTATATGAACTGGTAC | | |
| | CTGGAGTGGCTGGGAGTGATAT | | | CAACAGAAACCAGGACAGTC | | |
| | GGAGTGGTGGAGGTACAGACTA | | | ACCGAAACTCCTCATCTACG | | |
| | TAATGCAGCTTTCATATCCAGA | | | CTACATCCAATCTAGAATCT | | |
| | CTGAGCATCAGCAAGGACAATT | | | AGGATCCCTGCCAGGTTTAG | | |
| | CCAAGAGCCAAGTTTTCCTTAA | | | TGGCAGTGGGTCTGGGACAG | | |
| | AATGAGCAGTCTGCAAACTGAT | | | AATTCACCCTCAACATTCAT | | |
| | GACACAGCCATATATTACTGTG | | | CCTGTGGAGGAGGAAGATGC | | |
| | CCAGAGGGGCCTACTATAGTCT | | | TGCAACCTATTACTGTCTGC | | |
| | TGACTACTGGGGCCAAGGCACC | | | AAAGTTATGACGTTCCGTAT | | |
| | ACTCTCACAGTCTCCTCA | | | ACGTTCGGATCGGGGACCAA | | |
| | | | | GCTGGAAATGAAA | | |
| SCT-Pa03 | CAGGTCCAGCTGAAGCAGTCTG | 150 | 6 | GACATCCAGATGACACAATC | 170 | 26 |
| | GGGCTGAGCTGGTGAGGCCTGG | | | TTCATCCTACTTGTCTGTAT | | |
| | GGCTTCAGTGAAGCTGTCCTGC | | | CTCTAGGAGGCAGAGTCACC | | |
| | AAGGCTTCTGGCTACACTTTCA | | | ATTACTTGCAAGGCAAGTGA | | |
| | CTGACTACTATATAAACTGGGT | | | CCACATTAATAATTGGTTAG | | |
| | GAAGCAGAGGCCTGGACAGGGA | | | CCTGGTATCAGCAGAAACCA | | |
| | CTTGAGTGGATTGCAAGGATTT | | | GGAAATGCTCCTAGGCTCTT | | |
| | ATCCTGGAAATAATTATGCTTA | | | AATATCTGGTGCAACCAGTT | | |
| | CTACGATGAGAAGTTCAAGGGC | | | TGGAAACTGGGGTTCCTTCA | | |
| | AAGGCCACACTGACTGCAGAAA | | | AGATTCAGTGGCAGTGGATC | | |
| | AATCCTCCAGCACTGCCTACAT | | | TGGAAAGGATTACACTCTCA | | |
| | GCAGCTCAGCAGCCTGACATCT | | | GCATTACCAGTCTTCAGACT | | |
| | GAGGACTCTGCTGTCTATTTCT | | | GAAGATGTTGCTACTTATTA | | |
| | GTGCAAAGGATGATGGTTACCG | | | CTGTCAACAGTATTGGAGTA | | |
| | GTTTGCTTACTGGGGCCAAGGG | | | CTCCTCCGACGTTCGGTGGA | | |
| | ACTCTGGTCACTGTCTCTGCA | | | GGCACCAAGCTGGAAATCAA | | |
| | | | | A | | |
| SCT-Pa04 | GAGGTCCAGCTGCAGCAGTCTG | 151 | 7 | AGTATTGTGATGACCCAGAC | 171 | 27 |
| | GACCTGAGCTGGTAAAGCCAGG | | | TCCCAAATTCCTGCTTGTAT | | |
| | GGCTTCAGTGAAGATGTCCTGC | | | CAGCAGGAGACAGGATTACC | | |
| | AAGGCTTCTGGATACTCATTCA | | | ATAACCTGCAAGGCCAGTCA | | |
| | CTAGTTATGTTATGCACTGGGT | | | GAGTGTGAGTAATAATGTAG | | |
| | GAAGCAGAAGCCTGGGCAGGGC | | | CTTGGTACCAACAGAAGCCA | | |
| | CTTGAATGGATTGGATATATTA | | | GGGCAGTCTCCTACACTGCT | | |
| | ATCCTTACAATGATGGTACTAA | | | GATATCCTATGCATCCAATC | | |
| | GTACAATGAGAAGTTCAAAGGC | | | GCTTCACTGGAGTCCCTGAT | | |
| | AAGGCCACACTGACTTCAGACA | | | CGCTTCACTGGCAGTGGATA | | |
| | AATCCTCCAGCACAGCCTTCAT | | | TGGGACGGATTTCACTTTCA | | |
| | GGACCTCAGCAGCCTGACCTCT | | | CCATCAGCATTGTGCAGGCT | | |
| | GATGACTCTGCGGTCTATTACT | | | GAAGACCTGGCAGTTTATTT | | |
| | GTTCAAGAGACGACTATAGTAA | | | CTGTCAGCAGGATTATGACT | | |
| | CCCCCCTTTTGCTTACTGGGGC | | | CTCCGTACACGTTCGGAGGG | | |
| | CAAGGGACTTTGGTCACTGTCT | | | GGGACCAAGCTGGAAATAAA | | |
| | CTGCA | | | A | | |
| SCT-Pa05 | GAGGTTCAGCTGCAGCAGTCTG | 152 | 8 | GACATTGTGATGACACAGTC | 172 | 28 |
| | GGGCAGAGCTTGTGAAGCCAGG | | | TCCATCCTCCCTGAGTGTGT | | |
| | GGCCTCAGTCAAGTTGTCCTGC | | | GGGCAGGAGAGAAGGTCACT | | |
| | ACAACTTCTGGCTTCAACATTA | | | ATGAGCTGCAAGTCCAGTCA | | |
| | AAGACTACTATTTTCACTGGGT | | | GAGTCTGTTAAACAGTGGAG | | |
| | GAAACAGAGGACTGAGCAGGGC | | | ATCAGAAGAACTACTTGACC | | |
| | CTGGAGTGGATTGGAAGGATTG | | | TGGTACCAGCAGAAACCAGG | | |
| | ATCCTGAGGATGGTGAAACTGA | | | ACAGTCTCCTAAACTGTTGA | | |
| | ATATGCCCCGAAATTCCGGGAC | | | TCTACGAGACATCCACTAGG | | |
| | AAGGCCACTATAACAGCAGACA | | | GATTATGGGGTCCCTGATCG | | |
| | CATCCTCCAACACAGCCTACCT | | | CTTCACAGGCAGTGGATCTG | | |
| | GCAGCTCAGCAGCCTGACATCT | | | GAGCCGATTTCACTCTTACC | | |
| | GAGGACACTGCCATCTATTACT | | | ATCAGCAGTGTGCAGGCTGA | | |
| | GTGCTAGATCCCTCTACTACTG | | | AGACCTGGCAGTTTATTACT | | |
| | GGGCCAAGGCACCACTCTCACA | | | GTCTGAATGATCTTACTTAT | | |
| | GTCTCCTCA | | | CCGCTCACATTCGGTGCTGG | | |
| | | | | GACCAAGCTGGAACTGAAA | | |
| SCT-Pa06 | CAGGTCACTCTGAAAGAGTCTG | 153 | 9 | GAAAATGTGCTGACCCAGTC | 173 | 29 |
| | GCCCTGGGATATTGCAGCCCTC | | | TCCAGCAATCATGGCTGCAT | | |
| | CCAGACCCTCAGTCTGACTTGT | | | CTCCAGGGGAGAAGGTCACC | | |
| | TCTTTCTCTGGGTTTTCACTGA | | | ATGACCTGCAGTGCCAGCTC | | |
| | GCACTTATGGTATGGGTGTAGG | | | AAGTGTAAGTTCTGGTAACT | | |
| | TTGGATTCGTCAGCCTTCAGGG | | | TTCACTGGTACCAGCAGAAG | | |
| | AAGGGTCTGGAGTGGCTGGCCA | | | CCAGGCACTTCTCCCAAACT | | |
| | ACATTTGGTGGAATGATGATAA | | | CTGGATTTATAGGACATCCA | | |
| | GAACTATAATTCAGCCCTGAAG | | | ACCTGGCTTCTGGAGTCCCC | | |
| | AGCCGGCTCACAATCTCCAAGG | | | GCTCGCTTCAGTGGCAGTGG | | |
| | ATACCTCCAACAACCAACTATT | | | GTCTGGGACCTCTTACTCTC | | |
| | CCTCAGGATCTCCAGTGTGGAC | | | TTACAATCAGCAGCATGGAG | | |
| | ACTGCAGATACTGCCACATACT | | | GCCGAGGATGCTGCCACTTA | | |
| | TCTGTGCTCAAATACCTGCCTA | | | TTACTGCCAGCAGTGGAGTG | | |
| | CTATAGTAACTCCTGGTTTGCT | | | GTTACCCGTATACGTTCGGA | | |
| | TACTGGGGCCACGGGACTCTGG | | | TCGGGGACCAAACTGAAAAT | | |
| | TCACTGTCTCTGCA | | | AAAA | | |
| SCT-Pa07 | GAAGTCCAGCTACAACAGTCTG | 154 | 10 | GAAAATGTGCTCACCCAGTC | 174 | 30 |
| | GACCTGAGCTGGTGAAGCCTGG | | | TCCAGCAATCATGTCTGCAA | | |
| | GGATTCAGTGAAGATGTCCTGC | | | CTCTAGGAGAGAAAGTCACC | | |
| | AAGGCTTCTGGCTACACATTCA | | | GTGAGCTGCAGGGCCCGCTC | | |
| | CTGACTACTACATGGACTGGGT | | | AAATGTAAAATACATGTACT | | |
| | GAAGCAGAGCCATGGAAAGAGC | | | GGTACCAGCAGAAGTCAGGT | | |
| | CTTGAGTGGATTGGATATATTT | | | GCCTCCCCCAAACTATTGAT | | |
| | ATACTAACAATGGTGATACTAA | | | TTATTACACATCCAACCTGG | | |
| | CTACAATCAGAAGTTCAAGGGC | | | CTTCTGGAGTCCCAGCTCGC | | |
| | AAGGCCACATTGACTGTAGACA | | | TTCAGTGGCAGTGGGTCTGG | | |
| | AGTCCTCCAGTACAGCCTACAT | | | GACCTCTTATTCTCTCACAA | | |
| | GGAGCTCCACAGCCTGACATCT | | | TCAGCAGCGTGGAGGCTGAA | | |
| | GAGGACTCTGCAGTCTATTACT | | | GATGCTGCCACTTATTACTG | | |
| | GTGCAAGTCTCTACTATAGTAA | | | CCAGCAGTTTACTAGCTCCC | | |
| | CTTTTTTGACTCCTGGGGCCAA | | | CATTTACGTTCGGATCGGGG | | |
| | GGCACCACTCTCACAGTCTCCT | | | ACCAAGCTGGAAATAAAA | | |
| | CA | | | | | |
| SCT-Pa08 | CAGGTCCAGCTGCACCAGTCTG | 155 | 11 | GACATTGTGATGTCACAGTC | 175 | 31 |
| | GAGCTGAGCTGGTGAAACCCGG | | | TCCATCCTCCCTAGCTGTGT | | |
| | GGCATCAGTGAAGCTGTCCTGC | | | CAGTTGGAGAGAAGGTTACT | | |
| | AAGGCTTCTGGCTACACCTTCA | | | ATGAGCTGCAAGTCCAGTCA | | |
| | CTGAGTATAGTATTCACTGGGT | | | GAACCTTTTATATAGTAGCA | | |
| | AAAGCAGAGGTCTGGACAGGAT | | | ATCAAAAGAACTTCTTGGCC | | |
| | CTTGAGTGGATTGGGTGGTTTA | | | TGGTACCAACAGAGACCAGG | | |
| | ACCCTGGAACAGGTAGTATAAA | | | GCAGTCTCCTAAACTGCTGA | | |
| | GTACAATGAGAAATTCAAGGAC | | | TTTACTGGGCATCCACTAGG | | |
| | AAGGCCACATTGACTGCGGACA | | | GAATCTGGGGTCCCTGATCG | | |
| | AATCCTCCAGCACAGTCTATAT | | | CTTCACAGGCAGTGGAACTG | | |
| | GGAGCTTAGTAGATTGACATCT | | | GGACAGATTTCACTCTCACC | | |
| | GAAGACTCTGCGGTCTATTTCT | | | ATCAGCAGTGTGAAGGCTGA | | |
| | GTGCAAGACACGGAGGCAGATG | | | AGACCTGGCAGTTTATTACT | | |
| | GGTAATACAGTGGAACTTCGAT | | | GTCAGCAATATTATAGATAT | | |
| | GTCTGGGGCACAGGGACCACGG | | | CCATTCACGTTCGGCTCGGG | | |
| | TCACCGTCTCCTCA | | | GACAAAGTTGGAAATAAAA | | |
| SCT-Pa09 | CAGGTCCAATTGCAGCAGCCTG | 156 | 12 | CAAATTGTTCTCTCCCAGTC | 176 | 32 |
| | GGGCTGAGCTTGTGAAGCCTGG | | | TCCACCAATCCTGTCTGCAT | | |
| | GACTTCAGTGAGGTTGTCCTGC | | | CTCCAGGGGCGAGGGTCACA | | |
| | CAGGCTTCTGGCTACACCTTCA | | | ATGACTTGCAGGGCCAGCTC | | |
| | CCAGCTCCTGGATACACTGGGT | | | AAGTGTGAATTACATGCATT | | |
| | GAAGCAGAGGACTGGACAAGGC | | | GGTACCAGCAGAAGCCAGGA | | |
| | CTTGAGTGGATTGGGGAGATTA | | | TCCTCCCCCAAACCCTGGAT | | |
| | GTCTTACCAATGGTGATATTAA | | | TTATGCCACATCCAACCTGG | | |
| | CTACAATGAGAAGTTCACGAGC | | | CTTCTGGAGTCCCTGTTCGC | | |
| | AAGGCCACACTGACTGTTGACA | | | TTCAGTGGCAGTGGGTCTGG | | |
| | AATCCTCCAGCACCGCCTACAT | | | GACCTCTTACTCTCTCACAA | | |
| | GCAACTTAGCAGCCTGACATCT | | | TCAGCAGAGTGGAGGCTGAA | | |
| | GAGGACTCTGCGGTCTATTACT | | | GATGCTGCCACTTATTACTG | | |
| | GTACAATAATCCCTGATGGTTA | | | CCAGCAGTGGAGTAGTAACC | | |
| | CTCCCTCTACTGGTACTTCGAT | | | CACCCACGTTCGGAGGGGGG | | |
| | GTCTGGGGCGCAGGGACCACGG | | | ACCAAGCTGGAAATAAAA | | |
| | TCACCGTCTCCTCA | | | | | |
| SCT-Pa10 | CAGGTTCAACTGCAGCAGTCTG | 157 | 13 | GATATCCAGATGACACAGAC | 177 | 33 |
| | GACCTGAGCTGGTGAAGCCTGG | | | TACATCCTCCCTGTCTGCCT | | |
| | GGCCTCAGTGAAGATTTCCTGC | | | CTCTGGGAGACAGAGTCACC | | |
| | AAGGCTTCTGGCTACGCATTCA | | | ATCAGTTGCAGTGCAAGTCA | | |
| | GTACCTCCTGGATGAACTGGAT | | | GGACATTAGCGATTTTTTAA | | |
| | GAAGCAGAGGCCTGGAAAGGGT | | | ACTGGTATCAGCAGAAACCA | | |
| | CTTGAGTGGATTGGACGGATTT | | | GATGGAACTGTTAAACTCCT | | |
| | ATCCTGGAGATGGAGAAACTAA | | | GATCTATTACACATCAAGTT | | |
| | TTATAATGGGAAATTCAAGGGC | | | TACACTCAGGAGTCCCATCA | | |
| | AAGGCCACACTGACTGCAGACA | | | AGGTTCAGTGGCAGTGGGTC | | |
| | AATCCTCCAGCACAGCCTACAT | | | TGGGACAGATTATTCTCTCA | | |
| | GCAACTCAGCAGCCTGACATCT | | | CCATCAACAACCTGGAACCT | | |
| | GACGACTCTGCGGTCTACTTCT | | | GAAGATATTGCCACTTATTA | | |
| | GTGCAAGAGGGGCCAATGGTGA | | | TTGTCAGCAGTATAGTAAGC | | |
| | CTACTTGTTCTACCTTGACTAC | | | TTCCGTACACGTTCGGAGGG | | |
| | TGGGGCCAAGGCACCACTCTCA | | | GGGACCAAGCTGGAAATAAG | | |
| | CAGTCTCCTCA | | | A | | |
| SCT-Pa11 | GAGGTCCAGCTGCAGCAGTCTG | 158 | 14 | GACATCCAGATGACTCAGTC | 178 | 34 |
| | GACCTGAGCTGGTAAAGCCTGG | | | TCCAGCCTCCCTATCTGCAT | | |
| | GGCTTCAGTGAAGATGTCCTGC | | | CTGTGGGAGAAACTGTCACC | | |
| | AAGGCTTCTGGATACACATTCA | | | ATCACATGTCGACCAAGTGA | | |
| | CTAGCTATGTTATACACTGGGT | | | GAATATTTACAGTTATTTAG | | |
| | GAAGCAGAAGCCTGGGCAGGGC | | | CATGGTATCAGCAGAAACAG | | |
| | CTTGAGTGGATTGGATATATTA | | | GGAAAATCTCCTCAGCTCCT | | |
| | ATCCTTACAATGATGGTACTGA | | | GGTCTATAATGCAAAAACCT | | |
| | GTACAATGAGAAGTTCAAGGGC | | | TAGTAGAAGGTGTGCCATCC | | |
| | AAGGCCACACTGACTTCAGACA | | | AGGTTCAGTGGCAGTGGATC | | |
| | AATCCTCCAGTACAGCCTACAT | | | AGGCACACAGTTTTCTCTGA | | |
| | GGAGCTCAGCAGCCTGACCTCT | | | AGATCAACAGTGTGCAGCCT | | |
| | GAGGACTCTGCGGTCTATTACT | | | GAAGATTGTGGGAGTTATTA | | |
| | GTTCAAGAGATGACTATAATAA | | | CTGTCTACATCATTTTGGTA | | |
| | CCGGTTTGCTTACTGGGGCCCA | | | CTCCCTGGACGTTCGGCGGA | | |
| | GGGACTCTGGTCACTGTCTCTG | | | GGCACCATCCTGGAAATCAA | | |
| | CA | | | A | | |
| SCT-Pa12 | CAGGTTCAGCTGCAGCAGTCTG | 159 | 15 | GATATCCAGATGACACAGAC | 179 | 35 |
| | GAGCTGAGCTGGCGAGGCCTGG | | | TACATCCTCCCTGTCTGCCT | | |
| | GGCTTCAGTGAAGCTGTCCTGC | | | CTCTGGGAGACAGAGTCACC | | |
| | AAGGCTTCTGGCTACAGTTTCA | | | ATCAGTTGCAGGGCAAGTCA | | |
| | CAAGCTATGGTATAAGTTGGGT | | | GGATATTAGCAATTATTTAA | | |
| | GAAGCAGAGAACTGGACAGGGC | | | ACTGGTATCAGCAGAAACCA | | |
| | CTTGAGTGGATTGGAGACATTT | | | GATGGAACTGTTAAACTCCT | | |
| | ATCCTAGAAGTGGTAATACTGA | | | GATCTACTACACATCAAGAT | | |
| | CTACAATGAGAAGTTCAAGGGC | | | TACACTCAGGAGTCCCATCA | | |
| | AAGGCCACACTGACTGCAGACA | | | AGGTTCAGTGGCAGTGGGTC | | |
| | AATCCTCCAGCACAGCCAACAT | | | TGGGACAGATTATTCTCTCA | | |
| | GCAGCTCAGCAGCCTGACATCT | | | CCATCAGCAACCTGGAACCT | | |
| | GAGGACTCTGCAATCTATTTCT | | | GAAGATATTGCCCCTTACTA | | |
| | GTGCAAGATCGGGAGATGGTTA | | | TTGTCAGCAGTATAGTAAGC | | |
| | CCTCTATGCTATGGACTACTGG | | | TTCCGTATACGTTCGGATCG | | |
| | GGTCAAGGAACCTCAGTCACCG | | | GGGACCAAGCTGGAAATAAA | | |
| | TCTCCTCA | | | A | | |
| SCT-Pa13 | CAGGTGCAACTGAAGCAGTCAG | 160 | 16 | GATGTTGTGGTGACTCAAAC | 180 | 36 |
| | GACCTGGCCTAGTGCAGCCCTC | | | TCCACTCTCCCTGCCTGTCA | | |
| | ACAGAGCCTGTCCATCACCTGC | | | GCTTTGGAGATCAAGTTTCT | | |
| | ACAGTCTCTGGTTTCTCATTAA | | | ATCTCTTGCAGGTCTACTCA | | |
| | GTAGCTATGCTGTACACTGGGT | | | GAGTCTTGCAAACAGTTATG | | |
| | TCGCCAGTCTCCAGGAAAGGGT | | | GGAACACCTATTTGTCTTGG | | |
| | CTGGAGTGGCTGGGAGTGATAT | | | TACCTACACAAGCCTGGCCA | | |
| | GGAGTGGTGGAACCACAGACTA | | | GTCTCCACAGCTCCTCATCT | | |
| | TAATGCAGCTTTCATATCCAGA | | | ATGGGATTTCCAACAGATTT | | |
| | CTGACCATCAGCAAGGACAATT | | | TCTGGGGTGCCAGACAGGTT | | |
| | CCGAGAGCCAAATTTTCTTTAA | | | CAGTGGCAGTGGTTCAGGGA | | |
| | AATGAACAGTCTTCAACCTGAT | | | CAGATTTCACACTCAAGATC | | |
| | GACACAGCCATATATTATTGTG | | | AGCACATTAAAGCCTGAGGA | | |
| | CCAGAGGGGCCTACTATAGTCT | | | CTTGGGAATGTATTACTACT | | |
| | TGACCACTGGGGCCAAGGCACC | | | TACAAGGTACACATCAGTAC | | |
| | TCTCTCACAGTCTCCTCA | | | ACATTCGGAGGGGGGACCAA | | |
| | | | | GCTGGAAATAAAA | | |
| SCT-Pa14 | GAGGTTCAGCTGCAGCAGTCTG | 161 | 17 | GAAAATGTGCTCACCCAGTC | 181 | 37 |
| | GGGCTGAGCTTGTGAGTCCAGG | | | TCCAGCAATCATGTCTGCAA | | |
| | GGCCTCAGTCAAGTTGTCCTGC | | | CTCTTGGGGAGAAGGTCACC | | |
| | ACAGCTTCTGGCTTTAACATTA | | | ATGAGTTGCAGGGCCAGCTC | | |
| | AAGACGACTATATGCACTGGGT | | | AAATGTAAAGTACATGTACT | | |
| | GAAGCAGAGGCCTGAACAGGGC | | | GGTACCAACAGAAGTCAGGT | | |
| | CTGGAGTGGATTGGAAGGATTG | | | GCCTCCCCCAAACTCTGGAT | | |
| | ATCCTGCGAATGGTAATACTAA | | | TTATTACACATCCAACCTGA | | |
| | ATATGCCCCGAAGTTCCAGGAC | | | CTTCTGGAGTCCCAGCTCGC | | |
| | AAGGCCACTATAACTGCAGACA | | | TTCAGTGGCAGTGGGTCTGG | | |
| | CATCCTCCAACACAGCCTACCT | | | GACCTCTTATTCTCTCACAA | | |
| | GCAGCTCAGCAGCCTGACATCT | | | TCAGCAGCGTGGAGGCTGAG | | |
| | GAGGACACTGCCGTCTATTACT | | | GATGCTGCCACTTATTACTG | | |
| | GTTTCTATAGTTTCTATATTCC | | | CCAGCAGTTTACTAGTTCCC | | |
| | TTATACTATGGACTTCTGGGGT | | | CATATACGTTCGGATCGGGG | | |
| | CAAGGAACCTCAGTCACCGTCT | | | ACCAAACTGGAAATAAAA | | |
| | CCTCA | | | | | |
| SCT-Pa15 | CAGGTGCAGGTGAAGCAGTCAG | 162 | 18 | GACATTGTGGTGACCCAATC | 182 | 38 |
| | GACCTGGCCTGGTGCAGCCCTC | | | TCCAGCTTCTTTGGCTGTGT | | |
| | ACAGAGCCTGTCCATCACCTGC | | | CTCTAGGGCAGAGGGCCACC | | |
| | ACAGTCTCTGGTTTCTCATCAA | | | ATCTCCTGCAGAGCCAGCGA | | |
| | CTAGTTATGCTATACACTGGGT | | | AAGTATTGACTATGATGGCG | | |
| | TCGCCAGTCTCCAGGAAAGGGT | | | ATAGTTATGTGAACTGGTAC | | |
| | CTGGAGTGGCTGGGAGTGATAT | | | CAACAGAAACCAGGACAGCC | | |
| | GGAGTGGTGGAACCACAGACTA | | | ACCGAATCTCCTCATCTATG | | |
| | TAATGCAGCTTTCATATCCAGA | | | CTGCATCCAATCTAGAATCT | | |
| | CTGAGCTTCAGCAAGGACAATT | | | GGCATCCCTGCCAGGTTTAG | | |
| | CCAAGAGCCAACTTTTCTTTAA | | | TGGCGGTGGGTCGGGGACAG | | |
| | AATGAACAGTCTGCAAGCTGAT | | | ACTTCACCCTCACCATTCAT | | |
| | GACACAGCCATGTATTACTGTG | | | CCTGTGGAGGAGGAGGATGC | | |
| | CCAGAGGTAATTACTATGGTAT | | | TGCAACCTATTACTGTCTGC | | |
| | GGACTACTGGGGTCAAGGAACC | | | AAAGTTATGAGACTCCGTGG | | |
| | TCGGTCACCGTCTCCTCA | | | ACGTTCGGTGGAGGCACCAA | | |
| | | | | GCTGGAAATCAAA | | |
| SCT-Pa16 | CAGGTCCAACTGCAGCAGCCTG | 163 | 19 | GACATTGTGATGACCCAGTC | 183 | 39 |
| | GGACTGAACTGGTGAAGCCTGG | | | TCAAAAATTCGTGTCCACAT | | |
| | GGCTTCAGTGAAGCTGTCCTGC | | | CAGTAGGAGACAGGGTCAGC | | |
| | AAGGCTTCTGGCTACACCTTCA | | | GTCACCTGCAAGGCCAGTCA | | |
| | CCAGCTACTGGATGCACTGGGT | | | GAATGTGGGTACTAATGTAG | | |
| | GAAGCAGAGGCCTGGACAAGGC | | | TCTGGTATCAACAGAAACCA | | |
| | CTTGAGTGGATTGGAAGTGTTA | | | GGGCAATCTCCTAAACCACT | | |
| | ATCCTAGCAATGGTGGTGCTAA | | | GATTTACTCGGCATCCTACC | | |
| | CTACAATGAGAAGTTCAAGAAC | | | GGTACAGTGGAGTCCCTGAT | | |
| | AAGGCCACACTGACTGTAGACA | | | CGCTTCACAGGCAGTGGATC | | |
| | AATCCTCCAGCACAGCCTACAT | | | TGGGACAGATTTCACTCTCA | | |
| | GCAACTCAGCAGCCTGACATCT | | | CCATCAGCAATGTGCAGTCT | | |
| | GAGGACTCTGCGGTCTATTATT | | | GAAGACTTGGCAGACTATTT | | |
| | GTGCAAGAAGGAATGGTTTCCC | | | CTGTCACCAATATAACAGTT | | |
| | GTACTACTTTGACTACTGGGGC | | | CTCCTCTCACGTTCGGTGTT | | |
| | CAAGGCACCACTCTCACAGTCT | | | GGGACCAAGCTGGAGCTGAA | | |
| | CCTCA | | | A | | |
| SCT-Pa17 | CAGGTCCAACTGCAGCAGCCTG | 164 | 20 | GACATCCAGATGACACAATC | 184 | 40 |
| | GTGCTGAGCTTGTGAAGCCTGG | | | TTCATCCTCCTTTTCTGTAT | | |
| | GGCCTCAGTGAAGCTGTCCTGC | | | CTCTAGGAGACAGAGTCACC | | |
| | AAGGCTTCTGGCTACGCTTTCA | | | ATTACTTGCAAGGCAAGTGA | | |
| | CCAGCTCCTGGATAAACTGGGT | | | GGACATATATAATCGGTTAG | | |
| | GAAGCGGAGGCCTGGACAAGGC | | | CCTGGTATCAGCAGAAACCA | | |
| | CTTGAGTGGATTGGAAATGTTT | | | GGAAATGCTCCTAGGCTCTT | | |
| | ATCCTCGTAGTAGTAGTCCTAA | | | AATATCTGGTGCAACCACTT | | |
| | CTACAATGAGAAGTTCAAGGGC | | | TGGAAACTGGGGTTCCTTCA | | |
| | AAGGCCACACTGACTGTTGACA | | | AGATTCAGTGGCAGTGGCTC | | |
| | CATCCTCCAGCACAGCCTACAT | | | TGGAAAGGATTACGCTCTCA | | |
| | GCAGCTCAGCAGCCTGACATCT | | | GCATTACAAGTCTTCAGACT | | |
| | GACGACTCTGCGGTCTATTTTT | | | GAAGATGTTTCTACTTATTA | | |
| | GTGCAAGAGGATATGGTAGTTA | | | CTGTCAAGAGTATTGGAGTC | | |
| | CTGGTACTTCGATGTCTGGGGC | | | TTTCTCCGACGTTCGGTGGA | | |
| | GCAGGGACCTCGGTCACCGTCT | | | GGCACCAAGCTGGAAATCAA | | |
| | CCTCA | | | A | | |
| SCT-Pa18 | CAGGTCCAACTGCAGCAGCCTG | 165 | 21 | GATATCCAGATGACACAGAC | 185 | 41 |
| | GGGCTGAGCTTGTGAAGCCTGG | | | TACGTCCTCCCTGTCTGCCT | | |
| | GGCTTCAGTGAAGCTGTCCTGC | | | CTCTGGGAGACAGAGTCACC | | |
| | AAGGCTTCTGGCTACACCTTCA | | | ATCAGTTGCAGTGCAAGTCA | | |
| | CCAGCTACTGGATGCAGTGGGT | | | GGACATTAGCAATTATTTAA | | |
| | AAAACAGAGGCCTGGACAGGGC | | | ACTGGTATCAGCAGAAACCA | | |
| | CTTGAGTGGATCGGAGAGATTG | | | GATGGAACTGTTAAACTCCT | | |
| | CTCCTTCTGATAGCTATACTAA | | | GATCTATTACACATCAAGTT | | |
| | CTACAATCAAAAGTTCAAGGGC | | | TACACTCAGGAGTCCCATCA | | |
| | AAGGCCACATTGACTGTAGACA | | | AGGTTCAGTGGCAGTGGGTC | | |
| | CATCCTCCAGCACAGCCTACAT | | | TGGGACAGGTTATTCTCTCA | | |
| | GCAGCTCAGCAGCCTGACATCT | | | CCATCAGCAACCTGGAACCT | | |
| | GAGGACTCTGCGGTCTATTACT | | | GAAGATATTGCCACTTACTA | | |
| | GTGCAGGAGGGGACTACTATGG | | | TTGTCAGCAGTATAGTAAGC | | |
| | TTACGACGTAAAGACTTGGTTT | | | TTCCGTACACGTTCGGAGGG | | |
| | GCTTACTGGGGCCAAGGGACTC | | | GGGACCAAGCTGGAAATAAA | | |
| | TGGTCACTGTCTCTGCA | | | A | | |
| SCT-Pa19 | CAGGTCCAACTGCAGCAGCCTG | 166 | 22 | GACATTGTGATGACCCAGTC | 186 | 42 |
| | GGGCTGAGCTGGTGAGGCCTGG | | | TCAAAAATTCATGTCCACAT | | |
| | GTCTTCAGTGAAACTGTCCTGC | | | CAGTAGGAGACAGGGTCAGC | | |
| | AAGGCTTCTGGCTACACCTTCA | | | GTCACCTGCAAGGCCAGTCA | | |
| | CCAACTACTGGATGCTTTGGGT | | | GAATGTGGGTTCTAATGTAG | | |
| | GAAGCAGAGGCCTATGCAAGGC | | | CCTGGTATCAACAGAAATCA | | |
| | CTTGAATGGATTGGTAGCATTG | | | GGGCAATCTCCTAAAGCACT | | |
| | ACCCTTCTGATAGTGAAACTCA | | | GATTTACTCGGCATCCTACC | | |
| | CTACAATCAAAAGTTCAAGGAC | | | GGTACAGTGGAGTCCCTGAT | | |
| | AAGGCCACATTGACTGTAGACA | | | CGCTTCACAGGCAGTGGATA | | |
| | AATCCTCCAGCACAGCCTACAT | | | TGGGACAGATTTCACTCTCA | | |
| | GCGGCTCGGCAGCCTGACATCT | | | CCATCAGCAATGTGCAGTCT | | |
| | GAGGACTCTGCGGTCTATTACT | | | GAAGATTTGGCAGAGTATTT | | |
| | GTGCAAGAGATTCCTACGGTTG | | | CTGTCAGCAATATAACAACT | | |
| | GTACTTCGATGTCTGGGGCACA | | | ATCCGCTCACGTTCGGTGCT | | |
| | GGGACCACGGTCACCGTCTCCT | | | GGGACCAAGCTGGAGCTGAA | | |
| | CA | | | A | | |
| SCT-Pa20 | CAGGTCCAATTGCAGCAGCCTG | 167 | 23 | GACATTGTGCTGACCCAATC | 187 | 43 |
| | GTGCTGAGCTTGTGAAGCCTGG | | | TCCAGCTTCTTTGGCTGTGA | | |
| | GGCCTCAGTGAAGCTGTCCTGC | | | CTCTAGGACAGAGGGCCACC | | |
| | AAGGCTTCTGGCTACACTTTCA | | | ATATCCTGCAAGGCCAGCCA | | |
| | CCGACTACTGGATAAACTGGGT | | | GAGTATCAATTATAACGGTG | | |
| | GAAGCAGAGGCCTGGACAAGGC | | | ATAATTATATGCACTGGTTC | | |
| | CTTGAGTGGATTGGAAATATTT | | | CAACAGAAACCAGGACAGCC | | |
| | ATCCTGGTAGTAGTGATACTAA | | | AACCAAACTCCTTATCTATA | | |
| | CTACAATGAGAAGTTCAAGCGC | | | CTGCATCCAACCTAGAATCT | | |
| | AAGGCCACACTGACTGTAGACA | | | GGAGTTCCTGACAGGTTCAG | | |
| | CATCCTCCAGCACAGCCTACAT | | | TGGCAGTGGGTCTGGGACAG | | |
| | GCAGCTCAGCAGCCTGACATCT | | | ACTTCACCCTCAACATCCAT | | |
| | GACGACTCTGCGGTCTATTATT | | | CCTGTGGAGGAGGAGGATGC | | |
| | GTGCAAGACGGCCTCTGTTCCA | | | TGCAACCTATTACTGTCAGC | | |
| | TGCTATGGACTACTGGGGTCAA | | | AAAGCAAGGAGTTTCCGTGG | | |
| | GGAACCTCAGTCACCGTCTCCT | | | ACGTTCGGTGGAGGCACCAA | | |
| | CA | | | GTTGGAAATCAAA | | |

In some embodiments, the antibody or an antigen binding fragment thereof comprises, or consists essentially of, or yet further consists of one or more of:
(SCT-Pa01) a heavy chain variable domain that comprises, or consists essentially of, or yet further consists of SEQ ID NO: 4 or an equivalent thereof comprising, or consisting essentially of, or yet further consisting of the CDRs of the variable domain;
(SCT-Pa02) a heavy chain variable domain that comprises, or consists essentially of, or yet further consists of SEQ ID NO: 5 or an equivalent thereof comprising, or consisting essentially of, or yet further consisting of the CDRs of the variable domain;
(SCT-Pa03) a heavy chain variable domain that comprises, or consists essentially of, or yet further consists of SEQ ID NO: 6 or an equivalent thereof comprising, or consisting essentially of, or yet further consisting of the CDRs of the variable domain;
(SCT-Pa04) a heavy chain variable domain that comprises, or consists essentially of, or yet further consists of SEQ ID NO: 7 or an equivalent thereof comprising, or consisting essentially of, or yet further consisting of the CDRs of the variable domain;
(SCT-Pa05) a heavy chain variable domain that comprises, or consists essentially of, or yet further consists of SEQ ID NO: 8 or an equivalent thereof comprising, or consisting essentially of, or yet further consisting of the CDRs of the variable domain;
(SCT-Pa06) a heavy chain variable domain that comprises, or consists essentially of, or yet further consists of SEQ ID NO: 9 or an equivalent thereof comprising, or consisting essentially of, or yet further consisting of the CDRs of the variable domain;
(SCT-Pa07) a heavy chain variable domain that comprises, or consists essentially of, or yet further consists of SEQ ID NO: 10 or an equivalent thereof comprising, or consisting essentially of, or yet further consisting of the CDRs of the variable domain;
(SCT-Pa08) a heavy chain variable domain that comprises, or consists essentially of, or yet further consists of SEQ ID NO: 11 or an equivalent thereof comprising, or consisting essentially of, or yet further consisting of the CDRs of the variable domain;
(SCT-Pa09) a heavy chain variable domain that comprises, or consists essentially of, or yet further consists of SEQ ID NO: 12 or an equivalent thereof comprising, or consisting essentially of, or yet further consisting of the CDRs of the variable domain;
(SCT-Pa10) a heavy chain variable domain that comprises, or consists essentially of, or yet further consists of SEQ ID NO: 13 or an equivalent thereof comprising, or consisting essentially of, or yet further consisting of the CDRs of the variable domain;
(SCT-Pa11) a heavy chain variable domain that comprises, or consists essentially of, or yet further consists of SEQ ID NO: 14 or an equivalent thereof comprising, or consisting essentially of, or yet further consisting of the CDRs of the variable domain;
(SCT-Pa12) a heavy chain variable domain that comprises, or consists essentially of, or yet further consists of SEQ ID NO: 15 or an equivalent thereof comprising, or consisting essentially of, or yet further consisting of the CDRs of the variable domain;
(SCT-Pa13) a heavy chain variable domain that comprises, or consists essentially of, or yet further consists of SEQ ID NO: 16 or an equivalent thereof comprising, or consisting essentially of, or yet further consisting of the CDRs of the variable domain;
(SCT-Pa14) a heavy chain variable domain that comprises, or consists essentially of, or yet further consists of SEQ ID NO: 17 or an equivalent thereof comprising, or consisting essentially of, or yet further consisting of the CDRs of the variable domain;
(SCT-Pa15) a heavy chain variable domain that comprises, or consists essentially of, or yet further consists of SEQ ID NO: 18 or an equivalent thereof comprising, or consisting essentially of, or yet further consisting of the CDRs of the variable domain;
(SCT-Pa16) a heavy chain variable domain that comprises, or consists essentially of, or yet further consists of SEQ ID NO: 19 or an equivalent thereof comprising, or consisting essentially of, or yet further consisting of the CDRs of the variable domain;
(SCT-Pa17) a heavy chain variable domain that comprises, or consists essentially of, or yet further consists of SEQ ID NO: 20 or an equivalent thereof comprising, or consisting essentially of, or yet further consisting of the CDRs of the variable domain;
(SCT-Pa18) a heavy chain variable domain that comprises, or consists essentially of, or yet further consists of SEQ ID NO: 21 or an equivalent thereof comprising, or consisting essentially of, or yet further consisting of the CDRs of the variable domain;
(SCT-Pa19) a heavy chain variable domain that comprises, or consists essentially of, or yet further consists of SEQ ID NO: 22 or an equivalent thereof comprising, or consisting essentially of, or yet further consisting of the CDRs of the variable domain; or
(SCT-Pa20) a heavy chain variable domain that comprises, or consists essentially of, or yet further consists of SEQ ID NO: 23 or an equivalent thereof comprising, or consisting essentially of, or yet further consisting of the CDRs of the variable domain.

Additionally or alternatively, the antibody or antigen binding fragment comprises, or consists essentially of, or yet further consists of one or more of:
(SCT-Pa01) a light chain variable domain that comprises, or consists essentially of, or yet further consists of SEQ ID NO: 24 or an equivalent thereof comprising, or consisting essentially of, or yet further consisting of the CDRs of the variable domain;
(SCT-Pa02) a light chain variable domain that comprises, or consists essentially of, or yet further consists of SEQ ID NO: 25 or an equivalent thereof comprising, or consisting essentially of, or yet further consisting of the CDRs of the variable domain;
(SCT-Pa03) a light chain variable domain that comprises, or consists essentially of, or yet further consists of SEQ ID NO: 26 or an equivalent thereof comprising, or consisting essentially of, or yet further consisting of the CDRs of the variable domain;
(SCT-Pa04) a light chain variable domain that comprises, or consists essentially of, or yet further consists of SEQ ID NO: 27 or an equivalent thereof comprising, or consisting essentially of, or yet further consisting of the CDRs of the variable domain;
(SCT-Pa05) a light chain variable domain that comprises, or consists essentially of, or yet further consists of SEQ ID NO: 28 or an equivalent thereof comprising, or consisting essentially of, or yet further consisting of the CDRs of the variable domain;
(SCT-Pa06) a light chain variable domain that comprises, or consists essentially of, or yet further consists of SEQ ID NO: 29 or an equivalent thereof comprising, or consisting essentially of, or yet further consisting of the CDRs of the variable domain;
(SCT-Pa07) a light chain variable domain that comprises, or consists essentially of, or yet further consists of SEQ ID NO: 30 or an equivalent thereof comprising, or consisting essentially of, or yet further consisting of the CDRs of the variable domain;
(SCT-Pa08) a light chain variable domain that comprises, or consists essentially of, or yet further consists of SEQ ID NO: 31 or an equivalent thereof comprising, or consisting essentially of, or yet further consisting of the CDRs of the variable domain;
(SCT-Pa09) a light chain variable domain that comprises, or consists essentially of, or yet further consists of SEQ ID NO: 32 or an equivalent thereof comprising, or consisting essentially of, or yet further consisting of the CDRs of the variable domain;
(SCT-Pa10) a light chain variable domain that comprises, or consists essentially of, or yet further consists of SEQ ID NO: 33 or an equivalent thereof comprising, or consisting essentially of, or yet further consisting of the CDRs of the variable domain;
(SCT-Pa11) a light chain variable domain that comprises, or consists essentially of, or yet further consists of SEQ ID NO: 34 or an equivalent thereof comprising, or consisting essentially of, or yet further consisting of the CDRs of the variable domain;
(SCT-Pa12) a light chain variable domain that comprises, or consists essentially of, or yet further consists of SEQ ID NO: 35 or an equivalent thereof comprising, or consisting essentially of, or yet further consisting of the CDRs of the variable domain;
(SCT-Pa13) a light chain variable domain that comprises, or consists essentially of, or yet further consists of SEQ ID NO: 36 or an equivalent thereof comprising, or consisting essentially of, or yet further consisting of the CDRs of the variable domain;
(SCT-Pa14) a light chain variable domain that comprises, or consists essentially of, or yet further consists of SEQ ID NO: 37 or an equivalent thereof comprising, or consisting essentially of, or yet further consisting of the CDRs of the variable domain;
(SCT-Pa15) a light chain variable domain that comprises, or consists essentially of, or yet further consists of SEQ ID NO: 38 or an equivalent thereof comprising, or consisting essentially of, or yet further consisting of the CDRs of the variable domain;
(SCT-Pa16) a light chain variable domain that comprises, or consists essentially of, or yet further consists of SEQ ID NO: 39 or an equivalent thereof comprising, or consisting essentially of, or yet further consisting of the CDRs of the variable domain;
(SCT-Pa17) a light chain variable domain that comprises, or consists essentially of, or yet further consists of SEQ ID NO: 40 or an equivalent thereof comprising, or consisting essentially of, or yet further consisting of the CDRs of the variable domain;
(SCT-Pa18) a light chain variable domain that comprises, or consists essentially of, or yet further consists of SEQ ID NO: 41 or an equivalent thereof comprising, or consisting essentially of, or yet further consisting of the CDRs of the variable domain;
(SCT-Pa19) a light chain variable domain that comprises, or consists essentially of, or yet further consists of SEQ ID NO: 42 or an equivalent thereof comprising, or consisting essentially of, or yet further consisting of the CDRs of the variable domain; or
(SCT-Pa20) a light chain variable domain that comprises, or consists essentially of, or yet further consists of SEQ ID NO: 43 or an equivalent thereof comprising, or consisting essentially of, or yet further consisting of the CDRs of the variable domain.

In some embodiments, the antibody or an antigen binding fragment thereof comprises, or consists essentially of, or yet further consists of one or more of:
(SCT-Pa01) a heavy chain variable domain that comprises, or consists essentially of, or yet further consists of SEQ ID NO: 4 and a light chain variable domain that comprises, or consists essentially of, or yet further consists of SEQ ID NO: 24, or an equivalent of each thereof comprising, or consisting essentially of, or yet further consisting of CDRs of the variable domain;
(SCT-Pa02) a heavy chain variable domain that comprises, or consists essentially of, or yet further consists of SEQ ID NO: 5 and a light chain variable domain that comprises, or consists essentially of, or yet further consists of SEQ ID NO: 25, or an equivalent of each thereof comprising, or consisting essentially of, or yet further consisting of CDRs of the variable domain;
(SCT-Pa03) a heavy chain variable domain that comprises, or consists essentially of, or yet further consists of SEQ ID NO: 6 and a light chain variable domain that comprises, or consists essentially of, or yet further consists of SEQ ID NO: 26, or an equivalent of each thereof comprising, or consisting essentially of, or yet further consisting of CDRs of the variable domain;
(SCT-Pa04) a heavy chain variable domain that comprises, or consists essentially of, or yet further consists of SEQ ID NO: 7 and a light chain variable domain that comprises, or consists essentially of, or yet further consists of SEQ ID NO: 27, or an equivalent of each thereof comprising, or consisting essentially of, or yet further consisting of CDRs of the variable domain;
(SCT-Pa05) a heavy chain variable domain that comprises, or consists essentially of, or yet further consists of SEQ ID NO: 8 and a light chain variable domain that comprises, or consists essentially of, or yet further consists of SEQ ID NO: 28, or an equivalent of each thereof comprising, or consisting essentially of, or yet further consisting of CDRs of the variable domain;
(SCT-Pa06) a heavy chain variable domain that comprises, or consists essentially of, or yet further consists of SEQ ID NO: 9 and a light chain variable domain that comprises, or consists essentially of, or yet further consists of SEQ ID NO: 29, or an equivalent of each thereof comprising, or consisting essentially of, or yet further consisting of CDRs of the variable domain;
(SCT-Pa07) a heavy chain variable domain that comprises, or consists essentially of, or yet further consists of SEQ ID NO: 10 and a light chain variable domain that comprises, or consists essentially of, or yet further consists of SEQ ID NO: 30, or an equivalent of each thereof comprising, or consisting essentially of, or yet further consisting of CDRs of the variable domain;
(SCT-Pa08) a heavy chain variable domain that comprises, or consists essentially of, or yet further consists of SEQ ID NO: 11 and a light chain variable domain that comprises, or consists essentially of, or yet further consists of SEQ ID NO: 31, or an equivalent of each thereof comprising, or consisting essentially of, or yet further consisting of CDRs of the variable domain;
(SCT-Pa09) a heavy chain variable domain that comprises, or consists essentially of, or yet further consists of SEQ ID NO: 12 and a light chain variable domain that comprises, or consists essentially of, or yet further consists of SEQ ID NO: 32, or an equivalent of each thereof comprising, or consisting essentially of, or yet further consisting of CDRs of the variable domain;
(SCT-Pa10) a heavy chain variable domain that comprises, or consists essentially of, or yet further consists of SEQ ID NO: 13 and a light chain variable domain that comprises, or consists essentially of, or yet further consists of SEQ ID NO: 33, or an equivalent of each thereof comprising, or consisting essentially of, or yet further consisting of CDRs of the variable domain;
(SCT-Pa11) a heavy chain variable domain that comprises, or consists essentially of, or yet further consists of SEQ ID NO: 14 and a light chain variable domain that comprises, or consists essentially of, or yet further consists of SEQ ID NO: 34, or an equivalent of each thereof comprising, or consisting essentially of, or yet further consisting of CDRs of the variable domain;
(SCT-Pa12) a heavy chain variable domain that comprises, or consists essentially of, or yet further consists of SEQ ID NO: 15 and a light chain variable domain that comprises, or consists essentially of, or yet further consists of SEQ ID NO: 35, or an equivalent of each thereof comprising, or consisting essentially of, or yet further consisting of CDRs of the variable domain;
(SCT-Pa13) a heavy chain variable domain that comprises, or consists essentially of, or yet further consists of SEQ ID NO: 16 and a light chain variable domain that comprises, or consists essentially of, or yet further consists of SEQ ID NO: 36, or an equivalent of each thereof comprising, or consisting essentially of, or yet further consisting of CDRs of the variable domain;
(SCT-Pa14) a heavy chain variable domain that comprises, or consists essentially of, or yet further consists of SEQ ID NO: 17 and a light chain variable domain that comprises, or consists essentially of, or yet further consists of SEQ ID NO: 37, or an equivalent of each thereof comprising, or consisting essentially of, or yet further consisting of CDRs of the variable domain;
(SCT-Pa15) a heavy chain variable domain that comprises, or consists essentially of, or yet further consists of SEQ ID NO: 18 and a light chain variable domain that comprises, or consists essentially of, or yet further consists of SEQ ID NO: 38, or an equivalent of each thereof comprising, or consisting essentially of, or yet further consisting of CDRs of the variable domain;
(SCT-Pa16) a heavy chain variable domain that comprises, or consists essentially of, or yet further consists of SEQ ID NO: 19 and a light chain variable domain that comprises, or consists essentially of, or yet further consists of SEQ ID NO: 39, or an equivalent of each thereof comprising, or consisting essentially of, or yet further consisting of CDRs of the variable domain;
(SCT-Pa17) a heavy chain variable domain that comprises, or consists essentially of, or yet further consists of SEQ ID NO: 20 and a light chain variable domain that comprises, or consists essentially of, or yet further consists of SEQ ID NO: 40, or an equivalent of each thereof comprising, or consisting essentially of, or yet further consisting of CDRs of the variable domain;
(SCT-Pa18) a heavy chain variable domain that comprises, or consists essentially of, or yet further consists of SEQ ID NO: 21 and a light chain variable domain that comprises, or consists essentially of, or yet further consists of SEQ ID NO: 41, or an equivalent of each thereof comprising, or consisting essentially of, or yet further consisting of CDRs of the variable domain;
(SCT-Pa19) a heavy chain variable domain that comprises, or consists essentially of, or yet further consists of SEQ ID NO: 22 and a light chain variable domain that comprises, or consists essentially of, or yet further consists of SEQ ID NO: 42, or an equivalent of each thereof comprising, or consisting essentially of, or yet further consisting of CDRs of the variable domain; or
(SCT-Pa20) a heavy chain variable domain that comprises, or consists essentially of, or yet further consists of SEQ ID NO: 23 and a light chain variable domain that comprises, or consists essentially of, or yet further consists of SEQ ID NO: 43, or an equivalent of each thereof comprising, or consisting essentially of, or yet further consisting of CDRs of the variable domain.

In some embodiments, the equivalent is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, or more identical to the reference sequence that in some cases, is determined using the Clustal Omega alignment program.

In some embodiments, the antibody or antigen binding fragment of specifically recognizes and binds to a nucleocapsid (N) protein of severe acute respiratory syndrome coronavirus 2 (SARS-CoV-2) or an immunogenic fragment thereof. In some embodiments, the N protein comprises, or consists essentially of, or yet further consists of the amino acid sequence of SEQ ID NO: 1 or a natural variant thereof.

In some embodiments, the antibody or antigen binding fragment is isolated or recombinant.

In some embodiments, the antibody or antigen binding fragment is monospecific. In other embodiments, the antibody or antigen binding fragment is multispecific, such as bispecific, e.g., binding to two or more epitopes. In further embodiments, the two or more epitopes are all epitopes of an N protein or a fragment thereof. In other embodiments, at least one of the two or more epitopes is an epitope of an N protein or a fragment thereof. In further embodiments, at least one of the two or more epitopes are of a protein other than an N protein, such as IL-6, IL-6 receptor, IL-1β, IL-17A, or VCAM-1. Additionally or alternatively, a multispecific antibody or antigen binding fragment thereof binding to an epitope of a protein other than an N protein inhibits undesirable inflammation response, such as cytokine storm, in a subject. In further embodiments, the multispecific antibody is an inhibitor of an inflammatory cytokine, such as IL-6 etc.

In some embodiments, the antibody, or antigen binding fragment thereof, is a chimeric, humanized, or single chain antibody, or a humanized single chain antibody.

In some embodiments, the antigen binding fragment is a Fab, F(ab′)₂, Fab′, scFv, or Fv.

In some embodiments, provided is an antibody or antigen binding fragment thereof that competes with any one of an antibody or antigen binding fragment as disclosed herein for binding to SARS-CoV-2, an N protein thereof, or a fragment of the N protein.

In some embodiments, the antibody or antigen binding fragment further comprises a light chain constant domain. In further embodiments, the constant domain is a human constant domain. In some embodiments, the constant domain comprises, or consists essentially of, or yet further consists of a constant domain of a human κ light chain. In some embodiments, the constant domain of the human κ light chain comprises, or consists essentially of, or yet further consists of RTVAAPSVFIFPPSDEQLKSGTASVVCLLNNFYPREAKVQWKVDNALQSGNSQESVT EQDSKDSTYSLSSTLTLSKADYEKHKVYACEVTHQGLSSPVTKSFNRGEC (SEQ ID NO:191). In some embodiments, the constant domain comprises, or consists essentially of, or yet further consists of a constant domain of a human λ light chain. In some embodiments, the constant domain comprises, or consists essentially of, or yet further consists of a constant domain of a λ1 or λ2 or λ3 or λ4 light chain.

In some embodiments, the antibody or antigen binding fragment further comprises a fragment crystallizable region (Fc region). In further embodiments, the Fc region is a human Fc region. In some embodiments, the Fc region comprises, or consists essentially of, or yet further consists of one or more of: an IgG Fc region, an IgA Fc region, an IgD Fc region, an IgM Fc region, or an IgE Fc region. In some embodiments, the Fc region comprises, or consists essentially of, or yet further consists of one or more of: an IgG1 Fc region, an IgG2 Fc region, an IgG3 Fc region, or an IgG4 Fc region. In some embodiments, the human IgG1 Fc region comprises, or consists essentially of, or yet further consists of ASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAP ELLGGPSVFLFPPKPKDTLMISRTPEVTCVVVDVSHEDPEVKFNWYVDGVEVHNAK TKPREEQYNSTYRVVSVLTVLHQDWLNGKEYKCKVSNKALPAPIEKTISKAKGQPR EPQVYTLPPSRDELTKNQVSLTCLVKGFYPSDIAVEWESNGQPENNYKTTPPVLDSD GSFFLYSKLTVDKSRWQQGNVFSCSVMHEALHNHYTQKSLSLSPGK (SEQ ID NO:190).

In some embodiments, the antibody or antigen binding fragment is post-translationally modified, such as glycosylated, hydroxylated, methylated, lapidated, acetylated, SUMOylated, phosphorylated, PEGylated, or any combination thereof.

In some embodiments, the antibody or antigen binding fragment further comprises a detectable or purification marker.

Table 1 provides a summary of the SARS-CoV-2 N protein-specific antibodies described herein.

### Polypeptide

In one aspect, provided is a polypeptide comprising, or consisting essentially of, or yet further consisting of an antibody or an antigen binding fragment thereof as disclosed herein. Additionally or alternatively, provided is a polypeptide comprising, or consisting essentially of, or yet further consisting of a variable domain of the antibody or an antigen binding fragment thereof as disclosed herein. In further embodiments, the polypeptide comprises, or alternatively consists essentially of, or yet further consists of a heavy chain variable domain as disclosed herein. Additionally or alternatively, the polypeptide comprises, or alternatively consists essentially of, or yet further consists of a light chain variable domain as disclosed herein. In yet further embodiments, the polypeptide comprises, or alternatively consists essentially of, or yet further consists of a heavy chain variable domain as disclosed herein and a light chain variable domain as disclosed herein. In some embodiments, the polypeptide comprises, or alternatively consists essentially of, or yet further consists of a heavy chain variable domain as disclosed herein and a light chain variable domain as identified in the same row of Table 3.

### Polynucleotides and Vectors

In one aspect, provided is a polynucleotide encoding an antibody or antigen binding fragment or a polypeptide as disclosed herein, or a polynucleotide complementary thereto.

In a further aspect, provided is a vector comprising, or consisting essentially of, or yet further consisting of a polynucleotide as disclosed herein.

In some embodiments, the vector further comprises a regulatory sequence that directs the expression of the antibody or antigen binding fragment. In some embodiments, the vector further comprises a regulatory sequence that directs the expression of the polynucleotide. In further embodiments, the regulatory sequence comprises, or consists essentially of, or yet further consists of one or more of: a promoter, an enhancer, or a polyadenylation sequence. Accordingly, such vector can be used for producing the antibody or antigen binding fragment or polypeptide as disclosed herein. Such vector can also be used in a gene therapy delivering the antibody or antigen binding fragment or polypeptide as disclosed herein to a subject in need thereof.

In some embodiments, the vector further comprises a regulatory sequence that directs the replication of the polynucleotide. Accordingly, such vector can be used for producing the polynucleotide as disclosed herein.

In some embodiments, the vector is a non-viral vector, such as a plasmid, a nanoparticle or a liposome. In some embodiments, the vector is a viral vector, such as an adenoviral vector, an adeno-associated viral vector, a retroviral vector, a lentiviral vector, or a plant viral vector.

### Cells

In one aspect, provided is a cell comprising one or more of: an antibody or antigen binding fragment as disclosed herein, a polypeptide as disclosed herein, a polynucleotide as disclosed herein, or a vector as disclosed herein.

In some embodiments, the cell is a prokaryotic cell, such as an Escherichia coli cell. In some embodiments, the cell is a eukaryotic cell, such as a mammal cell, an insect cell, or a yeast cell. In further embodiments, the mammal cell is an HEK293 cell. In some embodiment, the cell is a Chinese hamster ovary cell.

In some embodiments, the cells as disclosed herein are suitable for use in a cell therapy and delivering an antibody or antigen binding fragment thereof or polypeptide as disclosed herein to a subject in need thereof.

In some embodiments, the cells as disclosed herein are suitable for producing an antibody or antigen binding fragment thereof or polypeptide as disclosed herein.

In a further aspect, provided is a hybridoma expressing an antibody or antigen binding fragment as disclosed herein. In some embodiments, the hybridoma comprises one or more of: an antibody or antigen binding fragment as disclosed herein, a polynucleotide as disclosed herein, or a vector as disclosed herein.

### Methods of Production

In one aspect, provided is a method of producing an antibody or an antigen binding fragment as disclosed herein.

In some embodiments, the method comprises, or consists essentially of, or yet further consists of culturing a cell comprising a polynucleotide encoding the antibody or the antigen binding fragment under conditions suitable for expression of the antibody or antigen binding fragment. In some embodiments, the method further comprises introducing the polynucleotide to the cell prior to the culturing step.

In some embodiments, the method comprises, or consists essentially of, or yet further consists of culturing a hybridoma as disclosed herein under conditions suitable for expression of the antibody or antigen binding fragment.

In some embodiments, the method comprises, or consists essentially of, or yet further consists of contacting a polynucleotide as disclosed herein or a vector as disclosed herein with an RNA polymerase, adenosine triphosphate (ATP), cytidine triphosphate (CTP), guanosine-5′-triphosphate (GTP), and uridine triphosphate (UTP) under conditions suitable for transcription to messenger RNA, and contacting the transcribed messenger RNA with a ribosome, tRNAs, an aminoacyl-tRNA synthetase, and initiation, elongation and termination factors under conditions suitable for translation to the antibody or antigen binding fragment. In some embodiments, the method comprises, or consists essentially of, or yet further consists of contacting the transcribed messenger RNA with a cell lysate comprising, or consisting essentially of, or yet further consisting of the ribosome, tRNAs, aminoacyl-tRNA synthetase, and initiation, elongation and termination factors under conditions.

In some embodiments, a method as disclosed herein further comprises isolating the expressed antibody or antigen binding fragment.

### Compositions

Also provided is a composition comprising, or consisting essentially of, or yet further consisting of a carrier (such as a pharmaceutically acceptable carrier) and one or more of: an antibody or antigen binding fragment as disclosed herein, a polypeptide as disclosed herein, a polynucleotide as disclosed herein, a vector as disclosed herein, a cell as disclosed herein, or a hybridoma as disclosed herein.

In some embodiments, the composition comprises, or consists essentially of, or yet further consists of two or more of the antibodies or antigen binding fragments as disclosed herein. In further embodiments, the two or more of the antibodies or antigen binding fragments recognize and bind to at least two different epitopes.

In one aspect, the present disclosure provides a composition or kit comprising, or consisting essentially of, or yet further consisting of an antibody or antigen-binding fragment as described herein in association with a further therapeutic agent (also referred to herein as a combination therapy).

In one aspect, the present disclosure provides a pharmaceutical composition comprising, or consisting essentially of, or yet further consisting of an antibody or antigen binding fragment thereof as disclosed herein, and a pharmaceutically acceptable carrier, such as a diluent. In some embodiments, the pharmaceutical composition comprises one or more excipients.

In one aspect, the present disclosure provides a pharmaceutical composition comprising, or consisting essentially of, or yet further consisting of an antigen-binding protein, antibody or antigen-binding fragment as disclosed herein and a pharmaceutically acceptable carrier and, optionally, a further therapeutic agent.

In some embodiments, the further therapeutic agent comprises, or consists essentially of, or yet further consists of an anti-viral drug or a vaccine or both. In some embodiments, the further therapeutic agent comprises, or consists essentially of, or yet further consists of one or more of: an anti-inflammatory agent, or an antimalarial agent, or both. In some cases, the antimalarial agent comprises, or consists essentially of, or yet further consists of chloroquine or hydroxychloroquine or both. In some cases, the anti-inflammatory agent comprises, or consists essentially of, or yet further consists of an antibody, such as sarilumab, tocilizumab, or gimsilumab. In some embodiments, the further therapeutic agent is a second antibody or antigen-binding fragment as disclosed herein.

### Methods of Uses and Systems

In certain aspects, provided antibodies disclosed herein can be used for identifying SARS-CoV-2 infected patients by specifically detecting the virus via its N protein. Such a test is absolutely necessary to help protect frontline healthcare workers to isolate those who are infected by the virus and treat them before their illness worsen. Epidemiologists can reliably identify infected subjects in hot spots to better measure the extent of the outbreaks, and government officials can use those results to help decide when and how to return residents to daily life. Most importantly, it is key to economic recovery because the infected population can be identified and quarantined to allow the rest of the society to function and operate.

In one aspect, provided is a method of one or more of: (a) treating a subject having or suspect of having a disease, such as a SARS-CoV-2 infection, (b) conferring anti-SARS-CoV-2 passive immunity to a subject in need thereof, (c) conferring or inducing an immune response to SARS-CoV-2 in a subject in need thereof, (d) neutralizing SARS-CoV-2 in a subject in need thereof, or (e) decreasing viral load of SARS-CoV-2 in a subject. In another aspect, the present disclosure provides a method of reducing, retarding, or otherwise inhibiting growth and/or replication of SARS-CoV-2 in a subject in need thereof, such as those diagnosed as having COVID-19. The method comprises, or consists essentially of, or yet further consists of administering to the subject one or more of: an antibody or antigen binding fragment as disclosed herein, a polypeptide as disclosed herein, a polynucleotide as disclosed herein, a vector as disclosed herein, or a cell as disclosed herein.

In some embodiments, the subject in need is diagnosed of having a disease as disclosed herein. In some embodiments, the subject in need is at risk of having a disease as disclosed herein. In some embodiments, the subject is suspect of having a disease as disclosed herein. In some embodiments, the subject has exposed to a SARS-CoV-2.

In some embodiments, administration of a pharmaceutical composition comprising one or more of the antibodies as described herein can be made to a subject in need thereof, such as those exposed to or suspect of exposed to SARS-CoV-2. In some embodiments, the pharmaceutical compositions as described herein can be administered alone or in combination with other therapies deemed appropriate by a clinician or practitioner. In some embodiments, the pharmaceutical compositions described herein may reduce the number of days of the subject having COVID-19 symptoms by one or more days, such as reducing the days of having symptoms by 1, 2, 3, 4, 5, 6, 7, 8, 9, 10, 11, 12, 13, 14 days.

In some embodiments, the method further comprises treating the subject with a combination therapy. In further embodiments, the combination therapy comprises, or consists essentially of, or yet further consists of one or more of: an anti-viral agent, such as remdesivir, lopinavir, ritonavir, ivermectin, tamiflu, or favipiravir; an anti-inflammatory agent such as dexamethasone, tocilizumab, kevzara, colcrys, hydroxychloroquine, chloroquine, or a kinase inhibitor; a covalescent plasma from a subject recovered from a SARS-CoV-2 infection; an antibody binding to SARS-CoV-2 other than the antibody or antigen binding fragment as disclosed herein, such as bamlanivimab, etesevimab, casirivimab, or imdevimab; an antibiotic agent, such as azithromycin; or a SARS-CoV-2 vaccine.

In some embodiments, the method further comprises testing the subject for SARS-CoV-2 infection. In some embodiments, the testing step comprises, or consists essentially of, or yet further consist of testing a biological sample isolated from a subject, such as nasopharyngeal and oropharyngeal swabs, by real-time reverse-transcriptase-polymerase-chain-reaction (rRT-PCR) assay. A description of this assay and sequence information for the rRT-PCR panel primers and probes are available on the CDC Laboratory Information website for 2019-nCoV (www.cdc.gov/coronavirus/2019-nCoV/lab/index.html), which is incorporated herein by reference in its entirety. In some embodiments, the subject is selected for the administration if the biological sample is tested positive for SARS-CoV-2 infection.

In some embodiments, the subject is selected for the administration if the antibody or antigen binding fragment binds to a component of a biological sample isolated from the subject. In addition or alternatively, other assays including commercially available tests, can be utilized to test the subject for infection.

In some embodiments, the disease, such as COVID-19, is of moderate severity. In some embodiments, administration as described herein is initiated within the earlier of 24 to 72 hours of symptom onset or confirmation of the subject having COVID-19 or exposure to SARS-CoV-2. In some embodiments, administration is initiated within the earlier of 24 hours of symptom onset or confirmation of the subject having COVID-19 or exposure to SARS-CoV-2.

In some embodiments, the subject is at an elevated risk of exposure to SARS-CoV-2. In some embodiments, the subject is a health care worker. In some embodiments, the subject is located in an area where ongoing community spread of SARS-CoV-2 has been reported. In some embodiments, the subject has been in close contacts with one or more persons with COVID-19.

In some embodiments, the subject is at an elevated risk of severe illness. In some embodiments, the subject is 60 years of age or older. In some embodiments, the subject has a serious chronic medical condition. In some embodiments, the chronic medical condition comprises, or consists essentially of, or yet further consists of one or more of: pulmonary disease, diabetes mellitus (type 2), requiring oral medication or insulin for treatment, hypertension, cardiovascular disease.

In some embodiments, the subject has a baseline blood pressure under 110 mmHg systolic at rest. In some embodiments, the subject has a body mass index ≥30.

Also provided is a detection system comprising, or consisting essentially of, or yet further consisting of an antibody or an antigen binding fragment as disclosed herein. In one aspect, the system also contains a detectable marker that produces a detectable signal upon binding of the antibody or antigen binding fragment thereof with a SARS-CoV-2 N protein or an immunogenic fragment thereof. In some embodiments, the system is an enzyme-linked immunosorbent assay (ELISA) or a lateral flow immunoassay.

Accordingly, provided is a method comprising, or consisting essentially of, or yet further consisting of contacting the antibody or antigen binding fragment of a detection system as disclosed herein with a biological sample isolated from a subject. In some embodiments, binding of the antibody or antigen binding fragment thereof with a component of the biological sample indicates the subject has or had a SARS-CoV-2 infection. The systems can also be used to monitor therapy and SARS-CoV-2 infection.

In yet a further aspect, provided is a method for detecting a SARS-CoV-2, an N protein thereof, or an immunogenic fragment of the N protein. The method comprises, or consists essentially of, or yet further consists of contacting the antibody or antigen binding fragment of the detection system with a sample. In some embodiments, the method further comprises contacting the detectable marker with the antibody or antigen binding fragment. In further embodiments, binding of the antibody or antigen binding fragment with a component of the sample indicates presence of a SARS-CoV-2 N protein or an immunogenic fragment in the sample.

### Kits

Additionally provided is a kit comprising, or consisting essentially of, or yet further consisting of an instruction for use in a method as disclosed herein. In some embodiments, the kit further comprises one or more of: an antibody or antigen binding fragment as disclosed herein, a polypeptide as disclosed herein, a polynucleotide as disclosed herein, a vector as disclosed herein, a cell as disclosed herein, a hybridoma as disclosed herein, a composition as disclosed herein, or a system as disclosed herein. Additionally or alternatively, the kit further comprises one or more of: an RNA polymerase, adenosine triphosphate (ATP), cytidine triphosphate (CTP), guanosine-5′-triphosphate (GTP), uridine triphosphate (UTP), a ribosome, tRNAs, an aminoacyl-tRNA synthetase, or initiation, elongation and termination factors.

### EXAMPLES

The following example is put forth so as to provide those of ordinary skill in the art with a complete description of how to make and use the present disclosure, and is not intended to limit the scope of what the inventors regard as their disclosure nor is it intended to represent that the experiment below is all or the only experiment that could be performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Isolation of Murine Anti-SARS-CoV-2 N Protein Antibodies

Immunization & single cell suspension generation: Recombinant SARS-CoV-2 N protein His tagged catalog no: NUN-C5227 (ACROBiosystems, Beijing, China, SEQ ID NO:2) was used to immunize young CD-1 mice each with 80 μg of the protein in SIGMA ADJUVANT SYSTEM® (Sigma-Aldrich, St. Louis, MO) over a period of 35 days using a rapid immunization protocol of Antibody Solutions (Santa Clara, CA). The lymph nodes were harvested on day 35. Single cell suspension of the lymph node was generated, and the suspension was filtered through a 70 μm mesh (BD Bioscience) to remove clumps.

Plasma cell isolation, antibody capture, and antigen interrogation: The filtered lymphocyte suspension was enriched for plasma cells actively secreting IgGs using a kit based on cell surface expression of CD138 (Miltenyi, Auburn, CA). Using a method described in U.S. Pat. No. 9,328,172, freshly enriched plasma cells were deposited on a PDMS device to allow a single cell settled in the microwells on the device. Antibody secreted from each plasma cell was captured on a derivatized microscope slide. Antigen-specific antibody secreting cells were identified by interrogating the antibody capture slide with varying concentrations of fluorescently labeled SARS-CoV-2 N protein tagged with His (ACROBiosystems, Beijing, China, catalog no: NUN-C5227, SEQ ID NO:2) and counter-screen with fluorescently labeled human tumor necrosis factor receptor 2 protein, His tag (Sino Biological, Beijing, China, catalog no: 10417-H08H, SEQ ID NO: 3) to detect recognizing and/or neutralizing antibodies. Labeling was done using a kit (AnaSpec, Fremont, CA, AS-72046, ANATAG™ HILYTE™ Fluor 555 Microscale Protein Labeling Kit *Ultra Convenient*).

mRNA capture: After antibody capture, the medium was removed, and replaced with lysis buffer followed by prompt closure of the top of the microwells with a custom oligonucleotide microarray (Agilent, Santa Clara, CA). This procedure was previously described in U.S. Pat. No. 9,328,172. The custom oligonucleotide microarray is prepared such that each feature contains not only a unique tag specifying its coordinate but also capture probes for all subclasses (1, 2a, 2b, and 3) of murine IgG heavy chain, murine Ig kappa light chain.

cDNA synthesis, PCR amplification, and next generation sequencing: Captured mRNA on the custom microarray was further processed to synthesize cDNA of each mRNA incorporating the unique tag originally on each feature. The cDNA is then amplify using a Taq polymerase (Promega, Madison, WI) and appropriate set of primers to allow amplification of the following genes: variable domain of IgG heavy chain subclasses and variable domain of Ig kappa light chain. Though now released from cells, these fragments of each gene are now labeled with the unique tag from the custom oligonucleotide microarray manifesting their originating locations. The amplicons were further manipulated to have appropriate sequence attached at both ends to enable sequencing on an Illumina MiSeq instrument using 2×250 bp chemistry at SeqMatic LLC (Fremont, CA).

Bioinformatic analysis of images and DNA sequences: Sequencing reads from MiSeq were processed and the embedded tag in each read was identified and converted into coordinates. The coordinates were plotted to yield a synthetic map of the mRNA recovered. Most of the coordinates form clusters that designate the location of the originating cell for the recovered mRNA sequences. Next, CDR3 motif present in each read with the coordinates was identified and collated according to the clusters that matched the location of an antibody spot visualized by an appropriate fluorescently labeled secondary antibody. Identical or nearly identical CDR3s for a given antibody spot were organized and form consistent pair of V_{H} and V_{L} sequences. The remaining part of V_{H} or V_{L} sequence containing the identified CDR3s was identified and the associated sequencing reads were assembled into full-length cDNA sequences for V_{H} and V_{L}. The pair of full-length cDNA was correlated with the affinity measurements associated with each of the antigen-specific antibody spot.

### Example 2: Molecular Reconstruction and Recombinant Expression of Anti-SARS-CoV-2 N Antibodies

The paired V_{H} and V_{L} anti-SARS-CoV-2 N antibody sequences were used to synthesize corresponding gene fragments by a service provider according to the known art. The resulting V_{H} and V_{L} gene fragments were cloned into an appropriate plasmid vector that each contains mouse IgG1 heavy chain constant region (SEQ ID NO:188) and mouse kappa light chain constant region (SEQ ID NO:189), respectively, and transfected into an appropriate mammalian host, such as HEK293, for recombinant expression to produce an antibody preparation in full-IgG format. The antibody preparations were characterized by measurements at OD280 to assess the amount produced and by gel electrophoresis on PAGE to assess the size of the antibody chains produced.

### Example 3: Characterization of Recombinant Anti-SARS-CoV-2 N Antibodies—Affinity Measurements of Individual Antibodies to the N Protein

The recombinantly expressed antibodies were used to assess binding activity to recombinant SARS-CoV-2 N protein (SEQ ID NO:2) by biolayer interferometry (BLI) on an Octet Red 96E instrument at 30° C. in kinetics buffer (1×PBS, 0.05% BSA, 0.02% Tween20). Biosensors were equilibrated for 10 min in kinetics buffer and microplates filled with sample in kinetic buffer and agitated at 1000 rpm. For kinetic analysis of these antibodies, anti-mouse Fc (AMC) biosensors were loaded with recombinant anti-SARS-CoV-2 N protein mAbs (1-2 μg/mL). The loaded sensors were exposed to recombinant SARS-CoV-2 N protein (15 μg/mL) with subtracted buffer reference and buffer dissociated in two independent assays. Data were analyzed in Octet Data Analysis 9.0. To deduce a direct binding affinity via the kinetic rate constants (1/Kads=kdis/kon, where 1/Kads=apparent equilibrium dissociation constant, kon=association rate constant, and koff=dissociation rate constant) the buffer-subtracted Octet data were fit locally (relative binding) or globally (kinetic analysis) to a simple 1:1 Langmuir model. Typical loading levels were 0.8-1.0±0.15 nm within a row of eight tips, with standard deviation within the instrument noise. Purified recombinant anti-SARS-CoV-2 N protein mAbs (100 nM) was associated for 90 s followed by dissociation in buffer for 180 s in two independent assays (R2>0.92) using a non-specific IgG reference. The measured affinity is listed in Table 5.

TABLE 5

| | Kinetic global fit (1:1 binding) | | | |
|---|---|---|---|---|
| SCT mAb ID | KD (M) | kon (1/Ms) | kdis (1/s) | RMax |
| SCT-Pa01 | 1.81E−10 | 4.42E+05 | 8.01E−05 | 0.4206 |
| SCT-Pa02 | 1.00E−09 | 7.30E+05 | 7.30E−04 | 0.3546 |
| SCT-Pa03 | 2.78E−09 | 2.15E+05 | 5.97E−04 | 0.5517 |
| SCT-Pa04 | 3.28E−11 | 4.98E+05 | 1.64E−05 | 0.4059 |
| SCT-Pa05 | 2.89E−10 | 6.24E+05 | 1.80E−04 | 0.3672 |
| SCT-Pa06 | 5.04E−10 | 3.37E+05 | 1.70E−04 | 0.5194 |
| SCT-Pa07 | 1.88E−10 | 4.58E+05 | 8.63E−05 | 0.4292 |
| SCT-Pa08 | 8.97E−10 | 8.60E+05 | 7.72E−04 | 0.078 |
| SCT-Pa09 | 5.31E−11 | 5.03E+05 | 2.67E−05 | 0.3677 |
| SCT-Pa10 | 3.35E−10 | 7.38E+05 | 2.47E−04 | 0.2666 |
| SCT-Pa11 | 1.11E−10 | 5.17E+05 | 5.75E−05 | 0.4209 |
| SCT-Pa12 | 6.49E−10 | 4.28E+05 | 2.78E−04 | 0.3714 |
| SCT-Pa13 | 1.02E−09 | 9.32E+05 | 9.53E−04 | 0.0576 |
| SCT-Pa14 | 1.28E−10 | 5.66E+05 | 7.23E−05 | 0.307 |
| SCT-Pa15 | 9.94E−10 | 8.68E+05 | 8.62E−04 | 0.2629 |
| SCT-Pa16 | 1.49E−09 | 1.04E+06 | 1.54E−03 | 0.0466 |
| SCT-Pa17 | 9.39E−10 | 4.91E+05 | 4.61E−04 | 0.3402 |
| SCT-Pa18 | 2.28E−10 | 5.57E+05 | 1.27E−04 | 0.341 |
| SCT-Pa19 | 2.83E−11 | 4.44E+05 | 1.26E−05 | 0.4366 |
| SCT-Pa20 | 5.37E−12 | 5.76E+05 | 3.09E−06 | 0.3398 |

Antibodies recovered from the antibody campaign described above are listed herein. The CDR sequences and the V_{H} and V_{L} sequences for the anti-SARS-CoV-2 N antibodies described herein are depicted in Tables 2 and 3, respectively.

### Example 4: Characterization of Recombinant Anti-SARS-CoV-2 N Antibodies—Epitope Binning by BLI

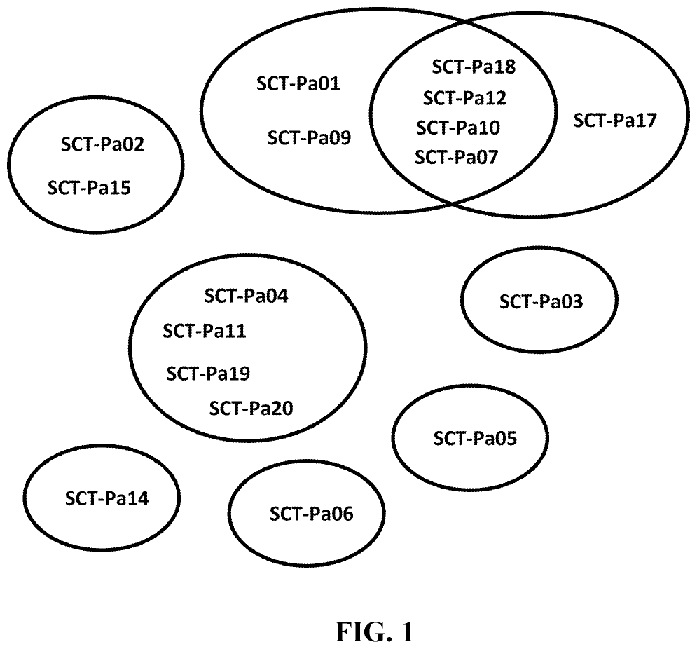

Epitope binning was performed in tandem format using an Octet Red 96 instrument. Recombinant HIS tagged SARS-CoV-2 N protein in kinetics buffer was immobilized onto HIS1K biosensors 10 μg/mL). Each epitope binning experiment consisted of a baseline measurement in kinetics buffer for 60 s; loading of recombinant HIS tagged SARS-CoV-2 N protein for 500 s; a second baseline in kinetics buffer for 60 s; association of the “primary” mAb for 150 s; dissociation in kinetics buffer for 60 s; association of the competing (“secondary”) mAb for 90 s; and a second dissociation in kinetics buffer for 60 s with shaking at 1000 rpm at 30° C. Binding response of the secondary mAb was measured against the saturating mAb. All mAb solutions were prepared in kinetics buffer. All of the data were analyzed using ForteBio Octet Data Analysis software version 10.0. The results of epitope binning are depicted in FIG. 1 for 17 out of the 20 mAbs disclosed herein. Three recombinant mAbs, SCT-Pa08, SCT-Pa13, SCT-Pa16, do not display appreciable binding as when the recombinant SARS-CoV-2 N protein is coated on the biosensor when performing the binning experiments therefore no binning data is available.

### EXEMPLIFIED EMBODIMENTS OF THE DISCLOSURE

Embodiment 1. A recombinant antibody, or antigen binding fragment thereof, that binds to the polypeptide of SEQ ID NO:1, comprising or consisting essentially of or yet further consisting of:
aa. a heavy chain variable domain of SEQ ID NO:4 and a light chain variable domain of SEQ ID NO:24; or
ab. a heavy chain variable domain of SEQ ID NO:5 and a light chain variable domain of SEQ ID NO:25; or
ac. a heavy chain variable domain of SEQ ID NO:6 and a light chain variable domain of SEQ ID NO:26; or
ad. a heavy chain variable domain of SEQ ID NO:7 and a light chain variable domain of SEQ ID NO:27; or
ae. a heavy chain variable domain of SEQ ID NO:8 and a light chain variable domain of SEQ ID NO:28; or
af. a heavy chain variable domain of SEQ ID NO:9 and a light chain variable domain of SEQ ID NO:29; or
ag. a heavy chain variable domain of SEQ ID NO:10 and a light chain variable domain of SEQ ID NO:30; or
ah. a heavy chain variable domain of SEQ ID NO:11 and a light chain variable domain of SEQ ID NO:31; or
ai. a heavy chain variable domain of SEQ ID NO:12 and a light chain variable domain of SEQ ID NO:32; or
aj. a heavy chain variable domain of SEQ ID NO:13 and a light chain variable domain of SEQ ID NO:33; or
ak. a heavy chain variable domain of SEQ ID NO:14 and a light chain variable domain of SEQ ID NO:34; or
al. a heavy chain variable domain of SEQ ID NO:15 and a light chain variable domain of SEQ ID NO:35; or
am. a heavy chain variable domain of SEQ ID NO:16 and a light chain variable domain of SEQ ID NO:36; or
an. a heavy chain variable domain of SEQ ID NO:17 and a light chain variable domain of SEQ ID NO:37; or
ao. a heavy chain variable domain of SEQ ID NO:18 and a light chain variable domain of SEQ ID NO:38; or
ap. a heavy chain variable domain of SEQ ID NO:19 and a light chain variable domain of SEQ ID NO:39; or
aq. a heavy chain variable domain of SEQ ID NO:20 and a light chain variable domain of SEQ ID NO:40; or
ar. a heavy chain variable domain of SEQ ID NO:21 and a light chain variable domain of SEQ ID NO:41; or
as. a heavy chain variable domain of SEQ ID NO:22 and a light chain variable domain of SEQ ID NO:42; or
at. a heavy chain variable domain of SEQ ID NO:23 and a light chain variable domain of SEQ ID NO:43.

Embodiment 2. The recombinant antibody, or antigen binding fragment thereof, of Embodiment 1, wherein the antibody, or antigen binding fragment thereof, is a chimeric, humanized, or single chain antibody.

The preceding merely illustrates the principles of the disclosure. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. An antibody or antigen binding fragment thereof, comprising one or more of:
(SCT-Pa01) a heavy chain (HC) complementarity determining region (CDR) 1 (HCDR1) that comprises the amino acid sequence of SEQ ID NO: 44, an HC CDR 2 (HCDR2) that comprises the amino acid sequence of SEQ ID NO: 45, and an HC CDR 3 (HCDR3) that comprises the amino acid sequence of SEQ ID NO: 46;
(SCT-Pa02) an HCDR1 that comprises the amino acid sequence of SEQ ID NO: 47, an HCDR2 that comprises the amino acid sequence of SEQ ID NO: 48, and an HCDR3 that comprises the amino acid sequence of SEQ ID NO: 49;
(SCT-Pa03) an HCDR1 that comprises the amino acid sequence of SEQ ID NO: 50, an HCDR2 that comprises the amino acid sequence of SEQ ID NO: 51, and an HCDR3 that comprises the amino acid sequence of SEQ ID NO: 52;
(SCT-Pa04) an HCDR1 that comprises the amino acid sequence of SEQ ID NO: 53, an HCDR2 that comprises the amino acid sequence of SEQ ID NO: 54, and an HCDR3 that comprises the amino acid sequence of SEQ ID NO: 55;
(SCT-Pa05) an HCDR1 that comprises the amino acid sequence of SEQ ID NO: 56, an HCDR2 that comprises the amino acid sequence of SEQ ID NO: 57, and an HCDR3 that comprises the amino acid sequence of SEQ ID NO: 58;
(SCT-Pa06) an HCDR1 that comprises the amino acid sequence of SEQ ID NO: 59, an HCDR2 that comprises the amino acid sequence of SEQ ID NO: 60, and an HCDR3 that comprises the amino acid sequence of SEQ ID NO: 61;
(SCT-Pa07) an HCDR1 that comprises the amino acid sequence of SEQ ID NO: 50, an HCDR2 that comprises the amino acid sequence of SEQ ID NO: 62, and an HCDR3 that comprises the amino acid sequence of SEQ ID NO: 63;
(SCT-Pa08) an HCDR1 that comprises the amino acid sequence of SEQ ID NO: 64, an HCDR2 that comprises the amino acid sequence of SEQ ID NO: 65, and an HCDR3 that comprises the amino acid sequence of SEQ ID NO: 66;
(SCT-Pa09) an HCDR1 that comprises the amino acid sequence of SEQ ID NO: 67, an HCDR2 that comprises the amino acid sequence of SEQ ID NO: 68, and an HCDR3 that comprises the amino acid sequence of SEQ ID NO: 69;
(SCT-Pa10) an HCDR1 that comprises the amino acid sequence of SEQ ID NO: 70, an HCDR2 that comprises the amino acid sequence of SEQ ID NO: 71, and an HCDR3 that comprises the amino acid sequence of SEQ ID NO: 72;
(SCT-Pa11) an HCDR1 that comprises the amino acid sequence of SEQ ID NO: 73, an HCDR2 that comprises the amino acid sequence of SEQ ID NO: 54, and an HCDR3 that comprises the amino acid sequence of SEQ ID NO: 74;
(SCT-Pa12) an HCDR1 that comprises the amino acid sequence of SEQ ID NO: 75, an HCDR2 that comprises the amino acid sequence of SEQ ID NO: 76, and an HCDR3 that comprises the amino acid sequence of SEQ ID NO: 77;
(SCT-Pa13) an HCDR1 that comprises the amino acid sequence of SEQ ID NO: 78, an HCDR2 that comprises the amino acid sequence of SEQ ID NO: 79, and an HCDR3 that comprises the amino acid sequence of SEQ ID NO: 80;
(SCT-Pa14) an HCDR1 that comprises the amino acid sequence of SEQ ID NO: 81, an HCDR2 that comprises the amino acid sequence of SEQ ID NO: 82, and an HCDR3 that comprises the amino acid sequence of SEQ ID NO: 83;
(SCT-Pa15) an HCDR1 that comprises the amino acid sequence of SEQ ID NO: 84, an HCDR2 that comprises the amino acid sequence of SEQ ID NO: 79, and an HCDR3 that comprises the amino acid sequence of SEQ ID NO: 85;
(SCT-Pa16) an HCDR1 that comprises the amino acid sequence of SEQ ID NO: 86, an HCDR2 that comprises the amino acid sequence of SEQ ID NO: 87, and an HCDR3 that comprises the amino acid sequence of SEQ ID NO: 88;
(SCT-Pa17) an HCDR1 that comprises the amino acid sequence of SEQ ID NO: 89, an HCDR2 that comprises the amino acid sequence of SEQ ID NO: 90, and an HCDR3 that comprises the amino acid sequence of SEQ ID NO: 91;
(SCT-Pa18) an HCDR1 that comprises the amino acid sequence of SEQ ID NO: 86, an HCDR2 that comprises the amino acid sequence of SEQ ID NO: 92, and an HCDR3 that comprises the amino acid sequence of SEQ ID NO: 93;
(SCT-Pa19) an HCDR1 that comprises the amino acid sequence of SEQ ID NO: 94, an HCDR2 that comprises the amino acid sequence of SEQ ID NO: 95, and an HCDR3 that comprises the amino acid sequence of SEQ ID NO: 96; or
(SCT-Pa20) an HCDR1 that comprises the amino acid sequence of SEQ ID NO: 97, an HCDR2 that comprises the amino acid sequence of SEQ ID NO: 98, and an HCDR3 that comprises the amino acid sequence of SEQ ID NO: 99.

2. The antibody or antigen binding fragment thereof of claim 1, further comprising one or more of:
(SCT-Pa01) a light chain (LC) complementarity determining region (CDR) 1 (LCDR1) that comprises the amino acid sequence of SEQ ID NO: 100, an LC CDR 2 (LCDR2) that comprises the amino acid sequence of SEQ ID NO: 101, and an LC CDR 3 (LCDR3) that comprises the amino acid sequence of SEQ ID NO: 102;
(SCT-Pa02) an LCDR1 that comprises the amino acid sequence of SEQ ID NO: 103, an LCDR2 that comprises the amino acid sequence of SEQ ID NO: 104, and an LCDR3 that comprises the amino acid sequence of SEQ ID NO: 105;
(SCT-Pa03) an LCDR1 that comprises the amino acid sequence of SEQ ID NO: 106, an LCDR2 that comprises the amino acid sequence of SEQ ID NO: 107, and an LCDR3 that comprises the amino acid sequence of SEQ ID NO: 108;
(SCT-Pa04) an LCDR1 that comprises the amino acid sequence of SEQ ID NO: 109, an LCDR2 that comprises the amino acid sequence of SEQ ID NO: 110, and an LCDR3 that comprises the amino acid sequence of SEQ ID NO: 111;
(SCT-Pa05) an LCDR1 that comprises the amino acid sequence of SEQ ID NO: 112, an LCDR2 that comprises the amino acid sequence of SEQ ID NO: 113, and an LCDR3 that comprises the amino acid sequence of SEQ ID NO: 114;
(SCT-Pa06) an LCDR1 that comprises the amino acid sequence of SEQ ID NO: 115, an LCDR2 that comprises the amino acid sequence of SEQ ID NO: 101, and an LCDR3 that comprises the amino acid sequence of SEQ ID NO: 116;
(SCT-Pa07) an LCDR1 that comprises the amino acid sequence of SEQ ID NO: 117, an LCDR2 that comprises the amino acid sequence of SEQ ID NO: 118, and an LCDR3 that comprises the amino acid sequence of SEQ ID NO: 119;
(SCT-Pa08) an LCDR1 that comprises the amino acid sequence of SEQ ID NO: 120, an LCDR2 that comprises the amino acid sequence of SEQ ID NO: 121, and an LCDR3 that comprises the amino acid sequence of SEQ ID NO: 122;
(SCT-Pa09) an LCDR1 that comprises the amino acid sequence of SEQ ID NO: 123, an LCDR2 that comprises the amino acid sequence of SEQ ID NO: 104, and an LCDR3 that comprises the amino acid sequence of SEQ ID NO: 124;
(SCT-Pa10) an LCDR1 that comprises the amino acid sequence of SEQ ID NO: 125, an LCDR2 that comprises the amino acid sequence of SEQ ID NO: 118, and an LCDR3 that comprises the amino acid sequence of SEQ ID NO: 126;
(SCT-Pa11) an LCDR1 that comprises the amino acid sequence of SEQ ID NO: 127, an LCDR2 that comprises the amino acid sequence of SEQ ID NO: 128, and an LCDR3 that comprises the amino acid sequence of SEQ ID NO: 129;
(SCT-Pa12) an LCDR1 that comprises the amino acid sequence of SEQ ID NO: 130, an LCDR2 that comprises the amino acid sequence of SEQ ID NO: 118, and an LCDR3 that comprises the amino acid sequence of SEQ ID NO: 126;
(SCT-Pa13) an LCDR1 that comprises the amino acid sequence of SEQ ID NO: 131, an LCDR2 that comprises the amino acid sequence of SEQ ID NO: 132, and an LCDR3 that comprises the amino acid sequence of SEQ ID NO: 133;
(SCT-Pa14) an LCDR1 that comprises the amino acid sequence of SEQ ID NO: 117, an LCDR2 that comprises the amino acid sequence of SEQ ID NO: 118, and an LCDR3 that comprises the amino acid sequence of SEQ ID NO: 134;
(SCT-Pa15) an LCDR1 that comprises the amino acid sequence of SEQ ID NO: 135, an LCDR2 that comprises the amino acid sequence of SEQ ID NO: 136, and an LCDR3 that comprises the amino acid sequence of SEQ ID NO: 137;
(SCT-Pa16) an LCDR1 that comprises the amino acid sequence of SEQ ID NO: 138, an LCDR2 that comprises the amino acid sequence of SEQ ID NO: 139, and an LCDR3 that comprises the amino acid sequence of SEQ ID NO: 140;
(SCT-Pa17) an LCDR1 that comprises the amino acid sequence of SEQ ID NO: 141, an LCDR2 that comprises the amino acid sequence of SEQ ID NO: 107, and an LCDR3 that comprises the amino acid sequence of SEQ ID NO: 142;
(SCT-Pa18) an LCDR1 that comprises the amino acid sequence of SEQ ID NO: 130, an LCDR2 that comprises the amino acid sequence of SEQ ID NO: 118, and an LCDR3 that comprises the amino acid sequence of SEQ ID NO: 126;
(SCT-Pa19) an LCDR1 that comprises the amino acid sequence of SEQ ID NO: 143, an LCDR2 that comprises the amino acid sequence of SEQ ID NO: 139, and an LCDR3 that comprises the amino acid sequence of SEQ ID NO: 144; or
(SCT-Pa20) an LCDR1 that comprises the amino acid sequence of SEQ ID NO: 145, an LCDR2 that comprises the amino acid sequence of SEQ ID NO: 146, and an LCDR3 that comprises the amino acid sequence of SEQ ID NO: 147.

3. An antibody pair or antigen binding fragment of each thereof,
wherein the first antibody or antigen binding fragment thereof comprises (SCT-Pa09) an LCDR1 that comprises the amino acid sequence of SEO ID NO: 123, an LCDR2 that comprises the amino acid sequence of SEO ID NO: 104, and an LCDR3 that comprises the amino acid sequence of SEO ID NO: 124; and
the second antibody or antigen binding fragment thereof comprises: (SCT-Pa20) an LCDR1 that comprises the amino acid sequence of SEO ID NO: 145, an LCDR2 that comprises the amino acid sequence of SEO ID NO: 146, and an LCDR3 that comprises the amino acid sequence of SEO ID NO: 147: or
wherein the first antibody or antigen binding fragment thereof comprises (SCT-Pa17) an LCDR1 that comprises the amino acid sequence of SEO ID NO: 141, an LCDR2 that comprises the amino acid sequence of SEO ID NO: 107, and an LCDR3 that comprises the amino acid sequence of SEO ID NO: 142; and
the second antibody or antigen binding fragment thereof comprises (SCT-Pa20) an LCDR1 that comprises the amino acid sequence of SEO ID NO: 145, an LCDR2 that comprises the amino acid sequence of SEO ID NO: 146, and an LCDR3 that comprises the amino acid sequence of SEO ID NO: 147.

4. (canceled)

5. The antibody or antigen binding fragment thereof of claim 1, comprising one or more of:
(SCT-Pa01) a heavy chain (HC) complementarity determining region (CDR) 1 (HCDR1) that comprises the amino acid sequence of SEQ ID NO: 44, an HC CDR 2 (HCDR2) that comprises the amino acid sequence of SEQ ID NO: 45, an HC CDR 3 (HCDR3) that comprises the amino acid sequence of SEQ ID NO: 46, a light chain (LC) complementarity determining region (CDR) 1 (LCDR1) that comprises the amino acid sequence of SEQ ID NO: 100, an LC CDR 2 (LCDR2) that comprises the amino acid sequence of SEQ ID NO: 101, and an LC CDR 3 (LCDR3) that comprises the amino acid sequence of SEQ ID NO: 102;
(SCT-Pa02) an HCDR1 that comprises the amino acid sequence of SEQ ID NO: 47, an HCDR2 that comprises the amino acid sequence of SEQ ID NO: 48, an HCDR3 that comprises the amino acid sequence of SEQ ID NO: 49, an LCDR1 that comprises the amino acid sequence of SEQ ID NO: 103, an LCDR2 that comprises the amino acid sequence of SEQ ID NO: 104, and an LCDR3 that comprises the amino acid sequence of SEQ ID NO: 105;
(SCT-Pa03) an HCDR1 that comprises the amino acid sequence of SEQ ID NO: 50, an HCDR2 that comprises the amino acid sequence of SEQ ID NO: 51, an HCDR3 that comprises the amino acid sequence of SEQ ID NO: 52, an LCDR1 that comprises the amino acid sequence of SEQ ID NO: 106, an LCDR2 that comprises the amino acid sequence of SEQ ID NO: 107, and an LCDR3 that comprises the amino acid sequence of SEQ ID NO: 108;
(SCT-Pa04) an HCDR1 that comprises the amino acid sequence of SEQ ID NO: 53, an HCDR2 that comprises the amino acid sequence of SEQ ID NO: 54, an HCDR3 that comprises the amino acid sequence of SEQ ID NO: 55, an LCDR1 that comprises the amino acid sequence of SEQ ID NO: 109, an LCDR2 that comprises the amino acid sequence of SEQ ID NO: 110, and an LCDR3 that comprises the amino acid sequence of SEQ ID NO: 111;
(SCT-Pa05) an HCDR1 that comprises the amino acid sequence of SEQ ID NO: 56, an HCDR2 that comprises the amino acid sequence of SEQ ID NO: 57, an HCDR3 that comprises the amino acid sequence of SEQ ID NO: 58, an LCDR1 that comprises the amino acid sequence of SEQ ID NO: 112, an LCDR2 that comprises the amino acid sequence of SEQ ID NO: 113, and an LCDR3 that comprises the amino acid sequence of SEQ ID NO: 114;
(SCT-Pa06) an HCDR1 that comprises the amino acid sequence of SEQ ID NO: 59, an HCDR2 that comprises the amino acid sequence of SEQ ID NO: 60, an HCDR3 that comprises the amino acid sequence of SEQ ID NO: 61, an LCDR1 that comprises the amino acid sequence of SEQ ID NO: 115, an LCDR2 that comprises the amino acid sequence of SEQ ID NO: 101, and an LCDR3 that comprises the amino acid sequence of SEQ ID NO: 116;
(SCT-Pa07) an HCDR1 that comprises the amino acid sequence of SEQ ID NO: 50, an HCDR2 that comprises the amino acid sequence of SEQ ID NO: 62, an HCDR3 that comprises the amino acid sequence of SEQ ID NO: 63, an LCDR1 that comprises the amino acid sequence of SEQ ID NO: 117, an LCDR2 that comprises the amino acid sequence of SEQ ID NO: 118, and an LCDR3 that comprises the amino acid sequence of SEQ ID NO: 119;
(SCT-Pa08) an HCDR1 that comprises the amino acid sequence of SEQ ID NO: 64, an HCDR2 that comprises the amino acid sequence of SEQ ID NO: 65, an HCDR3 that comprises the amino acid sequence of SEQ ID NO: 66, an LCDR1 that comprises the amino acid sequence of SEQ ID NO: 120, an LCDR2 that comprises the amino acid sequence of SEQ ID NO: 121, and an LCDR3 that comprises the amino acid sequence of SEQ ID NO: 122;
(SCT-Pa09) an HCDR1 that comprises the amino acid sequence of SEQ ID NO: 67, an HCDR2 that comprises the amino acid sequence of SEQ ID NO: 68, an HCDR3 that comprises the amino acid sequence of SEQ ID NO: 69, an LCDR1 that comprises the amino acid sequence of SEQ ID NO: 123, an LCDR2 that comprises the amino acid sequence of SEQ ID NO: 104, and an LCDR3 that comprises the amino acid sequence of SEQ ID NO: 124;
(SCT-Pa10) an HCDR1 that comprises the amino acid sequence of SEQ ID NO: 70, an HCDR2 that comprises the amino acid sequence of SEQ ID NO: 71, an HCDR3 that comprises the amino acid sequence of SEQ ID NO: 72, an LCDR1 that comprises the amino acid sequence of SEQ ID NO: 125, an LCDR2 that comprises the amino acid sequence of SEQ ID NO: 118, and an LCDR3 that comprises the amino acid sequence of SEQ ID NO: 126;
(SCT-Pa11) an HCDR1 that comprises the amino acid sequence of SEQ ID NO: 73, an HCDR2 that comprises the amino acid sequence of SEQ ID NO: 54, an HCDR3 that comprises the amino acid sequence of SEQ ID NO: 74, an LCDR1 that comprises the amino acid sequence of SEQ ID NO: 127, an LCDR2 that comprises the amino acid sequence of SEQ ID NO: 128, and an LCDR3 that comprises the amino acid sequence of SEQ ID NO: 129;
(SCT-Pa12) an HCDR1 that comprises the amino acid sequence of SEQ ID NO: 75, an HCDR2 that comprises the amino acid sequence of SEQ ID NO: 76, an HCDR3 that comprises the amino acid sequence of SEQ ID NO: 77, an LCDR1 that comprises the amino acid sequence of SEQ ID NO: 130, an LCDR2 that comprises the amino acid sequence of SEQ ID NO: 118, and an LCDR3 that comprises the amino acid sequence of SEQ ID NO: 126;
(SCT-Pa13) an HCDR1 that comprises the amino acid sequence of SEQ ID NO: 78, an HCDR2 that comprises the amino acid sequence of SEQ ID NO: 79, an HCDR3 that comprises the amino acid sequence of SEQ ID NO: 80, an LCDR1 that comprises the amino acid sequence of SEQ ID NO: 131, an LCDR2 that comprises the amino acid sequence of SEQ ID NO: 132, and an LCDR3 that comprises the amino acid sequence of SEQ ID NO: 133;
(SCT-Pa14) an HCDR1 that comprises the amino acid sequence of SEQ ID NO: 81, an HCDR2 that comprises the amino acid sequence of SEQ ID NO: 82, an HCDR3 that comprises the amino acid sequence of SEQ ID NO: 83, an LCDR1 that comprises the amino acid sequence of SEQ ID NO: 117, an LCDR2 that comprises the amino acid sequence of SEQ ID NO: 118, and an LCDR3 that comprises the amino acid sequence of SEQ ID NO: 134;
(SCT-Pa15) an HCDR1 that comprises the amino acid sequence of SEQ ID NO: 84, an HCDR2 that comprises the amino acid sequence of SEQ ID NO: 79, an HCDR3 that comprises the amino acid sequence of SEQ ID NO: 85, an LCDR1 that comprises the amino acid sequence of SEQ ID NO: 135, an LCDR2 that comprises the amino acid sequence of SEQ ID NO: 136, and an LCDR3 that comprises the amino acid sequence of SEQ ID NO: 137;
(SCT-Pa16) an HCDR1 that comprises the amino acid sequence of SEQ ID NO: 86, an HCDR2 that comprises the amino acid sequence of SEQ ID NO: 87, an HCDR3 that comprises the amino acid sequence of SEQ ID NO: 88, an LCDR1 that comprises the amino acid sequence of SEQ ID NO: 138, an LCDR2 that comprises the amino acid sequence of SEQ ID NO: 139, and an LCDR3 that comprises the amino acid sequence of SEQ ID NO: 140;
(SCT-Pa17) an HCDR1 that comprises the amino acid sequence of SEQ ID NO: 89, an HCDR2 that comprises the amino acid sequence of SEQ ID NO: 90, an HCDR3 that comprises the amino acid sequence of SEQ ID NO: 91, an LCDR1 that comprises the amino acid sequence of SEQ ID NO: 141, an LCDR2 that comprises the amino acid sequence of SEQ ID NO: 107, and an LCDR3 that comprises the amino acid sequence of SEQ ID NO: 142;
(SCT-Pa18) an HCDR1 that comprises the amino acid sequence of SEQ ID NO: 86, an HCDR2 that comprises the amino acid sequence of SEQ ID NO: 92, an HCDR3 that comprises the amino acid sequence of SEQ ID NO: 93, an LCDR1 that comprises the amino acid sequence of SEQ ID NO: 130, an LCDR2 that comprises the amino acid sequence of SEQ ID NO: 118, and an LCDR3 that comprises the amino acid sequence of SEQ ID NO: 126;
(SCT-Pa19) an HCDR1 that comprises the amino acid sequence of SEQ ID NO: 94, an HCDR2 that comprises the amino acid sequence of SEQ ID NO: 95, an HCDR3 that comprises the amino acid sequence of SEQ ID NO: 96, an LCDR1 that comprises the amino acid sequence of SEQ ID NO: 143, an LCDR2 that comprises the amino acid sequence of SEQ ID NO: 139, and an LCDR3 that comprises the amino acid sequence of SEQ ID NO: 144; or
(SCT-Pa20) an HCDR1 that comprises the amino acid sequence of SEQ ID NO: 97, an HCDR2 that comprises the amino acid sequence of SEQ ID NO: 98, an HCDR3 that comprises the amino acid sequence of SEQ ID NO: 99, an LCDR1 that comprises the amino acid sequence of SEQ ID NO: 145, an LCDR2 that comprises the amino acid sequence of SEQ ID NO: 146, and an LCDR3 that comprises the amino acid sequence of SEQ ID NO: 147.

6.-10. (canceled)

11. The antibody or antigen binding fragment thereof of claim 1, comprising one or more of:
(SCT-Pa01) a heavy chain variable domain that comprises SEQ ID NO: 4 and a light chain variable domain that comprises SEQ ID NO: 24 or an equivalent of each thereof comprising CDRs of the variable domain;
(SCT-Pa02) a heavy chain variable domain that comprises SEQ ID NO: 5 and a light chain variable domain that comprises SEQ ID NO: 25 or an equivalent of each thereof comprising CDRs of the variable domain;
(SCT-Pa03) a heavy chain variable domain that comprises SEQ ID NO: 6 and a light chain variable domain that comprises SEQ ID NO: 26 or an equivalent of each thereof comprising CDRs of the variable domain;
(SCT-Pa04) a heavy chain variable domain that comprises SEQ ID NO: 7 and a light chain variable domain that comprises SEQ ID NO: 27 or an equivalent of each thereof comprising CDRs of the variable domain;
(SCT-Pa05) a heavy chain variable domain that comprises SEQ ID NO: 8 and a light chain variable domain that comprises SEQ ID NO: 28 or an equivalent of each thereof comprising CDRs of the variable domain;
(SCT-Pa06) a heavy chain variable domain that comprises SEQ ID NO: 9 and a light chain variable domain that comprises SEQ ID NO: 29 or an equivalent of each thereof comprising CDRs of the variable domain;
(SCT-Pa07) a heavy chain variable domain that comprises SEQ ID NO: 10 and a light chain variable domain that comprises SEQ ID NO: 30 or an equivalent of each thereof comprising CDRs of the variable domain;
(SCT-Pa08) a heavy chain variable domain that comprises SEQ ID NO: 11 and a light chain variable domain that comprises SEQ ID NO: 31 or an equivalent of each thereof comprising CDRs of the variable domain;
(SCT-Pa09) a heavy chain variable domain that comprises SEQ ID NO: 12 and a light chain variable domain that comprises SEQ ID NO: 32 or an equivalent of each thereof comprising CDRs of the variable domain;
(SCT-Pa10) a heavy chain variable domain that comprises SEQ ID NO: 13 and a light chain variable domain that comprises SEQ ID NO: 33 or an equivalent of each thereof comprising CDRs of the variable domain;
(SCT-Pa11) a heavy chain variable domain that comprises SEQ ID NO: 14 and a light chain variable domain that comprises SEQ ID NO: 34 or an equivalent of each thereof comprising CDRs of the variable domain;
(SCT-Pa12) a heavy chain variable domain that comprises SEQ ID NO: 15 and a light chain variable domain that comprises SEQ ID NO: 35 or an equivalent of each thereof comprising CDRs of the variable domain;
(SCT-Pa13) a heavy chain variable domain that comprises SEQ ID NO: 16 and a light chain variable domain that comprises SEQ ID NO: 36 or an equivalent of each thereof comprising CDRs of the variable domain;
(SCT-Pa14) a heavy chain variable domain that comprises SEQ ID NO: 17 and a light chain variable domain that comprises SEQ ID NO: 37 or an equivalent of each thereof comprising CDRs of the variable domain;
(SCT-Pa15) a heavy chain variable domain that comprises SEQ ID NO: 18 and a light chain variable domain that comprises SEQ ID NO: 38 or an equivalent of each thereof comprising CDRs of the variable domain;
(SCT-Pa16) a heavy chain variable domain that comprises SEQ ID NO: 19 and a light chain variable domain that comprises SEQ ID NO: 39 or an equivalent of each thereof comprising CDRs of the variable domain;
(SCT-Pa17) a heavy chain variable domain that comprises SEQ ID NO: 20 and a light chain variable domain that comprises SEQ ID NO: 40 or an equivalent of each thereof comprising CDRs of the variable domain;
(SCT-Pa18) a heavy chain variable domain that comprises SEQ ID NO: 21 and a light chain variable domain that comprises SEQ ID NO: 41 or an equivalent of each thereof comprising CDRs of the variable domain;
(SCT-Pa19) a heavy chain variable domain that comprises SEQ ID NO: 22 and a light chain variable domain that comprises SEQ ID NO: 42 or an equivalent of each thereof comprising CDRs of the variable domain; or
(SCT-Pa20) a heavy chain variable domain that comprises SEQ ID NO: 23 and a light chain variable domain that comprises SEQ ID NO: 43 or an equivalent of each thereof comprising CDRs of the variable domain.

12. The antibody or antigen binding fragment of claim 11, wherein the equivalent is at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, or more identical to the reference sequence.

13. The antibody or antigen binding fragment of claim 1, specifically recognizing and binding to a nucleocapsid (N) protein of severe acute respiratory syndrome coronavirus 2 (SARS-CoV-2) or an immunogenic fragment thereof.

14.-15. (canceled)

16. The antibody, or antigen binding fragment thereof, of claim 1, wherein the antibody, or antigen binding fragment thereof, is
(a) a chimeric, humanized, or single chain antibody, or a humanized single chain antibody; or
(b) a Fab, F(ab′)2, Fab′, scFv, or Fv.

17. (canceled)

18. The antibody or antigen binding fragment of claim 1, comprising a light chain constant domain.

19. The antibody or antigen binding fragment of claim 18, wherein the constant domain comprises a constant domain of a human κ light chain.

20. (canceled)

21. The antibody or antigen binding fragment of claim 18, wherein the constant domain comprises a constant domain of a human λ light chain.

22. (canceled)

23. The antibody or antigen binding fragment of claim 1, comprises a fragment crystallizable region (Fc region).

24.-26. (canceled)

27. The antibody or antigen binding fragment of claim 1, wherein the antibody or antigen binding fragment is post-translationally modified optionally glycosylated, hydroxylated, methylated, lapidated, acetylated, SUMOylated, phosphorylated, PEGylated, or any combination thereof.

28. (canceled)

29. A polynucleotide encoding the antibody or antigen binding fragment of claim 1, or a polynucleotide complementary thereto.

30. A vector comprising the polynucleotide of claim 29.

31.-35. (canceled)

36. A cell comprising the polynucleotide of claim 29.

37.-38. (canceled)

39. A hybridoma expressing the antibody or antigen binding fragment of claim 1.

40. A method of producing the antibody or antigen binding fragment of claim 1, comprising culturing a cell comprising a polynucleotide encoding the antibody or the antigen binding fragment under conditions suitable for expression of the antibody or antigen binding fragment.

41.-45. (canceled)

46. A composition comprising a carrier and the antibody or antigen binding fragment of claim 1.

47.-49. (canceled)

50. A method of one or more of:
(a) treating a subject having or suspect of having a SARS-CoV-2 infection,
(b) conferring anti-SARS-CoV-2 passive immunity to a subject in need thereof,
(c) conferring or inducing an immune response to SARS-CoV-2 in a subject in need thereof,
(d) neutralizing SARS-CoV-2 in a subject in need thereof, or
(e) decreasing viral load of SARS-CoV-2 in a subject,
the method comprising administering to the subject the antibody or antigen binding fragment of claim 1.

51.-53. (canceled)

54. A detection system comprising the pair of claim 3 and optionally a detectable marker producing a detectable signal upon binding of the antibody or antigen binding fragment thereof with a SARS-CoV-2 N protein or an immunogenic fragment thereof.

55.-56. (canceled)

57. A kit comprising the antibody or antigen binding fragment of claim 1, and instructions for using the kit.

58. (canceled)
